(19) United States
(12) Patent Application Publication
(10) Pub. No.: US 2011/0217713 A1
(43) Pub. Date: Sep. 8, 2011

(54) Biomarkers For The Identification, Monitoring, And Treatment Of Non-Small Cell Lung Cancer (NSCLC)

(75) Inventors: David T. Weaver, Newton, MA (US); William E. Pierceall, Sudbury, MA (US); Brian Ward, Park City, UT (US)

(73) Assignee: On-Q-ity, Waltham, MA (US)

(21) Appl. No.: 13/041,889

(22) Filed: Mar. 7, 2011

Related U.S. Application Data
(60) Provisional application No. 61/310,831, filed on Mar. 5, 2010.

(51) Int. Cl. C12Q 1/68; G01N 33/53

(52) U.S. Cl. 435/6.12; 436/501; 435/7.92

(57) ABSTRACT

This present invention compositions and methods of treating, diagnosing, prognosing cancer and methods of accessing/monitoring the responsiveness of a cancer cell to a therapeutic compound.

### RELATED APPLICATION

This application claims the benefit of U.S. Ser. No. 61/310,831 filed Mar. 5, 2010 the contents of which are incorporated by reference in its entirety.

### FIELD OF THE INVENTION

The present invention relates generally to the identification of biomarkers and methods of using such biomarkers in the screening, prevention, diagnosis, therapy, monitoring, and prognosis o non-small cell lung cancer. The invention relates to the use of biomarkers and biomarker panels for patient stratification to treatments, responsiveness to treatments, for pharmacodynamic monitoring of drug responses and systemic changes. This invention relates to the use of biomarkers of cancer cells in primary tumors, tumor cells in circulation i.e., circulating tumor cells and tumor cells at metastatic sites in the body, in sites where the cancer resides but is dormant, and in lymph nodes.

### BACKGROUND OF THE INVENTION

DNA repair refers to a collection of processes by which a cell identifies and corrects damage to the DNA molecules that encode its genome. In human cells, both normal metabolic activities and environmental factors such as UV light can cause DNA damage, resulting in as many as 1 million individual molecular lesions per cell per day. Many of these lesions cause structural damage to the DNA molecule and can alter or eliminate the cell's ability to transcribe the gene that the affected DNA encodes. Other lesions induce potentially harmful mutations in the cell's genome, which will affect the survival of its daughter cells after it undergoes mitosis. Consequently, the DNA repair process must be constantly active so it can respond rapidly to any damage in the DNA structure.

The rate of DNA repair is dependent on many factors, including the cell type, the age of the cell, and the extracellular environment. A cell that has accumulated a large amount of DNA damage, or one that no longer effectively repairs damage incurred by its DNA, can enter one of three possible states: an irreversible state of dormancy, known as senescence; cell suicide, also known as apoptosis or programmed cell death or unregulated cell division, which can lead to the formation of a tumor.

Tumor staging largely dictates the treatment and prognosis for NSCLC patients. Generally speaking, T1 and T2 tumors, are non-invasive and are treated with surgical resection in the case of stage I combined with chemoradiation for stage II tumors. 5-yr prognosis for stage I and stage II patients is 67% and 57%, respectively, who present with clean nodes. For patients with regional nodal involvement, 5 yr survival rates fall to 55% and 37%. With distal nodal involvement in later staged tumors, tumor recurrence and patient survival numbers suffer precipitously. Stage III tumors may receive neoadjuvant chemotherapy followed by surgical resection and then subsequent adjuvant chemotherapy with or without radiation. Stage IV tumors have metastasized to distal sites and care is considered palliative with a less than 1% of patients expected to survive 5 or more years. Approximately 170,000 new cases of NSCLC are diagnosed each year; 55% of these cases are localized (stage I, II, or III) while 45% are disseminated disease (stage IV) and are recommended for princally palliative care. It is the patient population that present with localized disease that are targeted for improved survival by tailoring therapeutic regimens to optimize for clinical endpoint parameters.

These tailored chemotherapeutic regimens that may or may not be combined with radiation can be placed into defined functional categories: (1) Microtubule disruting agents (Docetaxel Paclitaxel, Vinorelbine) (2) DNA adduct-forming platinating compounds (Carboplatin, Cisplatin, Oxaliplatin) (3) Base synthesis inhibitors (Pemetrexed) (4) Modified bases (gemicitibine) and (5) Tyrosine kinase inhibitors (cetuximab, gefitinib—used in tumors identified as EGFR driven). For more aggressive tumors, combinatorial approaches among several regimens may be undertaken. For instance, adenocarcinoma are indicated to benefit from microtubule disrupting agents in combination with platinating compound therapy. Squamous cell NSCLCs may benefit from combined platinating compounds in unison with gemcitibine. In addressing the combination of chemo and radiation with improved patient response and survival as the goal, a platinum response predictor could better inform the relative weight of chemotherapy in a multidisciplinary treatment regimen. A platinum response predictor has direct relevance due to the wide range of chemotherapies available.

The utility and need for molecular diagnostic tests are twofold relative to application to NSCLC management. First, patient populations are amalgams of many different global expression profiles. Kaplan-Meyer survival curves usually represent survival of a patient population following treatment with some therapeutic regimen. However, as each of the constituent patients' tumors within this population may be expected to have a distinct proteomic profile, one might expect that their responses to therapeutic regimens might segregate along specific expression patterns for defined sets of markers. Indeed separating the patient populations from Kaplan-Meyer survival curves based upon these defined discriminators into responders and non-responders may be imminently applicable in allowing physicians to prescribe a regimen with added survival benefit. Indeed, as chemotherapy by definition is toxic (poison), in some patient subgroupings, prescribing nothing over the chemotherapy may hold survival benefit.

Second, in clinical studies comprising drug discovery efficacy assessments a candidate therapeutic is tested against a patient population and then some level of response is measured. But, as noted above in Kaplan-Meyer survival curves, the patient population is an amalgam of many different molecular profile subtypes. Therefore, the likelihood that the patient population will respond universally to the candidate therapeutic is diminishing small. Such was the case with Iressa (gefitinib). A tyrosine kinase inhibitor targeting EGFR in NSCLC, clinical trials efficacy hovered around 15% response for the test population. Faced with the data of an 85% ineffective application to the total patient population, the drugs approval was called into question. Further research showed that that in patients comprising the 15% response group, that EGFR was modified (driver) for all patients within that group. The application of similar proteomic expression profiles will allow for better stratified patient selection and clinical trial design as well as more targeted and streamlined testing, approval and release of new therapeutics with real benefit to select individuals with the correct proteomic background.

DNA repair genes and/or proteins are already known to provide meaningful data relative to risk and prognosis, as well as offer valuable insights at decision inflection points for predictive therapeutic response in personalized medicine. While risk and prognostic significance should not be discounted, informations leading to correct decisions in patient management should prove most beneficial relative to actionable information leading to improved patient care and survival.

Multiple reports in the literature already indicate that DNA repair biomarker expression or lack thereof is strongly correlative patient subgroups more or less likely to respond to a specific prescribed chemotherapy. The rationale behind use of platinating compounds in combination with radioactivity is that the most actively dividing cells will incur the greatest DNA damage and thus suffer the most toxicity at the hands of the affecting agent(s). In theory, the cells that are most prone to induced lethality would have less efficient DNA repair mechanisms and those with efficient mechanisms would prove resistant to the agent of insult. While multiple DNA repair pathways are employed in cell maintenance, many of these already hold predicative value to chemotherapeutic susceptibility/resistance modeling studies relative to constituent biomarker expression profiles. Wang et al. (2009) and Kang et al. (2009) indicate that elevated APE1 and XRCC1, respectively, from the Base Excision Repair (BER) pathway correlate with cisplatin resistance. From the Mismatch Repair (MMR) pathway, Scartozzi et al (2006) indicate that low hMLH1 and hMSH2 correlates with cisplatin+gemcitibine resistance. Ceppi at al. (2009) indicate that low Polη from the Translesion Synthesis pathway (TLS) predicts greater sensitivity to cisplatin than high Polη. Multiple reports list ERCC1 from the Nucleotide Excision Repair (NER) Pathway as having both prognostic value to NSCLC patient survival as well as response to platinating, radiation, and taxane-inclusive chemoradiation therapeutics (Olaussen et al., 2006; Zheng et al., 2007; Lee et al., 2008; Gazdar, 2007; Wong et al., 2009; Holm, et al, 2009; Singh and Aggarwal, 2009). The Homologous Recombination (HR) Repair Pathway also has constituent enzymes such as RAD51 (Ko et al., 2008; Takenaka et al., 2007) and BRCA1 (Taron et al., 2004) whose expression levels when low are predictive of chemotherapeutic response and conversely when high are consistent with resistance.

Biomarkers of DNA repair may guide therapy because these are pathways that respond to damage to DNA caused by chemotherapy and radiation. Single pathways are not adequate to describe the DNA repair responses to even single chemotherapy agents for several reasons. First, chemotherapy causing DNA damage leads to many forms of DNA damage necessitating different biochemical mechanisms to repair. Second, the central DNA repair pathways can at least partially complement each other for cell survival. Even though the second pathway may not be as effective, it provides a salvage mechanism for the cell to return to cell division and growth. For a tumor cell, this is a mechanism for diminishing the strength of and/or evading cell cycle checkpoints.

### SUMMARY OF THE INVENTION

The present invention provides methods of predicting the response to chemotherapy and/or survivability of a subject having a non-small cell lung cancer by (a) measuring the level of an effective amount of one or more DNARMARKERS selected from DNARMARKERS 1-259 in a sample from the subject, and (b) comparing the level of the effective amount of the one or more DNARMARKERS to a reference value.

The present invention also provides methods of assessing the effectiveness or monitoring the treatment of a subject with non-small cell lung cancer. In one embodiment, the method of accessing the effectiveness of a treatment of a subject with cancer includes (a) measuring the level of an effective amount of one or more ok DNARMARKERS selected from DNARMARKERS 1-259 in a sample from the subject, and (b) comparing the level of the effective amount of the one or more DNARMARKERS to a reference value. In another embodiment, the method of monitoring the treatment of a subject with cancer includes (a) detecting the level of an effective amount of one or more DNARMARKERS from DNARMARKERS 1-259 in a first sample from the subject at a first period of time, (b) detecting the level of an effective amount of one or more DNARMARKERS in a second sample from the subject at a second period of time, and (c) comparing the level of the effective amount of the one or more DNARMARKERS detected in step (a) to the amount detected in step (b), or to a reference value.

The present invention provides methods of determining the resistance or sensitivity of a cancer cell to a chemotherapeutic agent. In one embodiment, the method of determining the resistance of a cancer cell to a chemotherapeutic agent includes identifying a deficiency in a DNA Repair and DNA Damage Response Marker (i.e, DNARMARKER), where the absence of the deficiency indicates the cell is resistant to a chemotherapeutic agent In another embodiment, the method of determining the sensitivity of a cancer cell to a chemotherapeutic agent includes identifying a deficiency in a DNA Repair and DNA Damage Response Marker, where the presence of the deficiency indicates the cell is sensitive to a chemotherapeutic agent.

The DNARMARKER is selected from Table 2. Optionally, one or more additional DNARMARKERS from Table 1 are detected. Preferably, the DNARMARKER is XPF, FANCD2, pMK2, PAR, p53, ERCC1, ATM, MLH1, PARP1, pH2AX, pHSP27, BRCA1, BRCA2, RAD51, NQO1, or MSH2. In some embodiments the DNARMARKER is MSH2, p53, pMK2(n), pMK2(c=n), or ATM. In other embodiments the DNARMARKER is MSH2, p53, or ATM. In further embodiments the DNARMARKER is p53, pMK2 ERCC1, PARP1 or ATM.

The DNARMARKER of interest is selected from Table 3. Optionally, one or more additional DNARMARKERS from Tables 1 and/or 2 are detected.

The DNARMARKER of interest is selected from Table 4, herein detected by amplification or deletion at the level of the gene. Such copy number variation can be determined via a variety of techniques including but not limited to fluorescence in situ hybridization (FISH), chromogenic in situ hybridization (CISH), next generation quantitative DNA sequenceing, quantitative in situ PCR. Optionally, one or more additional DNARMARKERS from Tables 1, 2 and/or 3 are detected, or any combination of markers can be utilized.

The chemotherapeutic agent is a platinating agent (i.e carboplatin, oxaliplatin, cisplatin), taxane or both. Optionally, the chemotherapeutic agent is chemoradiotherapy.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIG. 1. FFPE non-small cell lung cancer sectioned tissue stained with DNA repair biomarker POLη. 10 um thick sections from three separate patients' tumors were incubated with mouse monoclonal primary Ab (clone B-7 from Santa Cruz Biotechnology) and then HRP-labeled secondary Ab and detected via DAB staining. Typical strong, moderate, and negative stains are depicted from representative areas in each of the panels derived from the 3 differentially stained tumors.

FIG. 2. FFPE non-small cell lung cancer sectioned tissue stained with DNA repair biomarker RAD51 10 um thick sections from three separate patients' tumors were incubated with rabbit polyclonal primary Ab (Millipore) and then HRP-labeled secondary Ab and detected via DAB staining. Typical strong/moderate, weak, and negative stains are depicted from representative areas in each of the panels derived from the 3 differentially stained tumors.

FIG. 3. Dynamic range of expression for 3 DNA repair biomarkers (FancD2, ERCC1, and RAD51) from commercial TMA-derived non-small cell lung cancer samples. TMA LUC1503 (Pantomics) containing 75 non-small cell lung cancer patient samples in duplicate was stained for the specific marker of interest. Slides/cores were imaged and scored via Aperio Scanscope and an algorithm applied to give added weight to samples with a higher percentage of cells scoring as 2+ and 3+ (QIM). Two cores for each patient were averaged and then ordered by increasing QIM score to generate the depicted distribution plots (shown in both linear and log scales).

FIG. 4. RAD51, ERCC1, and FancD2 QIM percent difference relative to the mean for all stained samples from a commercial non-small cell lung cancer TMA. QIM scores for RAD51, ERCC1, and FancD2 IHC stains from commercial LUC1503 (Pantomics) depicted in FIG. 3 were averaged and then individual QIM scores charted as a percentage difference relative to that mean. This plot depicts substantially greater or lesser differences in expression relative to the overall mean. For patients that score at or close to 0, QIM scores relative to the mean cannot go lower than 100% by definition.

FIG. 5. RAD51 and FancD2 expression co-vary among patient samples on a commercial non-small cell lung cancer TMA. Data from FIG. 3 for LUC1503 TMA was examined more closely for patterns of expression. For samples in which RAD51 biomarker expression varied by greater than 10% relative to the mean (50 of the 75 samples on the TMA), FancD2 expression was tightly correlated in all cases. Alternatively, ERCC1 expression was independent of RAD51/FancD2 correlated levels.

FIG. 6. Dynamic range of expression for 2 additional DNA repair biomarkers (XPF and ATM) from commercial TMA-derived non-small cell lung cancer samples. TMA LC242 (Biomax) containing 20 non-small cell lung cancer patient samples was stained for the specific marker of interest. Slides/cores were imaged and scored via Aperio Scanscope and an algorithm applied to give added weight to samples with a higher percentage of cells scoring as 2+ and 3+ (QIM). Each patient was scored and ordered by increasing QIM value to generate the depicted distribution plots (shown in both linear and log scales).

FIG. 7. XPF and ATM QIM percent difference relative to the mean for all stained samples from a commercial non-small cell lung cancer TMA. QIM scores for XPF and ATM IHC stains from commercial TMA LC242 (Biomax) depicted in FIG. 6 were averaged and then individual QIM scores charted as a percentage difference relative to that mean. This plot depicts ATM for patients 4 and 5 as being substantially greater than the overall mean while XPF is substantially higher than the mean for patients 7 and 8. For patients that score at or close to 0, QIM scores relative to the mean cannot go lower than 100% by definition.

FIG. 8. Dynamic range and reproducibility expression plots for DNA repair biomarkers (FancD2 and ERCC1) from TMA-derived non-small cell lung cancer samples. Dynamic range expression plots for ERCC1 and FancD2 from FIG. 3 are shown alongside the range of expression for an n=2 for each of the 75 patients to indicate typical sample to sample QIM variability for samples derived from individual patients. Such small variability (in most cases usually less than 20%) may arise due to technical (i.e differential fixation) or biological (tumor heterogeneity) phenomenon.

FIG. 9. Dynamic biomarker expression for XPF, FancD2 and Polη among a cohort of non-small cell lung cancer FFPE-sectioned stage IIIa and stage IIIb biopsy or resected tumor samples. A TMA containing biopsy or surgical resections were assayed for biomarker expression for XPF, FancD2, and Polη. Forty-eight patients with discernable tumor were scored and ordered on the indicated plots indicating a broad dynamic range among samples comprising the cohort. Also shown is an ordered plot for average QIM for all three biomarkers indicting that these markers can be combined and averaged to develop algorithms which may preserve, enhance and add predictive and/or prognostic value to the resulting data sets.

FIG. 10. Individual patient QIM scores for three DNA repair biomarkers (XPF, FancD2, and Polη). The figure depicts a bar graph of QIM scores for each of three biomarkers tested on the TMA comprising a cohort of stage IIIa and stage IIIb non-small cell lung cancer patients (also shown in FIG. 9). This bar graph indicates that each of the patient samples has a distinct profile for each of the markers tested from other samples within the cohort. Certainly direct correlations may arise between separate biomarkers while some profiles may be anti-thetical with other biomarkers. The key information here is that the biological panel for each sample will be non-identical for most samples, thus providing a continuum against to measure clinical endpoint data.

FIG. 11. NSCLC tumor heterogeneity for XPF expression. Three separate fields of view from 3 different areas f a single tumor were stained for XPF. The resulting panels show differential staining of XPF of non-small cell lung cancer cells in distinct and separate regions of the tumor. Such staining is consistent with a road and dynamic range of expression for XPF (even within a single tumor) and argues for tumor cell heterogeneity with respect to dynamic biomarker expression patterns.

FIG. 12. Individual patient Q-score distribution for ATM. The figure depicts a Q-score for a commercially available NCSLC TMA compared with the disctribution of Q-scores for all patients for a given biomarker.

FIG. 13. Individual patient Q-score distribution for MSH2. The figure depicts a Q-score for a commercially available NCSLC TMA compared with the disctribution of Q-scores for all patients for a given biomarker.

FIG. 14. Individual patient Q-score distribution for BRAC-1. The figure depicts a Q-score for a commercially available NCSLC TMA compared with the disctribution of Q-scores for all patients for a given biomarker.

FIG. 15. Individual patient Q-score distribution for ERCC1. The figure depicts a Q-score for a commercially available NCSLC TMA compared with the disctribution of Q-scores for all patients for a given biomarker.

FIG. 16. Individual patient Q-score distribution for SNRNP70. The figure depicts a Q-score for a commercially available NCSLC TMA compared with the disctribution of Q-scores for all patients for a given biomarker.

FIG. 17. Individual patient Q-score distribution for XPF. The figure depicts a Q-score for a commercially available NCSLC TMA compared with the disctribution of Q-scores for all patients for a given biomarker.

FIG. 18. Individual patient Q-score distribution for PARP-1. The figure depicts a Q-score for a commercially available NCSLC TMA compared with the disctribution of Q-scores for all patients for a given biomarker.

FIG. 19. Individual patient Q-score distribution for p53. The figure depicts a Q-score for a commercially available NCSLC TMA compared with the disctribution of Q-scores for all patients for a given biomarker.

FIG. 20. Individual patient Q-score distribution for pMK2. The figure depicts a Q-score for a commercially available NCSLC TMA compared with the disctribution of Q-scores for all patients for a given biomarker.

### DETAILED DESCRIPTION OF THE INVENTION

The invention relates to the observation that tumor cells have altered DNA repair and DNA damage response pathways and that loss of one of these pathways renders the cancer more sensitive to a particular class of DNA damaging agents. Cancer therapy procedures such as chemotherapy and radiotherapy work by overwhelming the capacity of the cell to repair DNA damage, resulting in cell death.

There are six major DNA repair pathways distinguishable by several criteria which can be divided into three groups those that repair single strand damage and those that repair double stand damage. Single stranded damage repair pathways include Base-Excision Repair (BER); Nucleotide Excision Repair (NER); Mismatch Repair (MMR); Homologous Recombination/Fanconi Anemia pathway (HR/FA); Non-Homologous Endjoining (NHEJ), and Translesion DNA Synthesis repair (TLS).

BER, NER and MMR repair single strand DNA damage. When only one of the two strands of a double helix has a defect, the other strand can be used as a template to guide the correction of the damaged strand. In order to repair damage to one of the two paired molecules of DNA, there exist a number of excision repair mechanisms that remove the damaged nucleotide and replace it with an undamaged nucleotide complementary to that found in the undamaged DNA strand. BER repairs damage due to a single nucleotide caused by oxidation, alkylation, hydrolysis, or deamination. NER repairs damage affecting longer strands of 2-30 bases. This process recognizes bulky, helix-distorting changes such as thymine dimers as well as single-strand breaks (repaired with enzymes such UvrABC endonuclease). A specialized form of NER known as Transcription-Coupled Repair (TCR) deploys high-priority NER repair enzymes to genes that are being actively transcribed. MMR corrects errors of DNA replication and recombination that result in mispaired nucleotides following DNA replication.

NHEJ and HR repair double stranded DNA damage. Double stranded damage is particularly hazardous to dividing cells. The NHEJ pathway operates when the cell has not yet replicated the region of DNA on which the lesion has occurred. The process directly joins the two ends of the broken DNA strands without a template, losing sequence information in the process. Thus, this repair mechanism is necessarily mutagenic. However, if the cell is not dividing and has not replicated its DNA, the NHEJ pathway is the cell's only option. NHEJ relies on chance pairings, or microhomologies, between the single-stranded tails of the two DNA fragments to be joined. There are multiple independent “failsafe” pathways for NHEJ in higher eukaryotes. Recombinational repair requires the presence of an identical or nearly identical sequence to be used as a template for repair of the break. The enzymatic machinery responsible for this repair process is nearly identical to the machinery responsible for chromosomal crossover during meiosis. This pathway allows a damaged chromosome to be repaired using the newly created sister chromatid as a template, i.e. an identical copy that is also linked to the damaged region via the centromere. Double-stranded breaks repaired by this mechanism are usually caused by the replication machinery attempting to synthesize across a single-strand break or unrepaired lesion, both of which result in collapse of the replication fork.

Translesion synthesis is an error-prone bypass method where a DNA lesion is left unrepaired during S phase, and is repaired later in the cell cycle. The DNA replication machinery cannot continue replicating past a site of DNA damage, so the advancing replication fork will stall on encountering a damaged base. The translesion synthesis pathway is mediated by specific DNA polymerases that insert alternative bases at the site of damage and thus allow replication to bypass the damaged base to continue with chromosome duplication. The bases inserted by the translesion synthesis machinery are template-independent, but not arbitrary; for example, one human polymerase inserts adenine bases when synthesizing past a thymine dimer. If this residue is not repaired at a later step, the process is mutagenic.

Cancer cells accumulate high levels of DNA damage. This damage may result from their heightened proliferative activity or from exposure to chemotherapy or ionizing radiation.

One trait that is desirable in the study of biomarker expression and aligning expression with response is a dynamic level by which to measure the marker or panel of markers against clinical endpoints. Clinical endpoints include for example, disease free survival or overall survival.

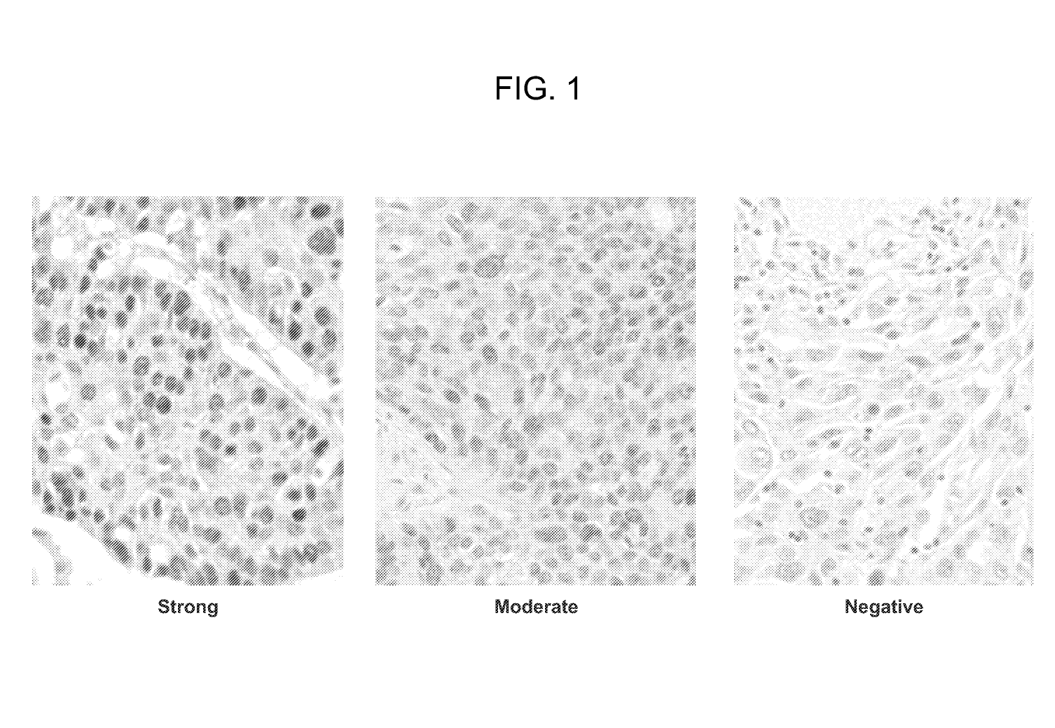

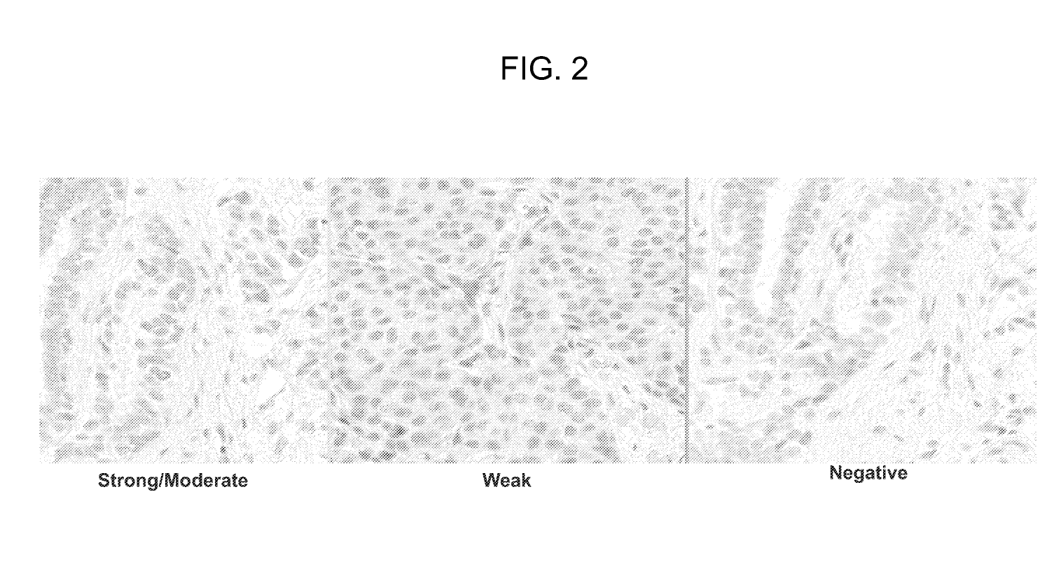

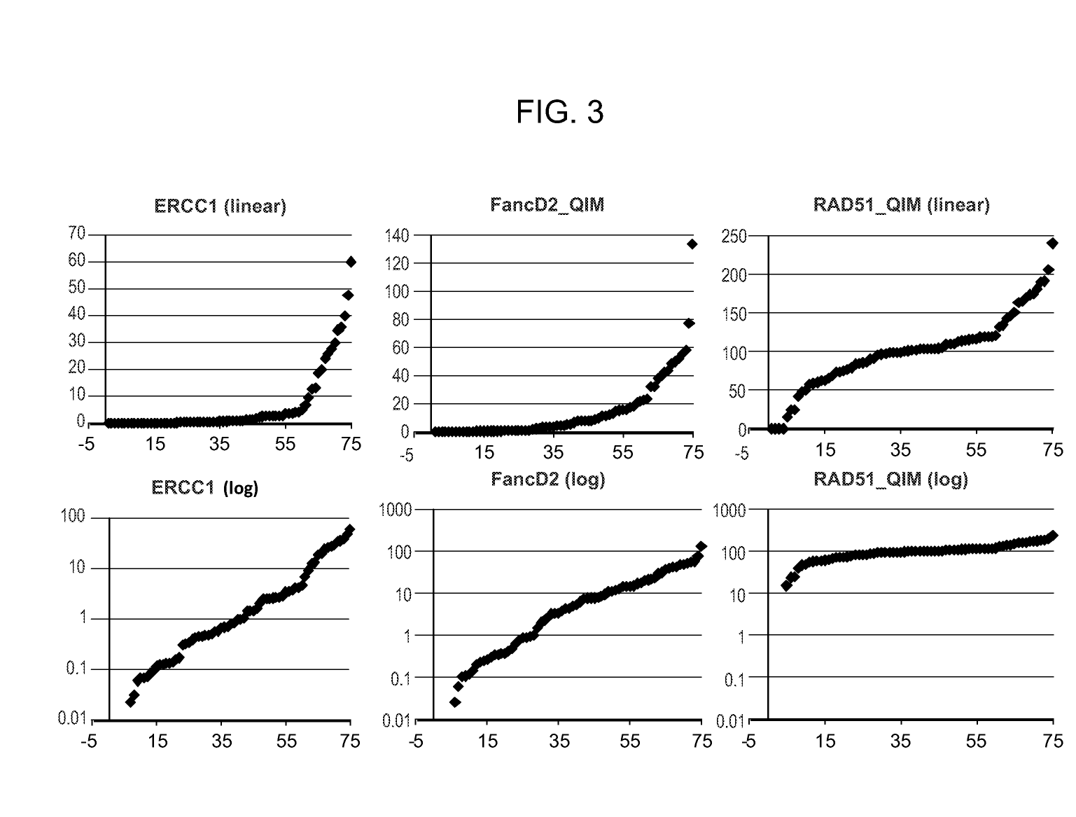

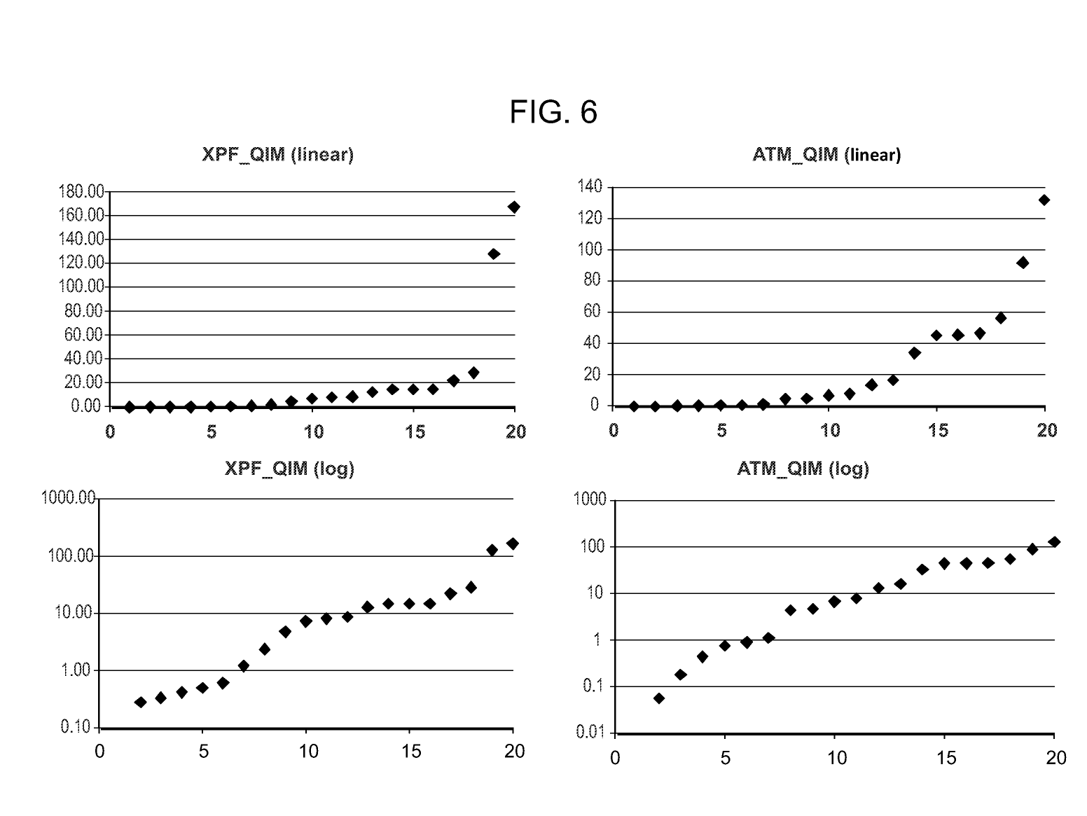

FIGS. 1 and 2 depict IHC staining from FFPE NSCLC sections varying in expression levels for 3 separate patients for Polη and RAD51, respectively. The expression levels can be determined and further quantified by defining algorithms for cells staining as 1+, 2+, and 3+ along with the percent of unstained cells marked as 0. Further verticality can be added to these scores by weighting the percent of cells scoring as 2+ and still weighted further for 3+ to generate a QIM score. FIGS. 3 and 6 illustrate these weighted scores (ranked in order of increasing QIM score) derived from staining of a patient population. In FIG. 3, 75 patients from a commercial TMA were stained and scored for ERCC1, FancD2, and RAD51. For both ERCC1 and FancD2, the patient group appears to reflect a logarithmic pattern while for RAD51, expression adopts a more linear pattern. In FIG. 6, a commercial TMA with 20 patients indicates distribution for XPF and ATM. Again, the expression pattern in this very small patient population is consistent with a logarithmic QIM score distribution.

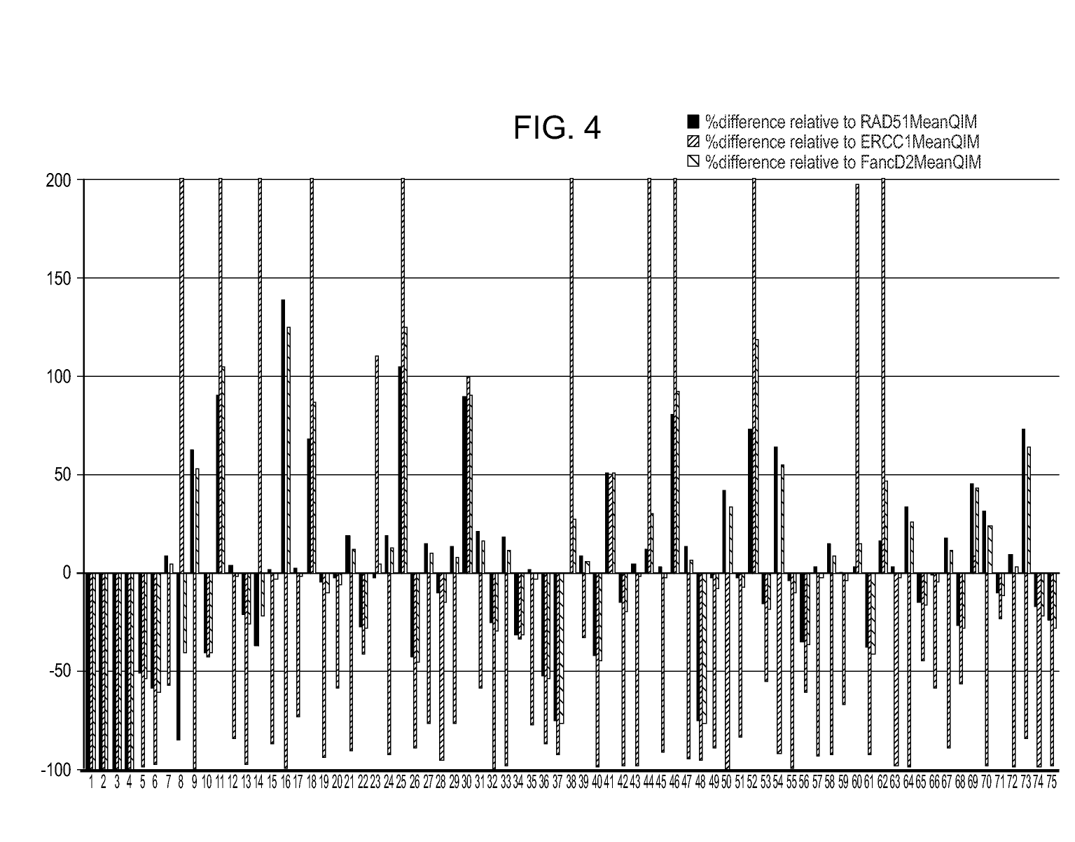

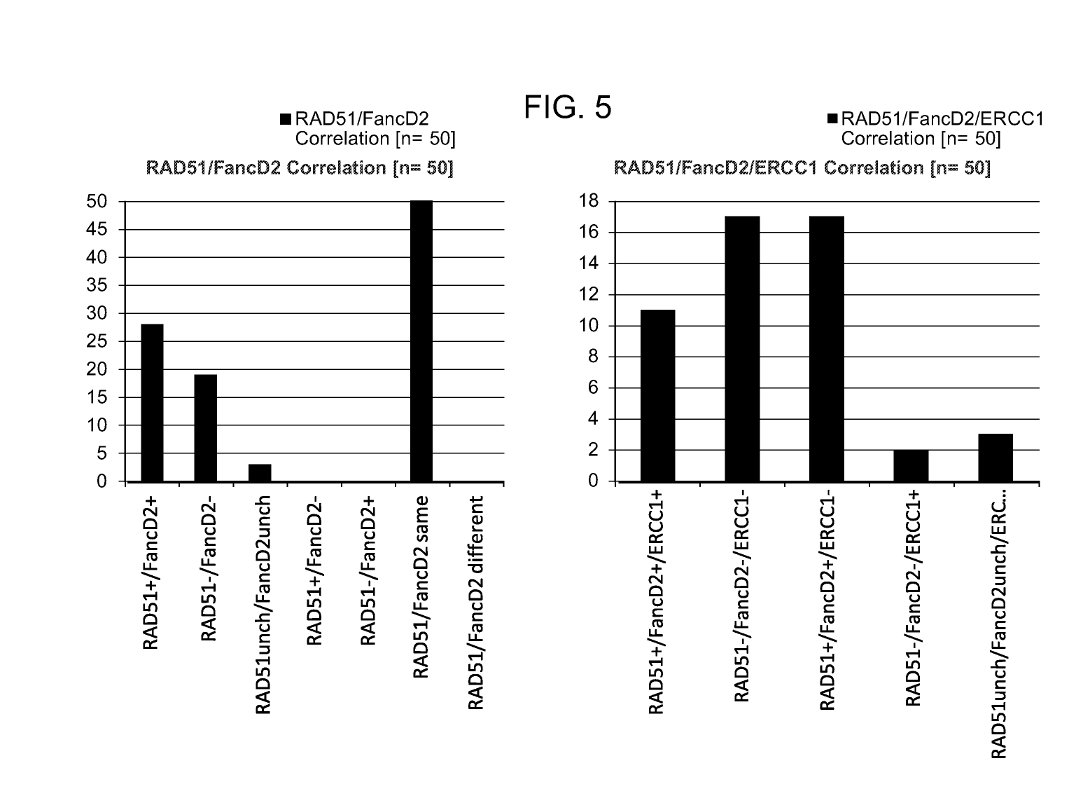

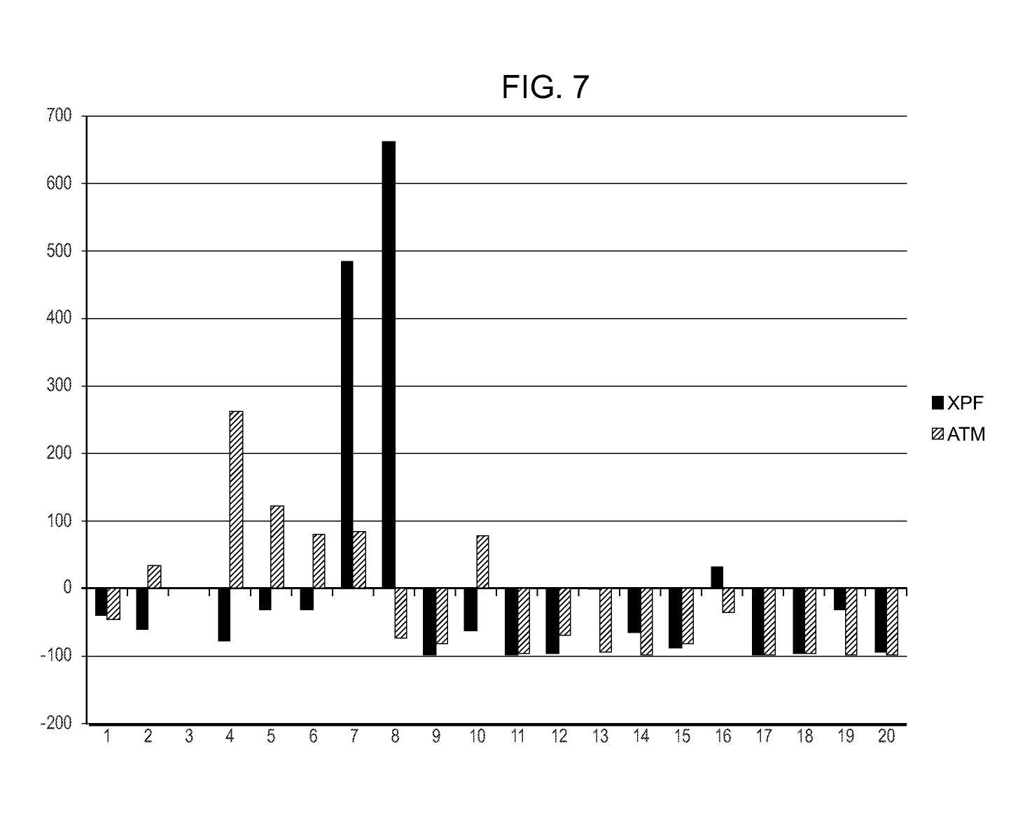

The data from FIG. 3 was averaged for each biomarker and then the represented in FIG. 4 as percent change relative to the mean for RAD51, ERCC1, and FancD2. Please note that for samples with QIM scores approaching 0 (unstained), that you cannot achieve less than 100% relative to the mean, while the lower the mean, the greater potential for higher percentage increases relative to the mean. In this graph, increases relative to the mean have been capped at 200% (a 3 fold increase). Data from this figure was utilized in FIG. 5 to show that for each of the samples in which ERCC1 QIM differed by greater than 10% relative to the mean, that RAD51 and FancD2 levels were tightly correlated in all 50 samples. In contract, ERCC1 variation relative to the mean was independent of RAD51/FancD2 variation relative to the mean for this group of patients. Similarly, FIG. 7 shows ATM and XPF QIM data from FIG. 6 distribution plots for XPF and ATM charted as percent increase/decrease relative to overall mean. The data indicate samples that are dramatically different relative to the mean although the patient population size from this TMA is too small to assign correlation substanitiveness.

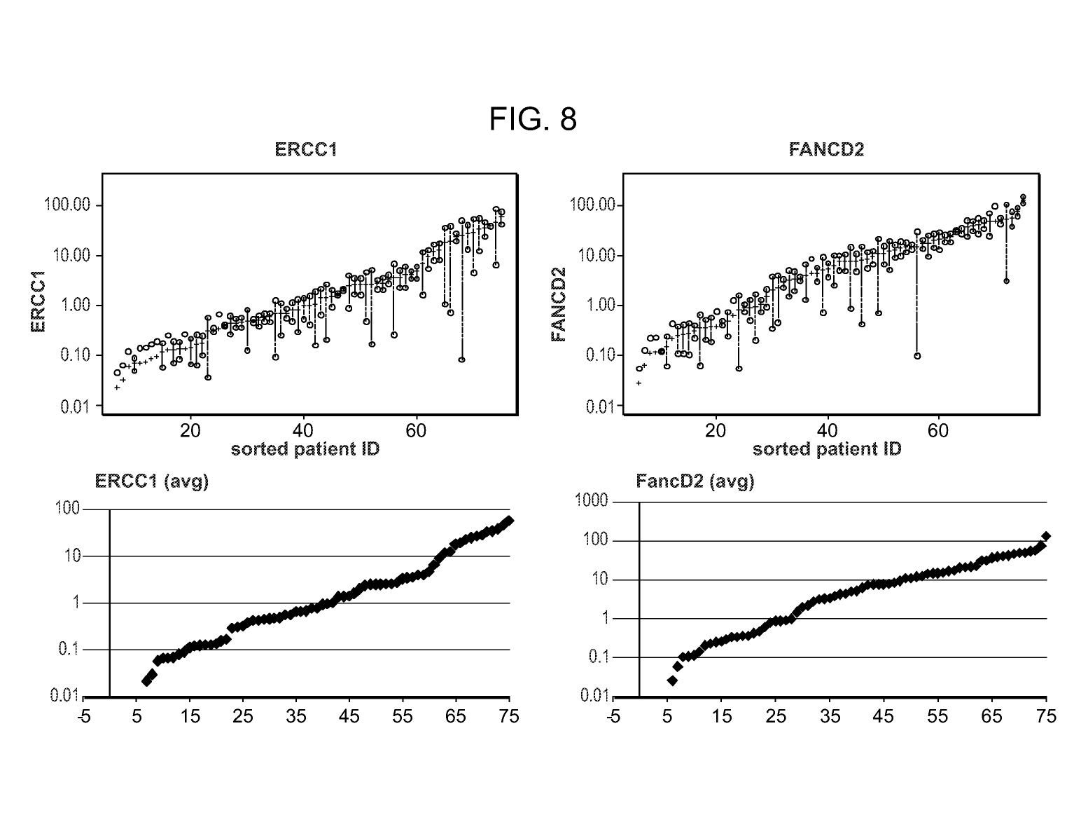

Within any one patient, the stain for a given biomarker will not be uniform and the overall intensity of the sample (and QIM score 0 will be based on a cumulative continuum of all stained cells. This continuum can be influenced by several criteria including tumor heterogeneity. FIG. 8 indicates the average QIM scores generated in FIG. 3 for ERCC1 as well as FancD2 for a commercial TMA. The average was generated from the scoring of 2 cores each and the 2 scores as well as the ordered average distribution are indicated. For most patients, the range of expression does not exceed 20% difference around the QIM average.

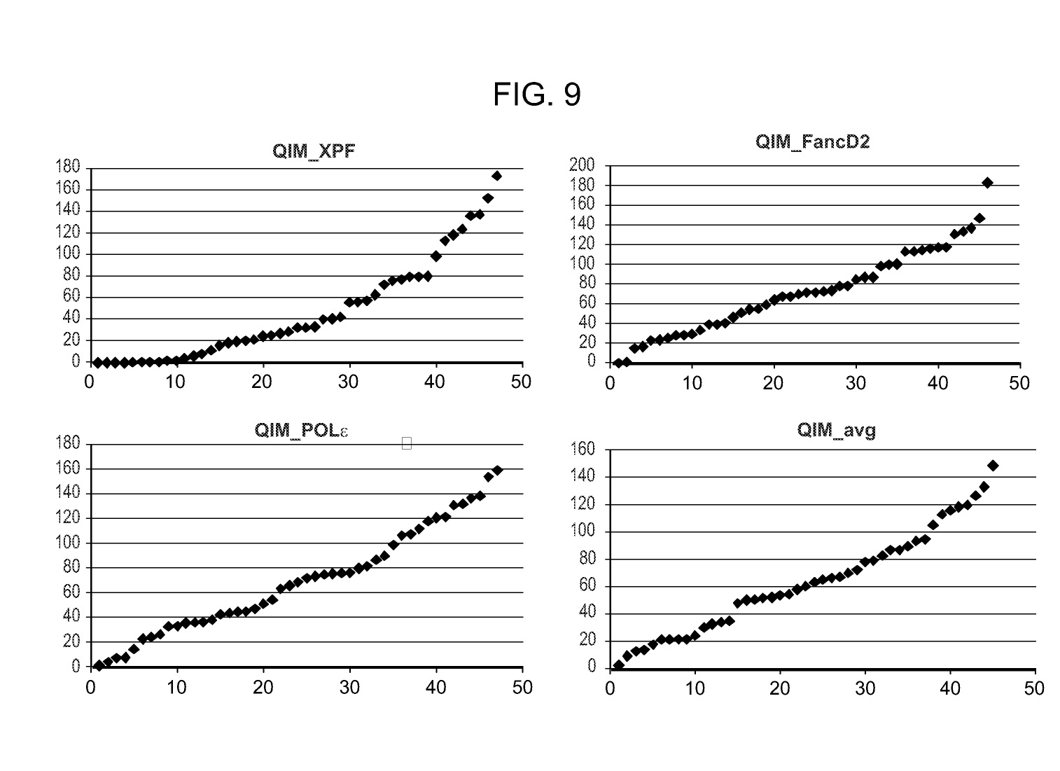

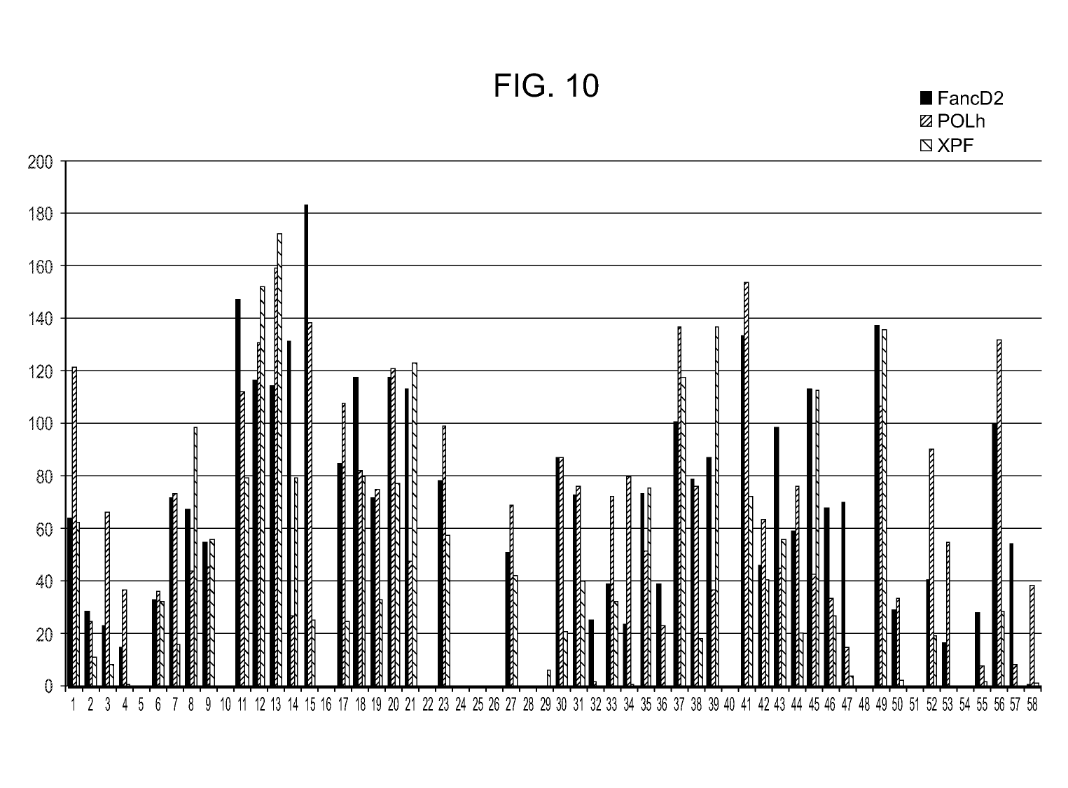

FIGS. 9 and 10 show distribution plots for QIM scores derived from biomarker staining of a group of stage IIIa and stage IIIb NSCLC biopsy and resected tumors prior to treatment with platinating compounds and/or taxanes. Across a group of 48 patients with discernable tumor, XPF, FancD2, and POLη all display a broad dynamic range of expression, as does the overall mean, average for each individual patient. When examining individual patients, XPF, FancD2, and POLη largely are expressed at levels independent of one another (FIG. 10). Multiple layers of independently acting variable may allow for finer stratification relative to clinical endpoint parameters.

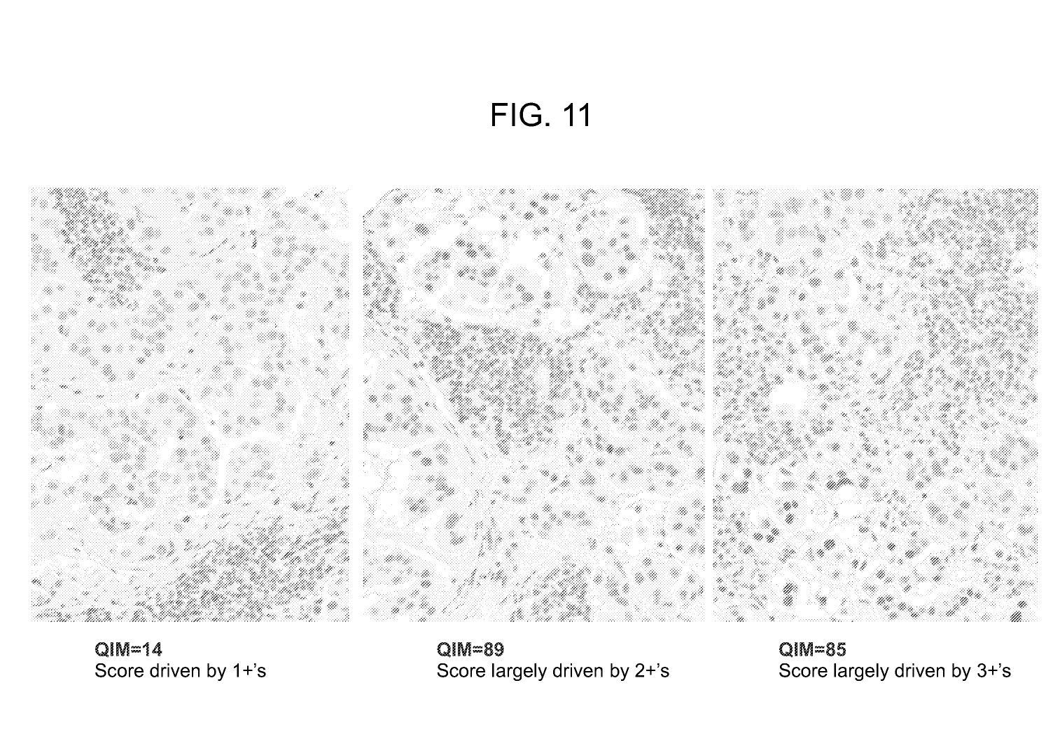

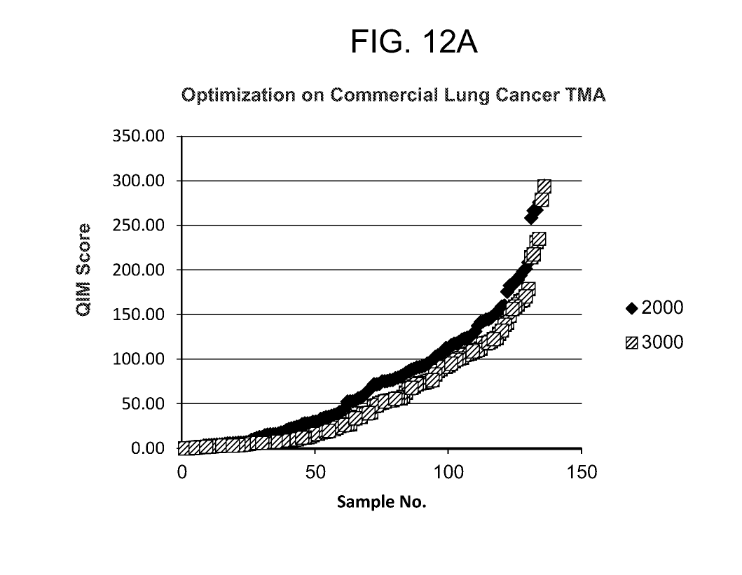

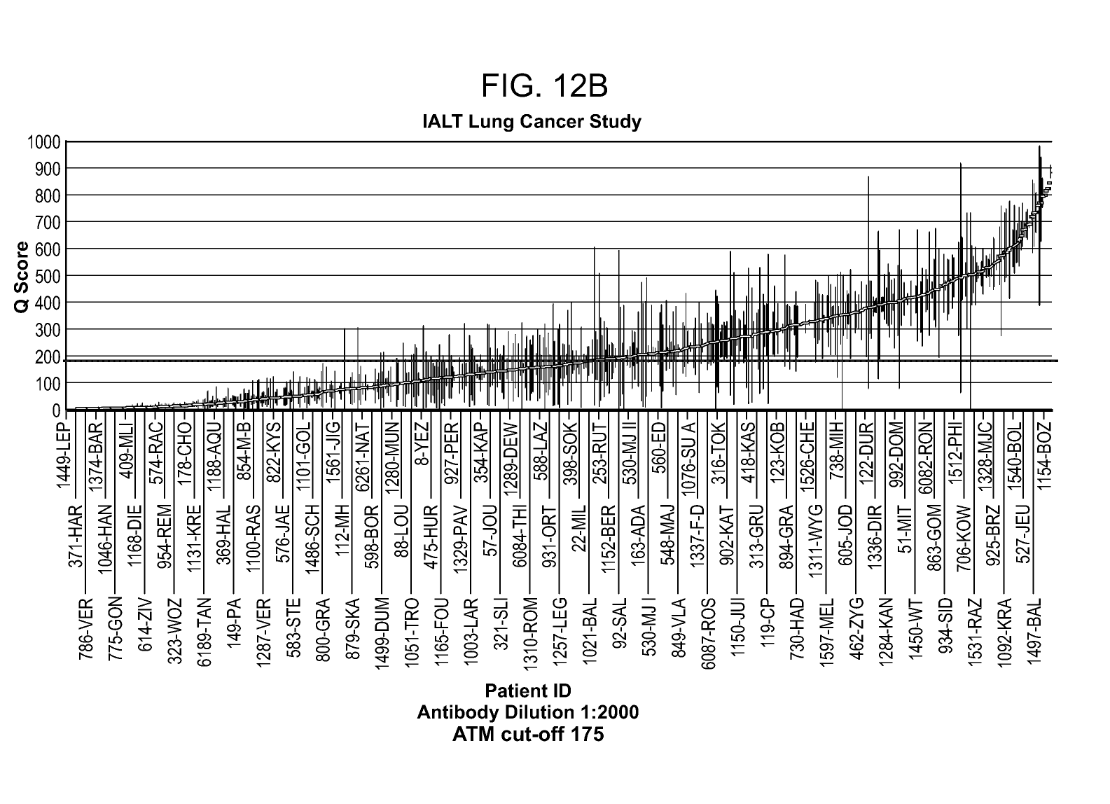

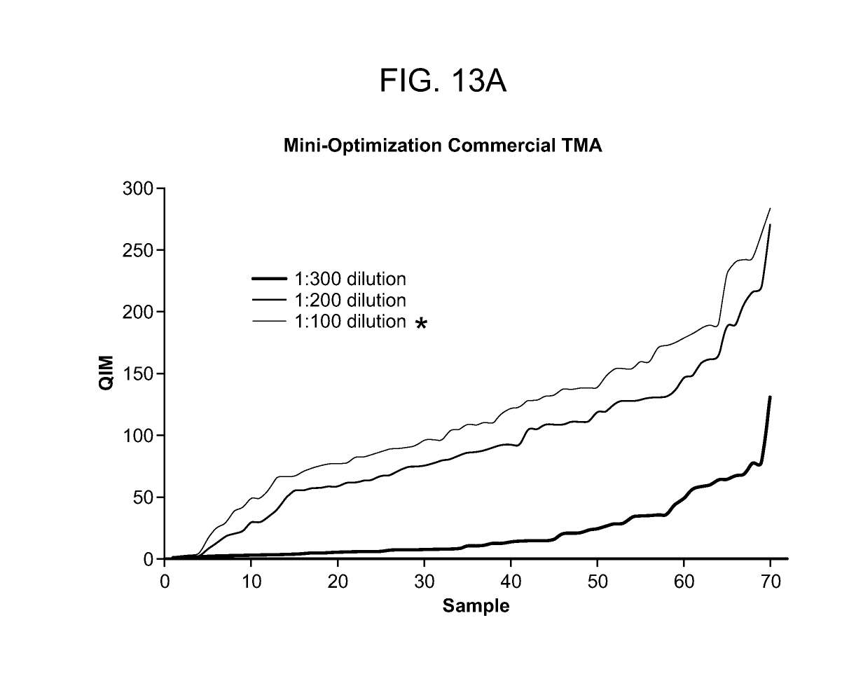

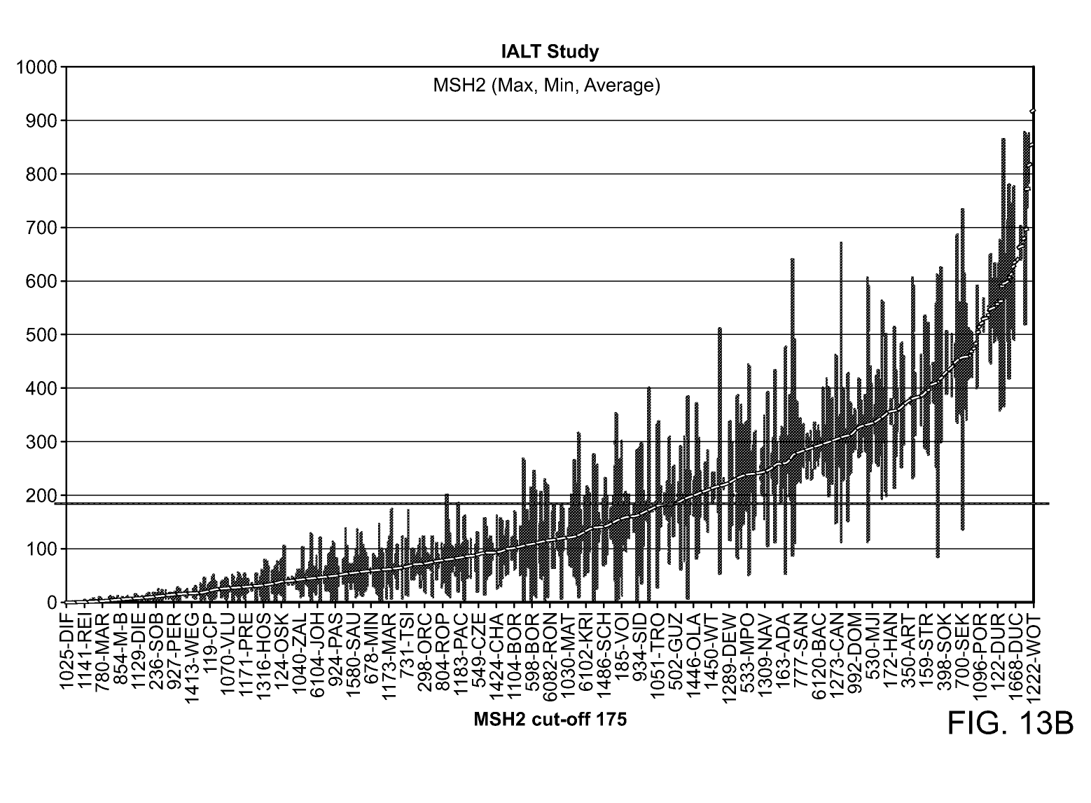

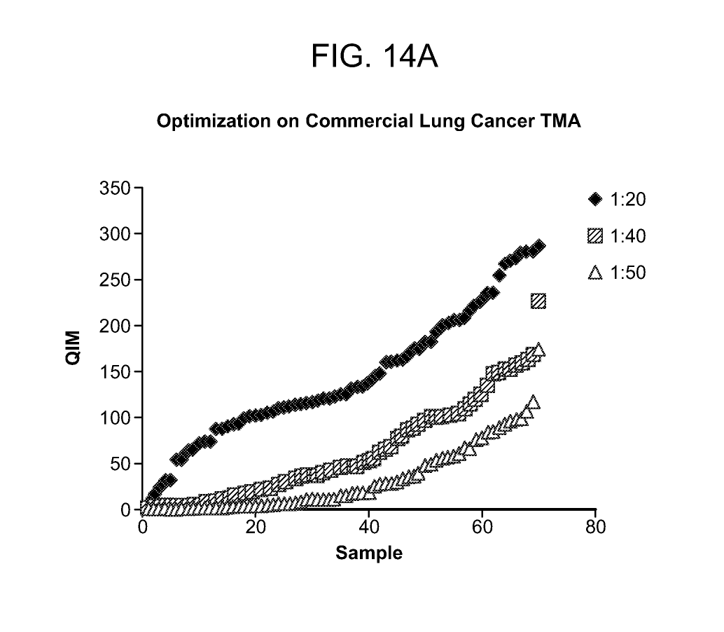

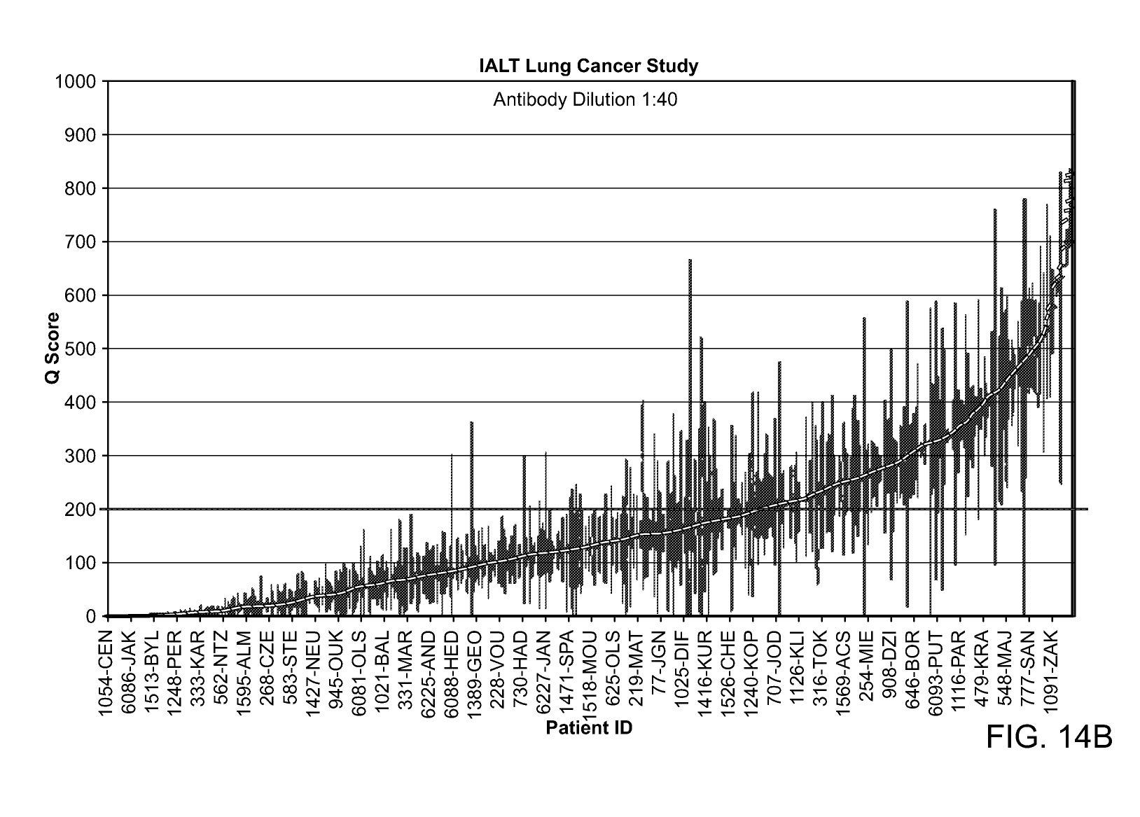

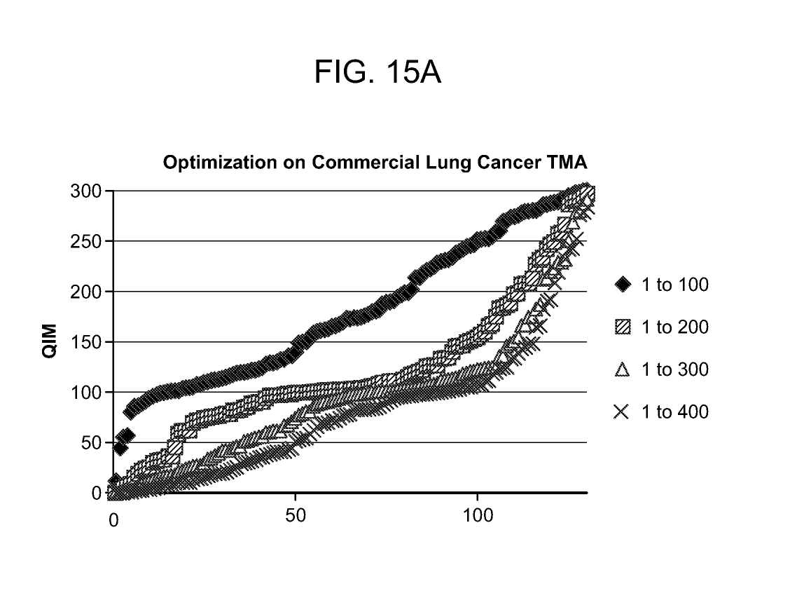

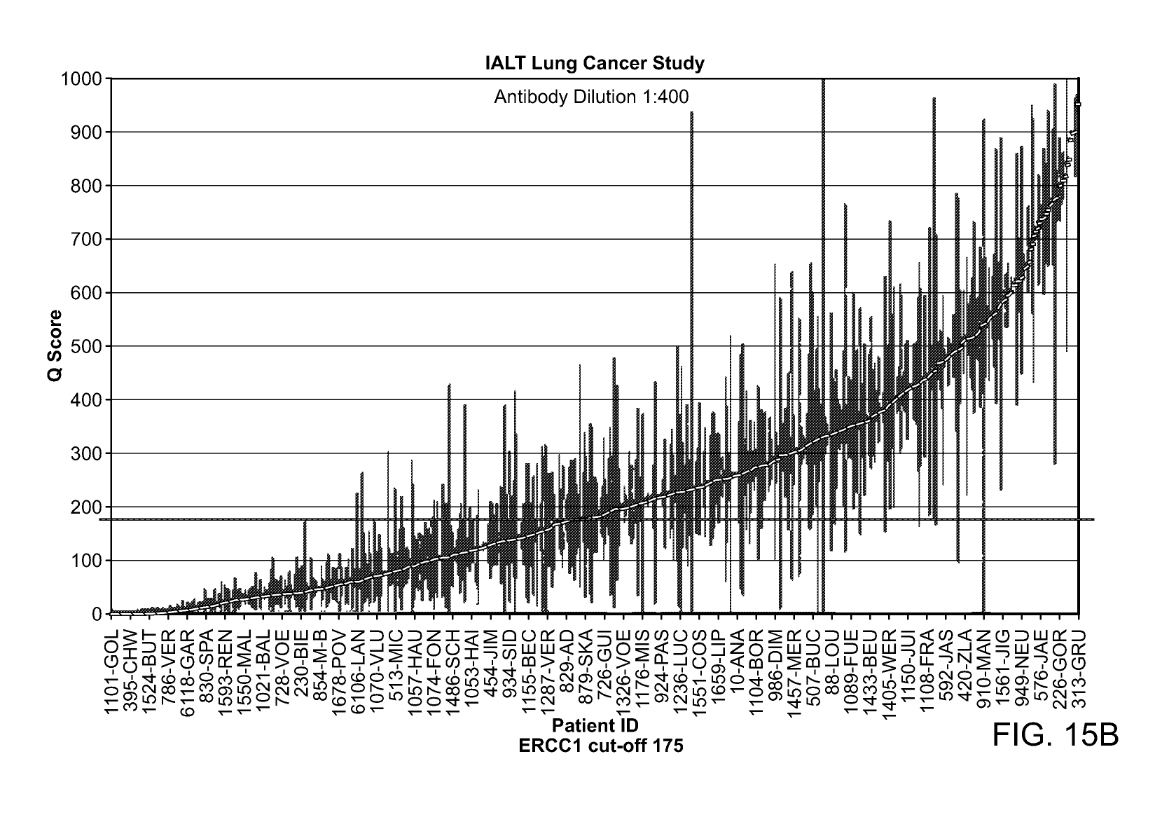

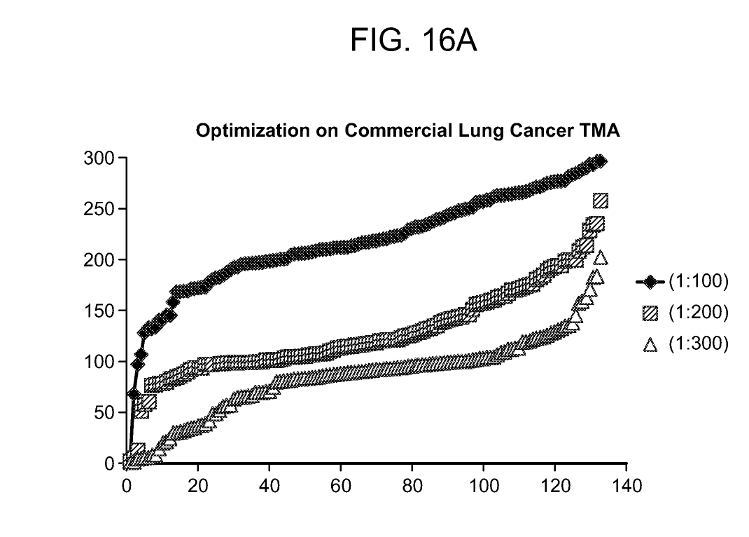

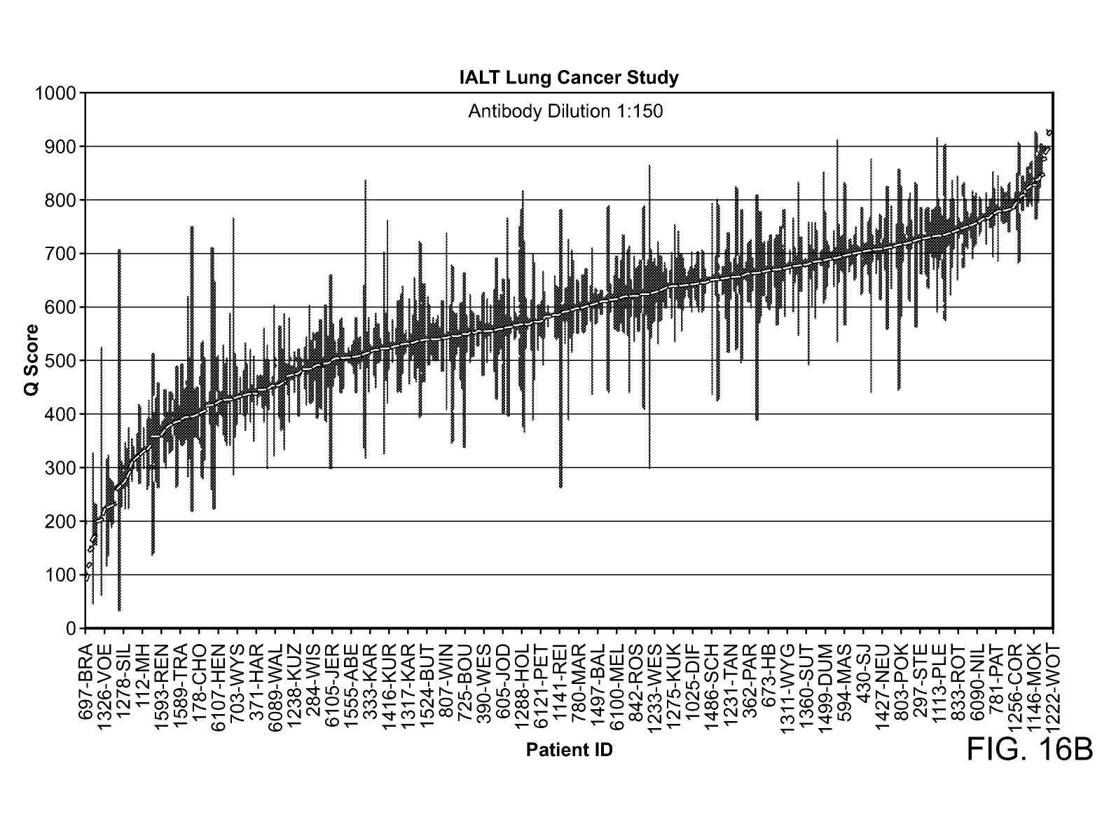

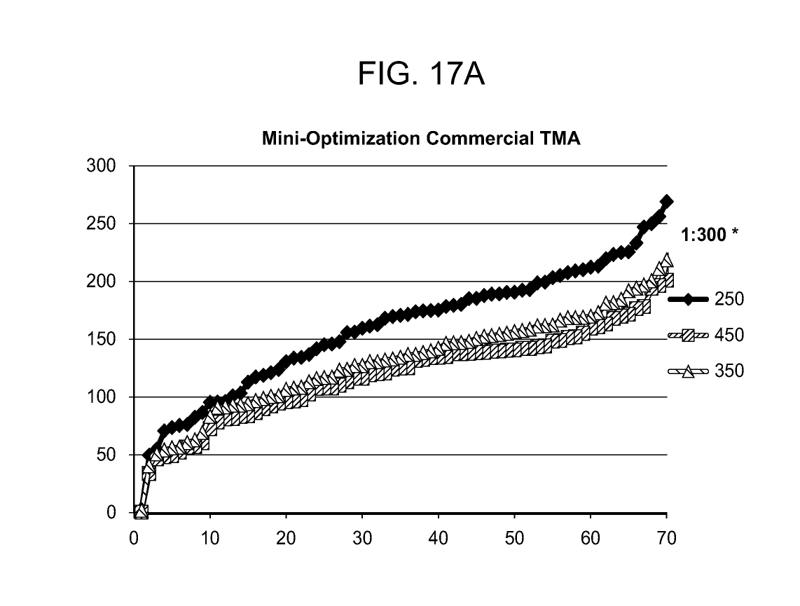

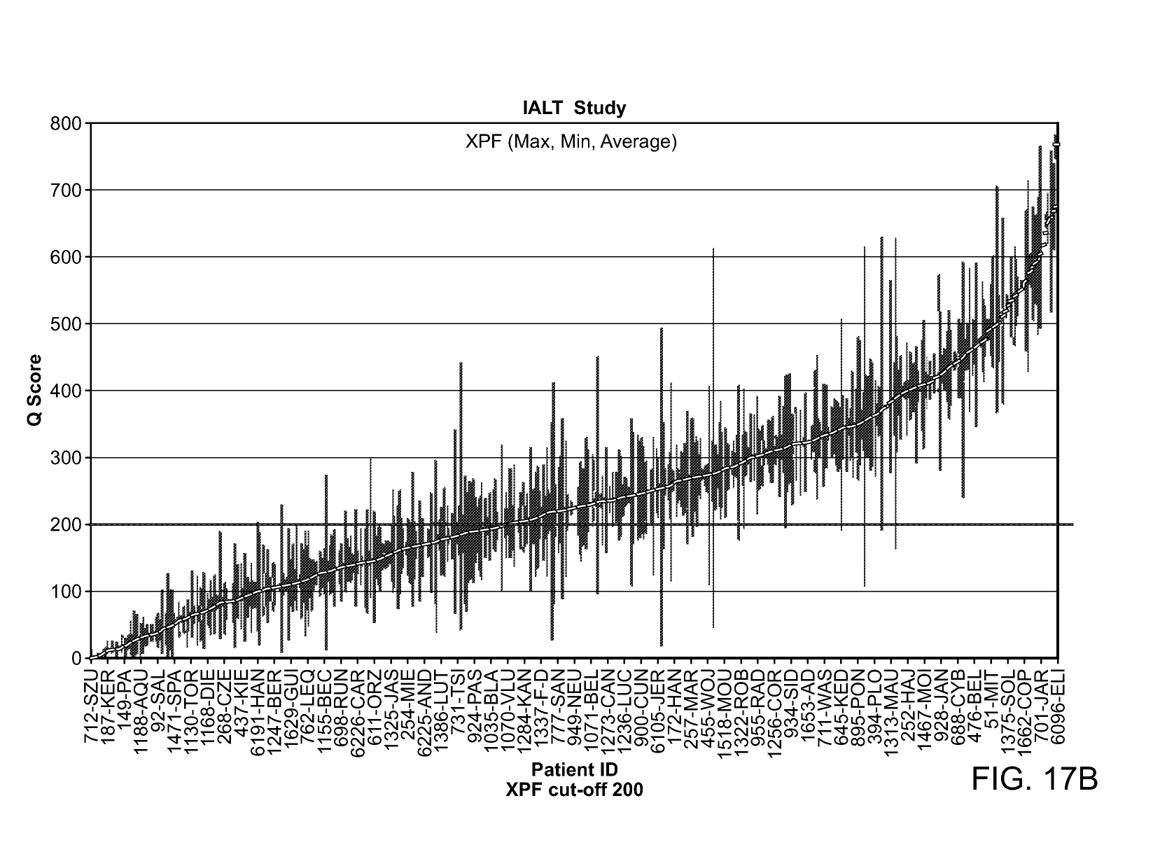

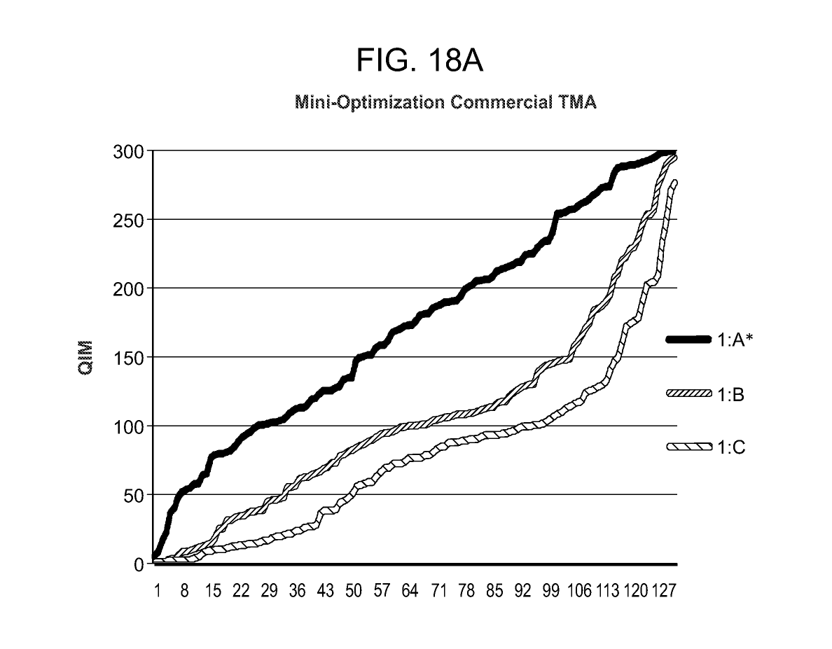

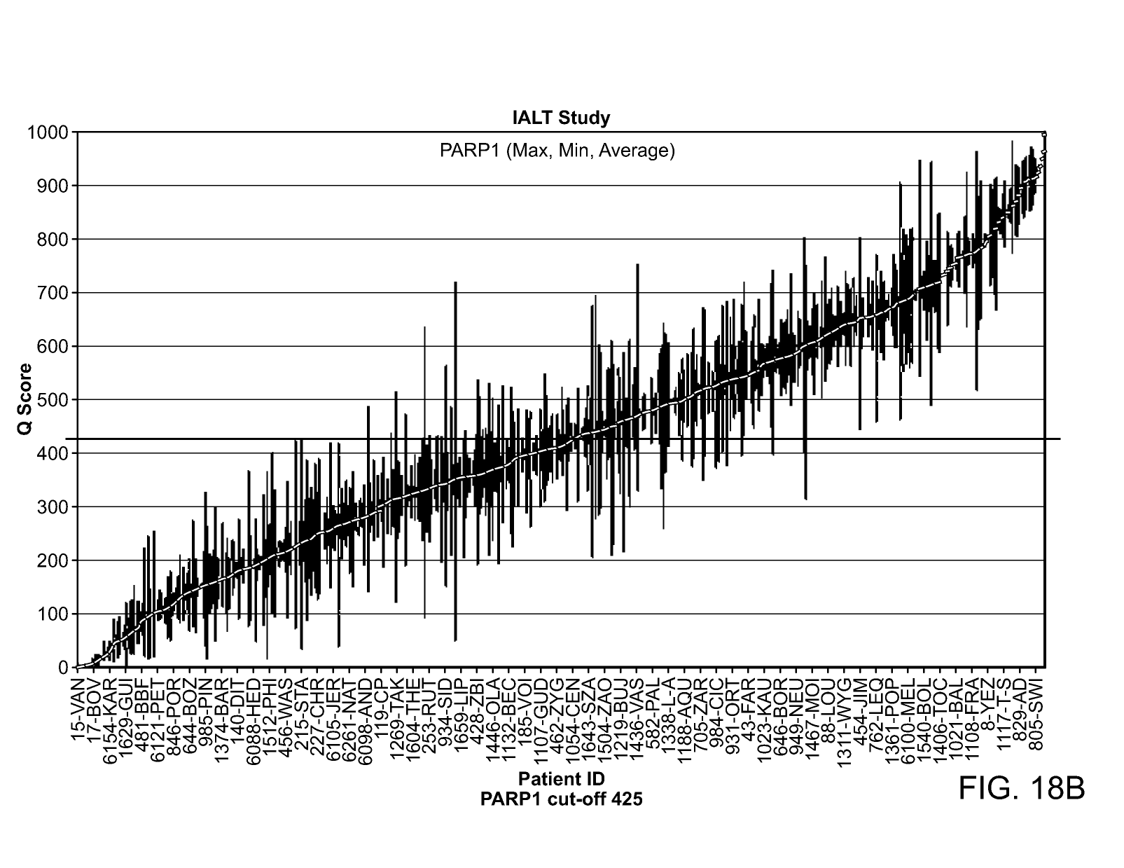

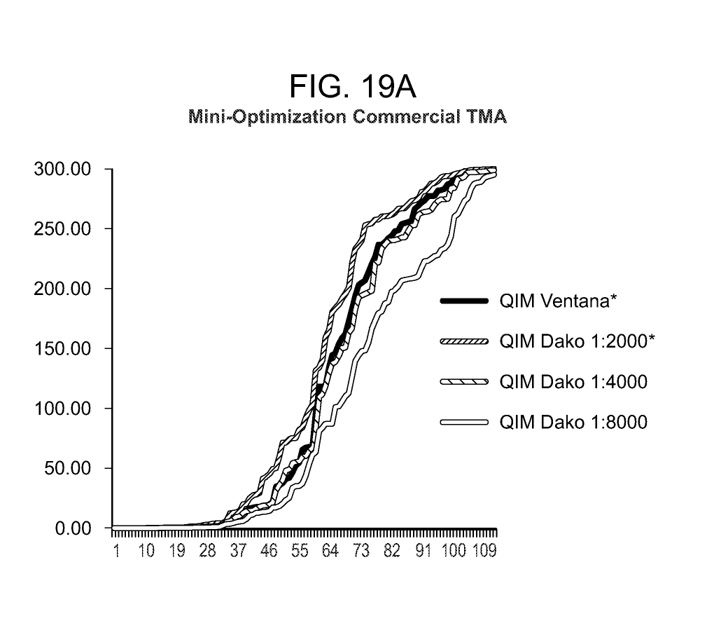

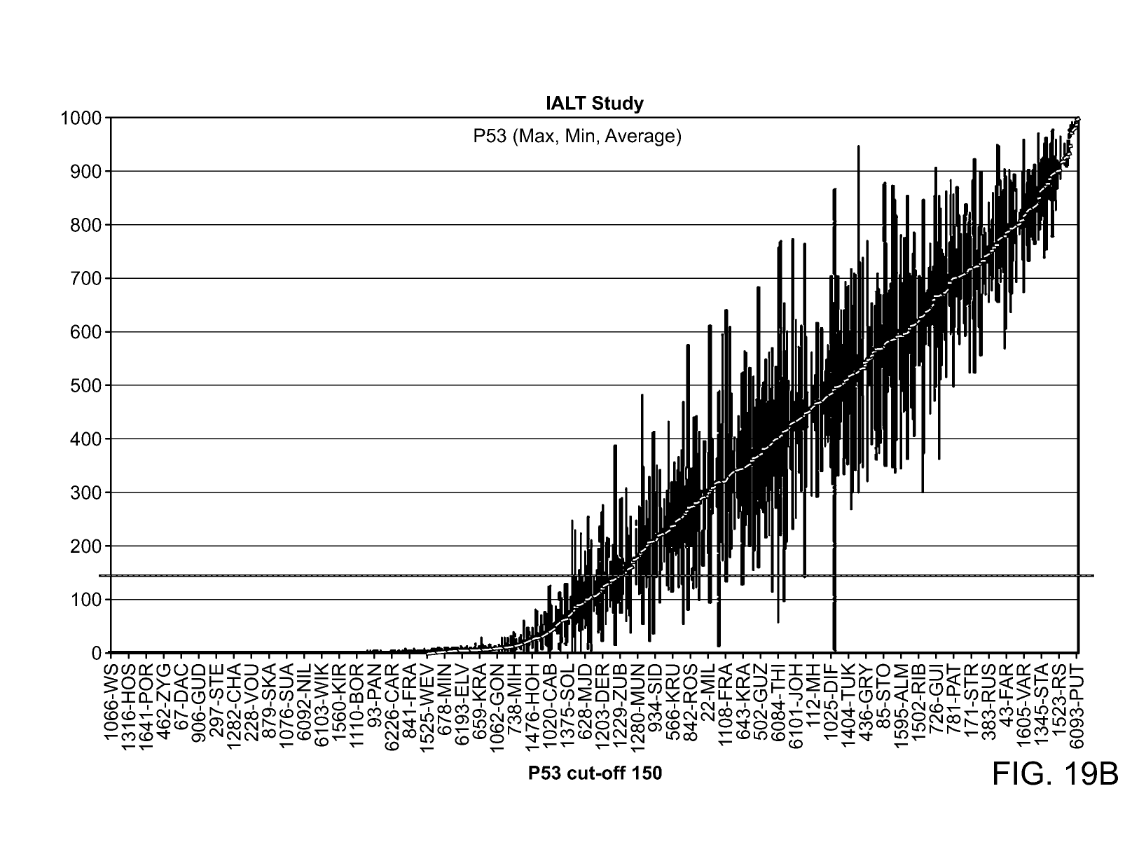

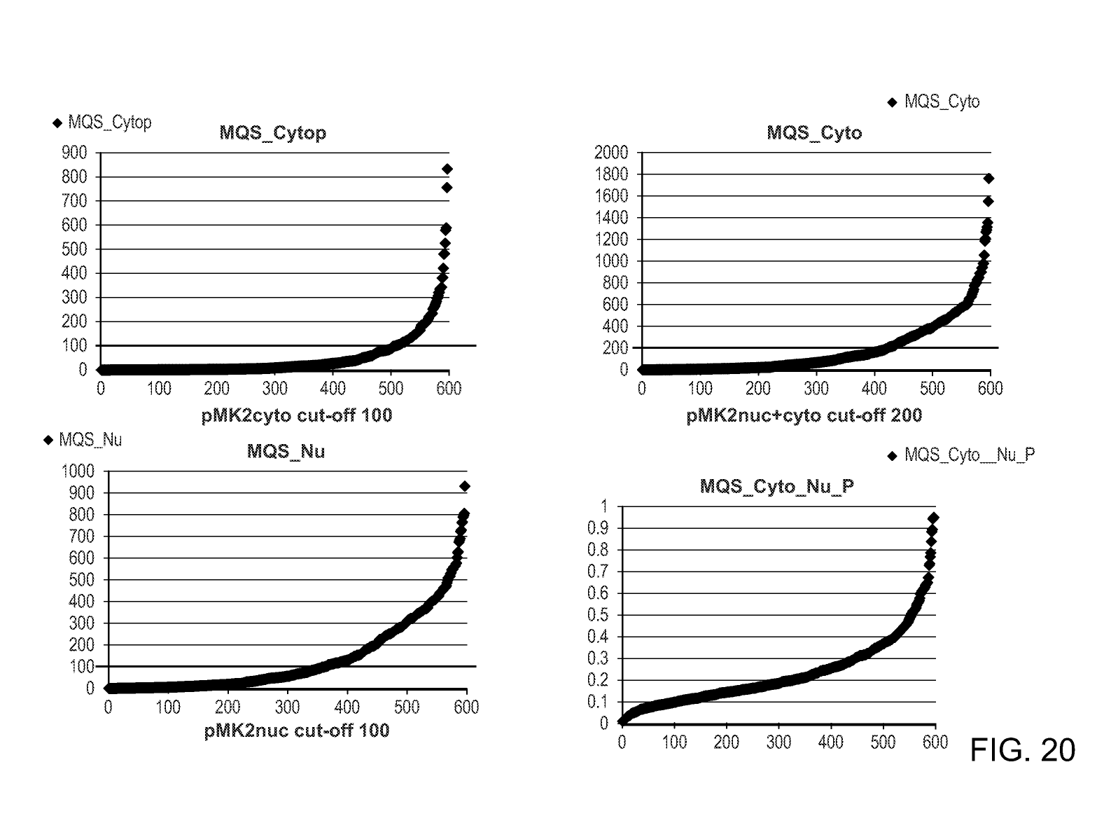

FIG. 11 further illustrates how tumor heterogeneity can affect the overall QIM score. Here, 3 different tumor regions from the same patient were scored for staining intensity. One can see that the staining is distinct within and across each of the 3 sample regions and how the overall average QIM can be affected by individual constituent QIMs derived from each scored area.

### DEFINITIONS

“Accuracy” refers to the degree of conformity of a measured or calculated quantity (a test reported value) to its actual (or true) value. Clinical accuracy relates to the proportion of true outcomes (true positives (TP) or true negatives (TN) versus misclassified outcomes (false positives (FP) or false negatives (FN)), and may be stated as a sensitivity, specificity, positive predictive values (PPV) or negative predictive values (NPV), or as a likelihood, odds ratio, among other measures.

“Biomarker” in the context of the present invention encompasses, without limitation, proteins, nucleic acids, and metabolites, together with their polymorphisms, mutations, variants, modifications, subunits, fragments, protein-ligand complexes, and degradation products, protein-ligand complexes, elements, related metabolites, and other analytes or sample-derived measures. Biomarkers can also include mutated proteins or mutated nucleic acids. Biomarkers also encompass non-blood borne factors or non-analyte physiological markers of health status, such as “clinical parameters” defined herein, as well as “traditional laboratory risk factors”, also defined herein. Biomarkers also include any calculated indices created mathematically or combinations of any one or more of the foregoing measurements, including temporal trends and differences. Where available, and unless otherwise described herein, determinants which are gene products are identified based on the official letter abbreviation or gene symbol assigned by the international Human Genome Organization Naming Committee (HGNC) and listed at the date of this filing at the US National Center for Biotechnology Information (NCBI) web site

“Circulating endothelial cell” (“CEC”) is an endothelial cell from the inner wall of blood vessels which sheds into the bloodstream under certain circumstances, including inflammation, and contributes to the formation of new vasculature associated with cancer pathogenesis. CECs may be useful as a marker of tumor progression and/or response to antiangiogenic therapy.

“Circulating tumor cell” (“CTC”) is a tumor cell of epithelial origin which is shed from the primary tumor upon metastasis, and enters the circulation. The number of circulating tumor cells in peripheral blood is associated with prognosis in patients with metastatic cancer. These cells can be separated and quantified using immunologic methods that detect epithelial cells.

“Clinical indicator” is any physiological datum used alone or in conjunction with other data in evaluating the physiological condition of a collection of cells or of an organism. This term includes pre-clinical indicators. Clinical indicators include disease free survival time, or overall survival.

“Clinical parameters” encompasses all non-sample or non-analyte biomarkers of subject health status or other characteristics, such as, without limitation, age (Age), ethnicity (RACE), gender (Sex), or family history (FamHX).

“FN” is false negative, which for a disease state test means classifying a disease subject incorrectly as non-disease or normal.

“FP” is false positive, which for a disease state test means classifying a normal subject incorrectly as having disease.

A “formula,” “algorithm,” or “model” is any mathematical equation, algorithmic, analytical or programmed process, or statistical technique that takes one or more continuous or categorical inputs (herein called “parameters”) and calculates an output value, sometimes referred to as an “index” or “index value.” Non-limiting examples of “formulas” include sums, ratios, and regression operators, such as coefficients or exponents, biomarker value transformations and normalizations (including, without limitation, those normalization schemes based on clinical parameters, such as gender, age, or ethnicity), rules and guidelines, statistical classification models, and neural networks trained on historical populations. Of particular use in combining DNARMARKERS and other biomarkers are linear and non-linear equations and statistical classification analyses to determine the relationship between levels of DNARMARKERS detected in a subject sample and the subject's responsivenss to chemotherapy. In panel and combination construction, of particular interest are structural and synactic statistical classification algorithms, and methods of risk index construction, utilizing pattern recognition features, including established techniques such as cross-correlation, Principal Components Analysis (PCA), factor rotation, Logistic Regression (LogReg), Linear Discriminant Analysis (LDA), Eigengene Linear Discriminant Analysis (ELDA), Support Vector Machines (SVM), Random Forest (RF), Recursive Partitioning Tree (RPART), as well as other related decision tree classification techniques, Shrunken Centroids (SC), StepAIC, Kth-Nearest Neighbor, Boosting, Decision Trees, Neural Networks, Bayesian Networks, Support Vector Machines, and Hidden Markov Models, among others. Other techniques may be used in survival and time to event hazard analysis, including Cox, Weibull, Kaplan-Meier and Greenwood models well known to those of skill in the art. Many of these techniques are useful either combined with a DNARMARKER selection technique, such as forward selection, backwards selection, or stepwise selection, complete enumeration of all potential panels of a given size, genetic algorithms, or they may themselves include biomarker selection methodologies in their own technique. These may be coupled with information criteria, such as Akaike's Information Criterion (AIC) or Bayes Information Criterion (BIC), in order to quantify the tradeoff between additional biomarkers and model improvement, and to aid in minimizing overfit. The resulting predictive models may be validated in other studies, or cross-validated in the study they were originally trained in, using such techniques as Bootstrap, Leave-One-Out (LOO) and 10-Fold cross-validation (10-Fold CV). At various steps, false discovery rates may be estimated by value permutation according to techniques known in the art. A “health economic utility function” is a formula that is derived from a combination of the expected probability of a range of clinical outcomes in an idealized applicable patient population, both before and after the introduction of a diagnostic or therapeutic intervention into the standard of care. It encompasses estimates of the accuracy, effectiveness and performance characteristics of such intervention, and a cost and/or value measurement (a utility) associated with each outcome, which may be derived from actual health system costs of care (services, supplies, devices and drugs, etc.) and/or as an estimated acceptable value per quality adjusted life year (QALY) resulting in each outcome. The sum, across all predicted outcomes, of the product of the predicted population size for an outcome multiplied by the respective outcomes expected utility is the total health economic utility of a given standard of care. The difference between (i) the total health economic utility calculated for the standard of care with the intervention versus (ii) the total health economic utility for the standard of care without the intervention results in an overall measure of the health economic cost or value of the intervention. This may itself be divided amongst the entire patient group being analyzed (or solely amongst the intervention group) to arrive at a cost per unit intervention, and to guide such decisions as market positioning, pricing, and assumptions of health system acceptance. Such health economic utility functions are commonly used to compare the cost-effectiveness of the intervention, but may also be transformed to estimate the acceptable value per QALY the health care system is willing to pay, or the acceptable cost-effective clinical performance characteristics required of a new intervention.

For diagnostic (or prognostic) interventions of the invention, as each outcome (which in a disease classifying diagnostic test may be a TP, FP, TN, or FN) bears a different cost, a health economic utility function may preferentially favor sensitivity over specificity, or PPV over NPV based on the clinical situation and individual outcome costs and value, and thus provides another measure of health economic performance and value which may be different from more direct clinical or analytical performance measures. These different measurements and relative trade-offs generally will converge only in the case of a perfect test, with zero error rate (a.k.a., zero predicted subject outcome misclassifications or FP and FN), which all performance measures will favor over imperfection, but to differing degrees.

“Measuring” or “measurement,” or alternatively “detecting” or “detection,” means assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's non-analyte clinical parameters.

“Negative predictive value” or “NPV” is calculated by TN/(TN+FN) or the true negative fraction of all negative test results. It also is inherently impacted by the prevalence of the disease and pre-test probability of the population intended to be tested.

See, e.g., O'Marcaigh A S, Jacobson R M, “Estimating The Predictive Value Of A Diagnostic Test, How To Prevent Misleading Or Confusing Results,” Clin. Ped. 1993, 32(8): 485-491, which discusses specificity, sensitivity, and positive and negative predictive values of a test, e.g., a clinical diagnostic test. Often, for binary disease state classification approaches using a continuous diagnostic test measurement, the sensitivity and specificity is summarized by Receiver Operating Characteristics (ROC) curves according to Pepe et al, “Limitations of the Odds Ratio in Gauging the Performance of a Diagnostic, Prognostic, or Screening Marker,” Am. J. Epidemiol 2004, 159 (9): 882-890, and summarized by the Area Under the Curve (AUC) or c-statistic, an indicator that allows representation of the sensitivity and specificity of a test, assay, or method over the entire range of test (or assay) cut points with just a single value. See also, e.g., Shultz, “Clinical Interpretation Of Laboratory Procedures,” chapter 14 in Teitz, Fundamentals of Clinical Chemistry, Burns and Ashwood (eds.), 4^{th} edition 1996, W.B. Saunders Company, pages 192-199; and Zweig et al., “ROC Curve Analysis An Example Showing The Relationships Among Serum Lipid And Apolipoprotein Concentrations In Identifying Subjects With Coronory Artery Disease,” Clin. Chem., 1992, 38(8): 1425-1428. An alternative approach using likelihood functions, odds ratios, information theory, predictive values, calibration (including goodness-of-fit), and reclassification measurements is summarized according to Cook, “Use and Misuse of the Receiver Operating Characteristic Curve in Risk Prediction,” Circulation 2007, 115: 928-935.

Finally, hazard ratios and absolute and relative risk ratios within subject cohorts defined by a test are a further measurement of clinical accuracy and utility. Multiple methods are frequently used to defining abnormal or disease values, including reference limits, discrimination limits, and risk thresholds.

“Outcome category”, synonymous with “outcome” refers to a particular category of a “categorical outcome variable”

Outcome score”, synonymous with “outcome value”, refers to a quantitative value associated with a given category or level of an ‘outcome variable’.

“Outcome variable” is a variable containing at least one set of scores that are believed to be correlated with an underlying biological condition of the cases, and may be categorical (“categorical outcome variable”) which may be nominal or ordinal, continuous or may denote an event history.

“Non-small cell lung cancer” is a group of lung cancers that are named for the kinds of cells found in the cancer and how the cells look under a microscope. The three main types of non-small cell lung cancer are squamous cell carcinoma, large cell carcinoma, and adenocarcinoma. Non-small cell lung cancer is the most common kind of lung cancer.

“Analytical accuracy” refers to the reproducibility and predictability of the measurement process itself, and may be summarized in such measurements as coefficients of variation, and tests of concordance and calibration of the same samples or controls with different times, users, equipment and/or reagents. These and other considerations in evaluating new biomarkers are also summarized in Vasan, 2006.

“Performance” is a term that relates to the overall usefulness and quality of a diagnostic or prognostic test, including, among others, clinical and analytical accuracy, other analytical and process characteristics, such as use characteristics (e.g., stability, ease of use), health economic value, and relative costs of components of the test. Any of these factors may be the source of superior performance and thus usefulness of the test, and may be measured by appropriate “performance metrics,” such as AUC, time to result, shelf life, etc. as relevant.

“Positive predictive value” or “PPV” is calculated by TP/(TP+FP) or the true positive fraction of all positive test results. It is inherently impacted by the prevalence of the disease and pre-test probability of the population intended to be tested.

“Risk” in the context of the present invention, relates to the probability that an event will occur over a specific time period, as in the responsiveness to treatment, and can can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion.

“Risk evaluation,” or “evaluation of risk” in the context of the present invention encompasses making a prediction of the probability, odds, or likelihood that an event or disease state may occur, the rate of occurrence of the event or conversion from one disease state. Risk evaluation can also comprise prediction of future clinical parameters, traditional laboratory risk factor values, or other indices of cancer, either in absolute or relative terms in reference to a previously measured population. The methods of the present invention may be used to make continuous or categorical measurements of the responsiveness to treatment thus diagnosing and defining the risk spectrum of a category of subjects defined as being at responders or non-responders. In the categorical scenario, the invention can be used to discriminate between normal and other subject cohorts at higher risk for responding. Such differing use may require different DNARMARKER combinations and individualized panels, mathematical algorithms, and/or cut-off points, but be subject to the same aforementioned measurements of accuracy and performance for the respective intended use.

A “sample” in the context of the present invention is a biological sample isolated from a subject and can include, by way of example and not limitation, tissue biopsies, whole blood, serum, plasma, blood cells, endothelial cells, lymphatic fluid, ascites fluid, interstitital fluid (also known as “extracellular fluid” and encompasses the fluid found in spaces between cells, including, inter alia, gingival crevicular fluid), bone marrow, cerebrospinal fluid (CSF), saliva, mucous, sputum, sweat, urine, or any other secretion, excretion, or other bodily fluids.

“Sensitivity” is calculated by TP/(TP+FN) or the true positive fraction of disease subjects.

“Specificity” is calculated by TN/(TN+FP) or the true negative fraction of non-disease or normal subjects.

By “statistically significant”, it is meant that the alteration is greater than what might be expected to happen by chance alone (which could be a “false positive”). Statistical significance can be determined by any method known in the art. Commonly used measures of significance include the p-value, which presents the probability of obtaining a result at least as extreme as a given data point, assuming the data point was the result of chance alone. A result is often considered highly significant at a p-value of 0.05 or less.

A “subject” in the context of the present invention is preferably a mammal. The mammal can be a human, non-human primate, mouse, rat, dog, cat, horse, or cow, but are not limited to these examples. Mammals other than humans can be advantageously used as subjects that represent animal models of cancer. A subject can be male or female.

“Survivability” refers to the ability to remain alive or continue to exist (i.e., alive or dead).

“Survival time” refers to the length or period of time a subject is able to remain alive or continue to exist as measured from an initial date (e.g., date of birth, date of diagnosis of a particular disease or stage of disease, date of initiating a therapeutic regimen, etc.) to a later date in time (e.g., date of death, date of termination of a particular therapeutic regimen, or an arbitrary date).

“Therapy” or “therapeutic regimen” includes all interventions whether biological, chemical, physical, metaphysical, or combination of the foregoing, intended to sustain or alter the monitored biological condition of a subject.

“TN” is true negative, which for a disease state test means classifying a non-disease or normal subject correctly.

“TP” is true positive, which for a disease state test means correctly classifying a disease subject.

DNA Repair and DNA Damage Response Markers

Patients have varying degrees of responsiveness to therapy and methods are needed to distinguish the capability of the treatment in a dynamic manner. Identification of changes (e.g., active, hyperactive, repressed, downmodulated, or inactive) to the cellular DNA repair pathways are useful in monitoring and predicting the response to a therapeutic compound. Accordingly, included in the invention are biomarkers associated with DNA repair and DNA damage response. The invention features methods for identifying subjects who either are or are pre-disposed to developing resistance or are sensitive to a therapeutic compound, e.g., a chemotherapeutic drug by detection of the biomarkers disclosed herein. These biomarkers are also useful for monitoring subjects undergoing treatments and therapies for cancer and cell proliferative disorders, and for selecting therapies and treatments that would be efficacious in subjects having cancer and cell proliferative disorders.

The term “biomarker” in the context of the present invention encompasses, without limitation, proteins, nucleic acids, polymorphisms of proteins and nucleic acids, elements, metabolites, and other analytes. Biomarkers can also include mutated proteins or mutated nucleic acids. The term “analyte” as used herein can mean any substance to be measured and can encompass electrolytes and elements, such as calcium.

Proteins, nucleic acids, polymorphisms, and metabolites whose levels are changed in subjects who have resistance or sensitivity to therapeutic compound, or are predisposed to developing resistance or sensitivity to therapeutic compound are summarized in Table 1 and are collectively referred to herein as, inter alia, “DNA Repair and DNA damage response proteins or DNARMARKER”.

Expression of the DNARMARKERS is determined at the protein or nucleic acid level using any method known in the art. For example, at the nucleic acid level Northern hybridization analysis using probes which specifically recognize one or more of these sequences can be used to determine gene expression. Alternatively, expression is measured using reverse-transcription-based PCR assays, e.g., using primers specific for the differentially expressed sequence of genes. Expression is also determined at the protein level, i.e., by measuring the levels of peptides encoded by the gene products described herein, or activities thereof. Such methods are well known in the art and include, e.g., immunoassays based on antibodies to proteins encoded by the genes, aptamers or molecular imprints. Any biological material can be used for the detection/quantification of the protein or its activity. Alternatively, a suitable method can be selected to determine the activity of proteins encoded by the marker genes according to the activity of each protein analyzed.

The DNARMARKER proteins are detected in any suitable manner, but are typically detected by contacting a sample from the patient with an antibody which binds the DNARMARKER protein and then detecting the presence or absence of a reaction product. The antibody may be monoclonal, polyclonal, chimeric, or a fragment of the foregoing, as discussed in detail above, and the step of detecting the reaction product may be carried out with any suitable immunoassay. The sample from the subject is typically a biological fluid as described above, and may be the same sample of biological fluid used to conduct the method described above. The sample may also be in the form of a tissue specimen from a patient where the specimen is suitable for immunohistochemistry in a variety of formats such as paraffin-embedded tissue, frozen sections of tissue, and freshly isolated tissue. The immunodetection methods are antibody-based but there are numerous additional techniques that allow for highly sensitive determinations of binding to an antibody in the context of a tissue. Those skilled in the art will be familiar with various immunohistochemistry strategies.

Immunoassays carried out in accordance with the present invention may be homogeneous assays or heterogeneous assays. In a homogeneous assay the immunological reaction usually involves the specific antibody (e.g., anti-DNARMARKER protein antibody), a labeled analyte, and the sample of interest. The signal arising from the label is modified, directly or indirectly, upon the binding of the antibody to the labeled analyte. Both the immunological reaction and detection of the extent thereof are carried out in a homogeneous solution. Immunochemical labels which may be employed include free radicals, radioisotopes, fluorescent dyes, enzymes, bacteriophages, or coenzymes.

In a heterogeneous assay approach, the reagents are usually the sample, the antibody, and means for producing a detectable signal. Samples as described above may be used. The antibody is generally immobilized on a support, such as a bead, plate or slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the sample. Means for producing a detectable signal include the use of radioactive labels, fluorescent labels, or enzyme labels. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays are radioimmunoassays, immunofluorescence methods, chemilumenescence methods, electrochemilumenescence or enzyme-linked immunoassays.

Those skilled in the art will be familiar with numerous specific immunoassay formats and variations thereof which may be useful for carrying out the method disclosed herein. See generally E. Maggio, Enzyme-Immunoassay, (1980) (CRC Press, Inc., Boca Raton, Fla.); see also U.S. Pat. No. 4,727,022 to Skold et al. titled “Methods for Modulating Ligand-Receptor Interactions and their Application,” U.S. Pat. No. 4,659,678 to Forrest et al. titled “Immunoassay of Antigens,” U.S. Pat. No. 4,376,110 to David et al., titled “Immunometric Assays Using Monoclonal Antibodies,” U.S. Pat. No. 4,275,149 to Litman et al., titled “Macromolecular Environment Control in Specific Receptor Assays,” U.S. Pat. No. 4,233,402 to Maggio et al., titled “Reagents and Method Employing Channeling,” and U.S. Pat. No. 4,230,767 to Boguslaski et al., titled “Heterogenous Specific Binding Assay Employing a Coenzyme as Label.”

Antibodies are conjugated to a solid support suitable for a diagnostic assay (e.g., beads, plates, slides or wells formed from materials such as latex or polystyrene) in accordance with known techniques, such as passive binding. Antibodies as described herein may likewise be conjugated to detectable groups such as radiolabels (e.g., 35 S, 125 I, 131 I), enzyme labels (e.g., horseradish peroxidase, alkaline phosphatase), and fluorescent labels (e.g., fluorescein) in accordance with known techniques.

The skilled artisan can routinely make antibodies, nucleic acid probes, e.g., oligonucleotides, aptamers, siRNAs against any of the DNARMARKERS in Table 1.

The invention also includes a DNARMARKER-detection reagent, e.g., nucleic acids that specifically identify one or more DNARMARKER nucleic acids by having homologous nucleic acid sequences, such as oligonucleotide sequences, complementary to a portion of the DNARMARKER nucleic acids or antibodies to proteins encoded by the DNARMARKER nucleic acids packaged together in the form of a kit. The oligonucleotides are fragments of the DNARMARKER genes. For example the oligonucleotides are 200, 150, 100, 50, 25, 10 or less nucleotides in length. The kit may contain in separate containers a nucleic acid or antibody (either already bound to a solid matrix or packaged separately with reagents for binding them to the matrix), control formulations (positive and/or negative), and/or a detectable label. Instructions (e.g., written, tape, VCR, CD-ROM, etc.) for carrying out the assay may be included in the kit. The assay may for example be in the form of a Northern hybridization or a sandwich ELISA as known in the art.

For example, DNARMARKER detection reagent, is immobilized on a solid matrix such as a porous strip to form at least one DNARMARKER detection site. The measurement or detection region of the porous strip may include a plurality of sites containing a nucleic acid. A test strip may also contain sites for negative and/or positive controls. Alternatively, control sites are located on a separate strip from the test strip. Optionally, the different detection sites may contain different amounts of immobilized nucleic acids, i.e., a higher amount in the first detection site and lesser amounts in subsequent sites. Upon the addition of test sample, the number of sites displaying a detectable signal provides a quantitative indication of the amount of DNARMARKER present in the sample. The detection sites may be configured in any suitably detectable shape and are typically in the shape of a bar or dot spanning the width of a test strip.

Alternatively, the kit contains a nucleic acid substrate array comprising one or more nucleic acid sequences. The nucleic acids on the array specifically identify one or more nucleic acid sequences represented by DNARMARKER 1-259. In various embodiments, the expression of 2, 3, 4, 5, 6, 7, 8, 9, 10, 15, 20, 25, 40 or 50 or more of the sequences represented by DNARMARKER 1-259 are identified by virtue of binding to the array. The substrate array can be on, e.g., a solid substrate, e.g., a “chip” as described in U.S. Pat. No. 5,744,305. Alternatively the substrate array can be a solution array, e.g., Luminex, Cyvera, Vitra and Quantum Dots' Mosaic.

Preferably, the kit contains antibodies for the detection of DNARMARKERS

Therapeutic Methods

Responsiveness (e.g., resistance or sensitivity) of a cell to an agent is determined by measuring an effective amount of a DNARMARKER proteins, nucleic acids, polymorphisms, metabolites, and other analytes (which may be two or more) in a test sample (e.g., a subject derived sample), and comparing the effective amounts to reference or index values, often utilizing mathematical algorithms or formula in order to combine information from results of multiple individual DNARMARKERS and from non-analyte clinical parameters into a single measurement or index. The cell is for example a cancer cell. Optionally, the cancer is a non-small cell lung cancer. The DNARMARKER is for example, XPF, FANCD2, pMK2, PAR, MLH1, PARP1, pH2AX, pHSP27, BRCA1, BRCA2, RAD51, NQO1, p53, ERCC1, ATM, or MSH2. In some embodiments the DNARMARKER is for example, MSH2, ATM and/or ATM. In another embodiment the DNARMARKER is for example pMK2, p53, ERCC′, ATM and or PARP1. In yet another embodiment the DNARMARKER is MSH2, pMK2, ATM and/or ATM.

By resistance is meant that the failure of a cell to respond to an agent. For example, resistance to a chemotherapeutic drug means the cell is not damaged or killed by the drug. By sensitivity is meant that that the cell responds to an agent. For example, sensitivity to a chemotherapeutic drug means the cell is damaged or killed by the drug. Chemotherapy includes platinum based therapy such as cisplatin.

For example, responsiveness of a cell to a chemotherapeutic agent identified by identifying a decrease in expression or activity one or more DNARMARKERS. The presence of a deficiency in DNARMARKER indicates that the cell is sensitive to a chemotherapeutic agent. Whereas, the absence of a deficiency indicates that the cell is resistant to a chemotherapeutic agent.

The methods are useful to treat, alleviate the symptoms of, diagnose, prognose monitor the progression, predict the progression of or delay the onset of cancer in a subject.

Expression of an effective amount of DNARMARKER proteins, nucleic acids or metabolites also allows for the course of treatment of cancer or a cell proliferative disorder to be monitored. In this method, a biological sample is provided from a subject undergoing treatment, e.g., chemotherapeutic treatment, for cancer or a cell proliferative disorder. If desired, biological samples are obtained from the subject at various time points before, during, or after treatment. Expression of an effective amount of DNARMARKER proteins, nucleic acids or metabolites is then determined and compared to a reference, e.g. a control individual or population whose cancer or a cell proliferative disorder state is known or an index value. The reference sample or index value may be taken or derived from one or more individuals who have been exposed to the treatment. Alternatively, the reference sample or index value may be taken or derived from one or more individuals who have not been exposed to the treatment. For example, samples may be collected from subjects who have received initial treatment for cancer or a cell proliferative disorder and subsequent treatment for diabetes to monitor the progress of the treatment.

The amount of the DNARMARKER protein, nucleic acid, polymorphism, metabolite, or other analyte can be measured in a test sample and compared to the “normal control level,” utilizing techniques such as reference limits, discrimination limits, or risk defining thresholds to define cutoff points and abnormal values. Such normal control level and cutoff points may vary based on whether a DNARMARKER is used alone or in a formula combining with other DNARMARKERS into an index. Alternatively, the normal control level can be a database of DNARMARKER patterns from previously tested subjects who responded to chemotherapy over a clinically relevant time horizon.

The present invention may be used to make continuous or categorical measurements of the response to chemotherapy or cancer survival, thus diagnosing and defining the risk spectrum of a category of subjects defined as at risk for not responding to chemotherapy. In the categorical scenario, the methods of the present invention can be used to discriminate between treatment responsive and treatment non-responsive subject cohorts. In other embodiments, the present invention may be used so as to discriminate those who have an improved survival potential. Such differing use may require different DNARMARKER combinations in individual panel, mathematical algorithm, and/or cut-off points, but be subject to the same aforementioned measurements of accuracy and other performance metrics relevant for the intended use.

Identifying the subject who will be responsive to therapy enables the selection and initiation of various therapeutic interventions or treatment regimens in order increase the individual's survival potential. Levels of an effective amount of DNARMARKER proteins, nucleic acids, polymorphisms, metabolites, or other analytes also allows for the course of treatment of a metastatic disease or metastatic event to be monitored. In this method, a biological sample can be provided from a subject undergoing treatment regimens, e.g., drug treatments, for cancer. If desired, biological samples are obtained from the subject at various time points before, during, or after treatment.

Levels of an effective amount of DNARMARKER proteins, nucleic acids, polymorphisms, metabolites, or other analytes can then be determined and compared to a reference value, e.g. a control subject or population whose therapeutic responsiveness is known or an index value or baseline value. The reference sample or index value or baseline value may be taken or derived from one or more subjects who have been exposed to the treatment, or may be taken or derived from one or more subjects who are at low risk of surviving the cancer, or may be taken or derived from subjects who have shown improvements in as a result of exposure to treatment. Alternatively, the reference sample or index value or baseline value may be taken or derived from one or more subjects who have not been exposed to the treatment. For example, samples may be collected from subjects who have received initial treatment for cancer or and subsequent treatment for cancer or a metastatic event to monitor the progress of the treatment. A reference value can also comprise a value derived from risk prediction algorithms or computed indices from population studies such as those disclosed herein.

The DNARMARKERS of the present invention can thus be used to generate a “reference DNARMARKER profile” of those subjects who would or would not be expected respond to cancer treatment. The DNARMARKERS disclosed herein can also be used to generate a “subject DNARMARKER profile” taken from subjects who are responsive cancer treatment. The subject DNARMARKER profiles can be compared to a reference DNARMARKER profile to diagnose or identify subjects at risk for developing resistance to chemotherapy, to monitor the progression of disease, as well as the rate of progression of disease, and to monitor the effectiveness of treatment modalities. The reference and subject DNARMARKER profiles of the present invention can be contained in a machine-readable medium, such as but not limited to, analog tapes like those readable by a VCR, CD-ROM, DVD-ROM, USB flash media, among others. Such machine-readable media can also contain additional test results, such as, without limitation, measurements of clinical parameters and traditional laboratory risk factors. Alternatively or additionally, the machine-readable media can also comprise subject information such as medical history and any relevant family history. The machine-readable media can also contain information relating to other disease-risk algorithms and computed indices such as those described herein.

Differences in the genetic makeup of subjects can result in differences in their relative abilities to metabolize various drugs, which may modulate the symptoms or risk factors of cancer or metastatic events. Subjects that have cancer, or at risk for developing cancer or a metastatic event can vary in age, ethnicity, and other parameters. Accordingly, use of the DNARMARKERS disclosed herein, both alone and together in combination with known genetic factors for drug metabolism, allow for a pre-determined level of predictability that a putative therapeutic or prophylactic to be tested in a selected subject will be suitable for treating or preventing cancer in the subject.

To identify the therapeutic that is appropriate for a specific subject, analysis is conducted on the expression of one or more of DNARMARKER proteins, nucleic acids or metabolites is in a test sample form the subject is determined.

The pattern of DNARMARKER expression in the test sample is measured and compared to a reference profile, e.g., a therapeutic compound reference expression profile. Comparison can be performed on test and reference samples measured concurrently or at temporally distinct times. An example of the latter is the use of compiled expression information, e.g., a sequence database, which assembles information about expression levels of DNARMARKERS.

If the reference sample, e.g., a control sample is from cells that are sensitive to a therapeutic compound then a similarity in the amount of the DNARMARKER proteins in the test sample and the reference sample indicates that treatment with that therapeutic compound will be efficacious. However, a change in the amount of the DNARMARKER in the test sample and the reference sample indicates treatment with that compound will result in a less favorable clinical outcome or prognosis. In contrast, if the reference sample, e.g., a control sample is from cells that are resistant to a therapeutic compound then a similarity in the amount of the DNARMARKER proteins in the test sample and the reference sample indicates that the treatment with that compound will result in a less favorable clinical outcome or prognosis. However, a change in the amount of the DNARMARKER in the test sample and the reference sample indicates that treatment with that therapeutic compound will be efficacious.

By “efficacious” is meant that the treatment leads to a decrease in the amount of a DNARMARKER protein, or a decrease in size, prevalence, or metastatic potential of cancer in a subject. When treatment is applied prophylactically, “efficacious” means that the treatment retards or prevents cancer or a cell proliferative disorder from forming. Assessment of cancer and cell proliferative disorders is made using standard clinical protocols.

Cancer includes non-small cell lung cancers such as adenocarcinoma/bronchoalveolar, squamous cell carcinoma, or large-cell carcinoma.

The subject is preferably a mammal. The mammal is, e.g., a human, non-human primate, mouse, rat, dog, cat, horse, or cow. The subject has been previously diagnosed as having cancer or a cell proliferative disorder, and possibly has already undergone treatment for the cancer or a cell proliferative disorder.

The subject is suffering from or at risk of developing non-small cell lung cancer. Subjects suffering from or at risk of developing non-small cell lung cancer are identified by methods known in the art.

Alternatively, the deficiency is determined by measuring the expression (e.g. increase or decrease relative to a control), detecting a sequence variation or posttranslational modification of one or more DNARMARKERS described herein.

Posttranslational modification includes for example, phosphorylation, ubiquitination, sumo-ylation, acetylation, alkylation, methylation, glycylation, glycosylation, isoprenylation, lipoylation, phosphopantetheinylation, sulfation, selenation and C-terminal amidation. For example, a deficiency in the Homologous Recombination/FA pathway is determined by detecting the monoubiquitination of FANCD2. Similarly, responsiveness of cancer cell to a MAP2KAP2 inhibitor is determined by detecting phosphorylation of a MAP2KAP2 protein. Phosphorylation indicates the cell is sensitive to a MAP2KAP2 inhibitor. In contrast the absence of phosphorylation indicates the cell is resistant to a MAP2KAP2 inhibitor.

Sequence variations such as mutations and polymorphisms may include a deletion, insertion or substitution of one or more nucleotides, relative to the wild-type nucleotide sequence. The one or more variations may be in a coding or non-coding region of the nucleic acid sequence and, may reduce or abolish the expression or function of the DNA repair pathway component polypeptide. In other words, the variant nucleic acid may encode a variant polypeptide which has reduced or abolished activity or may encode a wild-type polypeptide which has little or no expression within the cell, for example through the altered activity of a regulatory element. A variant nucleic acid may have one, two, three, four or more mutations or polymorphisms relative to the wild-type sequence.

The presence of one or more variations in a nucleic acid which encodes a component of a DNA repair pathway, is determined for example by detecting, in one or more cells of a test sample, the presence of an encoding nucleic acid sequence which comprises the one or more mutations or polymorphisms, or by detecting the presence of the variant component polypeptide which is encoded by the nucleic acid sequence.

Various methods are available for determining the presence or absence in a sample obtained from an individual of a particular nucleic acid sequence, for example a nucleic acid sequence which has a mutation or polymorphism that reduces or abrogates the expression or activity of a DNA repair pathway component. Furthermore, having sequenced nucleic acid of an individual or sample, the sequence information can be retained and subsequently searched without recourse to the original nucleic acid itself. Thus, for example, scanning a database of sequence information using sequence analysis software may identify a sequence alteration or mutation.

Methods according to some aspects of the present invention may comprise determining the binding of an oligonucleotide probe to nucleic acid obtained from the sample, for example, genomic DNA, RNA or cDNA. The probe may comprise a nucleotide sequence which binds specifically to a nucleic acid sequence which contains one or more mutations or polymorphisms and does not bind specifically to the nucleic acid sequence which does not contain the one or more mutations or polymorphisms, or vice versa. The oligonucleotide probe may comprise a label and binding of the probe may be determined by detecting the presence of the label.

A method may include hybridization of one or more (e.g. two) oligonucleotide probes or primers to target nucleic acid. Where the nucleic acid is double-stranded DNA, hybridization will generally be preceded by denaturation to produce single-stranded DNA. The hybridization may be as part of a PCR procedure, or as part of a probing procedure not involving PCR. An example procedure would be a combination of PCR and low stringency hybridization.

Binding of a probe to target nucleic acid (e.g. DNA) may be measured using any of a variety of techniques at the disposal of those skilled in the art. For instance, probes may be radioactively, fluorescently or enzymatically labeled. Other methods not employing labeling of probe include examination of restriction fragment length polymorphisms, amplification using PCR, RNase cleavage and allele specific oligonucleotide probing. Probing may employ the standard Southern blotting technique. For instance, DNA may be extracted from cells and digested with different restriction enzymes. Restriction fragments may then be separated by electrophoresis on an agarose gel, before denaturation and transfer to a nitrocellulose filter. Labeled probe may be hybridized to the DNA fragments on the filter and binding determined.

Those skilled in the art are well able to employ suitable conditions of the desired stringency for selective hybridization, taking into account factors such as oligonucleotide length and base composition, temperature and so on. Suitable selective hybridization conditions for oligonucleotides of 17 to 30 bases include hybridization overnight at 42° C. in 6×SSC and washing in 6×SSC at a series of increasing temperatures from 42° C. to 65° C. Other suitable conditions and protocols are described in Molecular Cloning: a Laboratory Manual: 3rd edition, Sambrook & Russell (2001) Cold Spring Harbor Laboratory Press NY and Current Protocols in Molecular Biology, Ausubel et al. eds. John Wiley & Sons (1992).

Nucleic acid, which may be genomic DNA, RNA or cDNA, or an amplified region thereof, may be sequenced to identify or determine the presence of polymorphism or mutation therein. A polymorphism or mutation may be identified by comparing the sequence obtained with the database sequence of the component, as set out above. In particular, the presence of one or more polymorphisms or mutations that cause abrogation or loss of function of the polypeptide component, and thus the DNA repair pathway as a whole, may be determined.

Sequencing may be performed using any one of a range of standard techniques. Sequencing of an amplified product may, for example, involve precipitation with isopropanol, resuspension and sequencing using a TaqFS+ Dye terminator sequencing kit. Extension products may be electrophoresed on an ABI 377 DNA sequencer and data analyzed using Sequence Navigator software.

A specific amplification reaction such as PCR using one or more pairs of primers may conveniently be employed to amplify the region of interest within the nucleic acid sequence, for example, the portion of the sequence suspected of containing mutations or polymorphisms. The amplified nucleic acid may then be sequenced as above, and/or tested in any other way to determine the presence or absence of a mutation or polymorphism which reduces or abrogates the expression or activity of the DNA repair pathway component. Suitable amplification reactions include the polymerase chain reaction (PCR) (reviewed for instance in “PCR protocols; A Guide to Methods and Applications”, Eds. Innis et al, 1990, Academic Press, New York, Mullis et al, Cold Spring Harbor Symp. Quant. Biol., 51:263, (1987), Ehrlich (ed), PCR technology, Stockton Press, NY, 1989, and Ehrlich et al, Science, 252:1643-1650, (1991)).

Mutations and polymorphisms associated with cancer may also be detected at the protein level by detecting the presence of a variant (i.e. a mutant or allelic variant) polypeptide.

A method of identifying a cancer cell in a sample from an individual as deficient in DNA repair may include contacting a sample with a specific binding member directed against a variant (e.g. a mutant) polypeptide component of the pathway, and determining binding of the specific binding member to the sample. Binding of the specific binding member to the sample may be indicative of the presence of the variant polypeptide component of the DNA repair pathway in a cell within the sample. Preferred specific binding molecules for use in aspects of the present invention include antibodies and fragments or derivatives thereof ('antibody molecules').

The reactivities of a binding member such as an antibody on normal and test samples may be determined by any appropriate means. Tagging with individual reporter molecules is one possibility. The reporter molecules may directly or indirectly generate detectable, and preferably measurable, signals. The linkage of reporter molecules may be directly or indirectly, covalently, e.g. via a peptide bond or non-covalently. Linkage via a peptide bond may be as a result of recombinant expression of a gene fusion encoding binding molecule (e.g. antibody) and reporter molecule.

Performance and Accuracy Measures of the Invention

The performance and thus absolute and relative clinical usefulness of the invention may be assessed in multiple ways as noted above. Amongst the various assessments of performance, the invention is intended to provide accuracy in clinical diagnosis and prognosis. The accuracy of a diagnostic or prognostic test, assay, or method concerns the ability of the test, assay, or method to distinguish between subjects responsive to chemotherapeutic treatment and those that are not, is based on whether the subjects have an “effective amount” or a “significant alteration” in the levels of a DNARMARKER. By “effective amount” or “significant alteration,” it is meant that the measurement of an appropriate number of DNARMARKERS (which may be one or more) is different than the predetermined cut-off point (or threshold value) for that DNARMARKER(S) and therefore indicates that the subject responsiveness to therapy for which the DNARMARKER(S) is a determinant. The difference in the level of DNARMARKER between normal and abnormal is preferably statistically significant. As noted below, and without any limitation of the invention, achieving statistical significance, and thus the preferred analytical and clinical accuracy, generally but not always requires that combinations of several DNARMARKERS be used together in panels and combined with mathematical algorithms in order to achieve a statistically significant DNARMARKER index.

In the categorical diagnosis of a disease state, changing the cut point or threshold value of a test (or assay) usually changes the sensitivity and specificity, but in a qualitatively inverse relationship. Therefore, in assessing the accuracy and usefulness of a proposed medical test, assay, or method for assessing a subject's condition, one should always take both sensitivity and specificity into account and be mindful of what the cut point is at which the sensitivity and specificity are being reported because sensitivity and specificity may vary significantly over the range of cut points. Use of statistics such as AUC, encompassing all potential cut point values, is preferred for most categorical risk measures using the invention, while for continuous risk measures, statistics of goodness-of-fit and calibration to observed results or other gold standards, are preferred.

Using such statistics, an “acceptable degree of diagnostic accuracy”, is herein defined as a test or assay (such as the test of the invention for determining the clinically significant presence of DNARMARKERS, which thereby indicates the presence of cancer and/or a risk of having a metastatic event) in which the AUC (area under the ROC curve for the test or assay) is at least 0.60, desirably at least 0.65, more desirably at least 0.70, preferably at least 0.75, more preferably at least 0.80, and most preferably at least 0.85.

By a “very high degree of diagnostic accuracy”, it is meant a test or assay in which the AUC (area under the ROC curve for the test or assay) is at least 0.80, desirably at least 0.85, more desirably at least 0.875, preferably at least 0.90, more preferably at least 0.925, and most preferably at least 0.95.

The predictive value of any test depends on the sensitivity and specificity of the test, and on the prevalence of the condition in the population being tested. This notion, based on Bayes' theorem, provides that the greater the likelihood that the condition being screened for is present in an individual or in the population (pre-test probability), the greater the validity of a positive test and the greater the likelihood that the result is a true positive. Thus, the problem with using a test in any population where there is a low likelihood of the condition being present is that a positive result has limited value (i.e., more likely to be a false positive). Similarly, in populations at very high risk, a negative test result is more likely to be a false negative.

As a result, ROC and AUC can be misleading as to the clinical utility of a test in low disease prevalence tested populations (defined as those with less than 1% rate of occurrences (incidence) per annum, or less than 10% cumulative prevalence over a specified time horizon). Alternatively, absolute risk and relative risk ratios as defined elsewhere in this disclosure can be employed to determine the degree of clinical utility. Populations of subjects to be tested can also be categorized into quartiles by the test's measurement values, where the top quartile (25% of the population) comprises the group of subjects with the highest relative risk for therapeutic unresponsiveness, and the bottom quartile comprising the group of subjects having the lowest relative risk for therapeutic unresponsiveness Generally, values derived from tests or assays having over 2.5 times the relative risk from top to bottom quartile in a low prevalence population are considered to have a “high degree of diagnostic accuracy,” and those with five to seven times the relative risk for each quartile are considered to have a “very high degree of diagnostic accuracy.” Nonetheless, values derived from tests or assays having only 1.2 to 2.5 times the relative risk for each quartile remain clinically useful are widely used as risk factors for a disease; such is the case with total cholesterol and for many inflammatory biomarkers with respect to their prediction of future events. Often such lower diagnostic accuracy tests must be combined with additional parameters in order to derive meaningful clinical thresholds for therapeutic intervention, as is done with the aforementioned global risk assessment indices.

A health economic utility function is an yet another means of measuring the performance and clinical value of a given test, consisting of weighting the potential categorical test outcomes based on actual measures of clinical and economic value for each. Health economic performance is closely related to accuracy, as a health economic utility function specifically assigns an economic value for the benefits of correct classification and the costs of misclassification of tested subjects. As a performance measure, it is not unusual to require a test to achieve a level of performance which results in an increase in health economic value per test (prior to testing costs) in excess of the target price of the test.

In general, alternative methods of determining diagnostic accuracy are commonly used for continuous measures, when a disease category or risk category has not yet been clearly defined by the relevant medical societies and practice of medicine, where thresholds for therapeutic use are not yet established, or where there is no existing gold standard for diagnosis of the pre-disease. For continuous measures of risk, measures of diagnostic accuracy for a calculated index are typically based on curve fit and calibration between the predicted continuous value and the actual observed values (or a historical index calculated value) and utilize measures such as R squared, Hosmer-Lemeshow P-value statistics and confidence intervals. It is not unusual for predicted values using such algorithms to be reported including a confidence interval (usually 90% or 95% CI) based on a historical observed cohort's predictions, as in the test for risk of future breast cancer recurrence commercialized by Genomic Health, Inc. (Redwood City, Calif.).

In general, by defining the degree of diagnostic accuracy, i.e., cut points on a ROC curve, defining an acceptable AUC value, and determining the acceptable ranges in relative concentration of what constitutes an effective amount of the DNARMARKERS of the invention allows for one of skill in the art to use the DNARMARKERS to identify, diagnose, or prognose subjects with a pre-determined level of predictability and performance.

Construction of DNARMARKER Panels

Groupings of DNARMARKERS can be included in “panels.” A “panel” within the context of the present invention means a group of biomarkers (whether they are DNARMARKERS, clinical parameters, or traditional laboratory risk factors) that includes more than one DNARMARKER. A panel can also comprise additional biomarkers, e.g., clinical parameters, traditional laboratory risk factors, known to be present or associated with responsiveness to chemotherapeutic treatment, in combination with a selected group of the DNARMARKERS listed in Table 1 or Table 2. Optionally the panel includes markers listed in Tables 3 and 4.

As noted above, many of the individual DNARMARKERS, clinical parameters, and traditional laboratory risk factors listed, when used alone and not as a member of a multi-biomarker panel of DNARMARKERS, have little or no clinical use in reliably distinguishing individuals that are responsive to therapeutic treatment and those that are not and thus cannot reliably be used alone in classifying any subject between those two states. Even where there are statistically significant differences in their mean measurements in each of these populations, as commonly occurs in studies which are sufficiently powered, such biomarkers may remain limited in their applicability to an individual subject, and contribute little to diagnostic or prognostic predictions for that subject. A common measure of statistical significance is the p-value, which indicates the probability that an observation has arisen by chance alone; preferably, such p-values are 0.05 or less, representing a 5% or less chance that the observation of interest arose by chance. Such p-values depend significantly on the power of the study performed.

Despite this individual DNARMARKER performance, and the general performance of formulas combining only the traditional clinical parameters and few traditional laboratory risk factors, the present inventors have noted that certain specific combinations of two or more DNARMARKERS can also be used as multi-biomarker panels comprising combinations of DNARMARKERS that are known to be involved in one or more physiological or biological pathways, and that such information can be combined and made clinically useful through the use of various formulae, including statistical classification algorithms and others, combining and in many cases extending the performance characteristics of the combination beyond that of the individual DNARMARKERS. These specific combinations show an acceptable level of diagnostic accuracy, and, when sufficient information from multiple DNARMARKERS is combined in a trained formula, often reliably achieve a high level of diagnostic accuracy transportable from one population to another.

The general concept of how two less specific or lower performing DNARMARKERS are combined into novel and more useful combinations for the intended indications, is a key aspect of the invention. Multiple biomarkers can often yield better performance than the individual components when proper mathematical and clinical algorithms are used; this is often evident in both sensitivity and specificity, and results in a greater AUC. Secondly, there is often novel unperceived information in the existing biomarkers, as such was necessary in order to achieve through the new formula an improved level of sensitivity or specificity. This hidden information may hold true even for biomarkers which are generally regarded to have suboptimal clinical performance on their own. In fact, the suboptimal performance in terms of high false positive rates on a single biomarker measured alone may very well be an indicator that some important additional information is contained within the biomarker results—information which would not be elucidated absent the combination with a second biomarker and a mathematical formula.

Several statistical and modeling algorithms known in the art can be used to both assist in DNARMARKER selection choices and optimize the algorithms combining these choices. Statistical tools such as factor and cross-biomarker correlation/covariance analyses allow more rationale approaches to panel construction. Mathematical clustering and classification trees showing the Euclidean standardized distance between the DNARMARKERS can be advantageously used. Pathway informed seeding of such statistical classification techniques also may be employed, as may rational approaches based on the selection of individual DNARMARKERS based on their participation across in particular pathways or physiological functions.

Ultimately, formula such as statistical classification algorithms can be directly used to both select DNARMARKERS and to generate and train the optimal formula necessary to combine the results from multiple DNARMARKERS into a single index. Often, techniques such as forward (from zero potential explanatory parameters) and backwards selection (from all available potential explanatory parameters) are used, and information criteria, such as AIC or BIC, are used to quantify the tradeoff between the performance and diagnostic accuracy of the panel and the number of DNARMARKERS used. The position of the individual DNARMARKER on a forward or backwards selected panel can be closely related to its provision of incremental information content for the algorithm, so the order of contribution is highly dependent on the other constituent DNARMARKERS in the panel.

Construction of Clinical Algorithms

Any formula may be used to combine DNARMARKER results into indices useful in the practice of the invention. As indicated above, and without limitation, such indices may indicate, among the various other indications, the probability, likelihood, absolute or relative chance of responding to chemotherapy. This may be for a specific time period or horizon, or for remaining lifetime risk, or simply be provided as an index relative to another reference subject population.

Although various preferred formula are described here, several other model and formula types beyond those mentioned herein and in the definitions above are well known to one skilled in the art. The actual model type or formula used may itself be selected from the field of potential models based on the performance and diagnostic accuracy characteristics of its results in a training population. The specifics of the formula itself may commonly be derived from DNARMARKER results in the relevant training population. Amongst other uses, such formula may be intended to map the feature space derived from one or more DNARMARKER inputs to a set of subject classes (e.g. useful in predicting class membership of subjects as normal, responders and non-responders), to derive an estimation of a probability function of risk using a Bayesian approach (e.g. the risk of cancer or a metastatic event), or to estimate the class-conditional probabilities, then use Bayes' rule to produce the class probability function as in the previous case.

Preferred formulas include the broad class of statistical classification algorithms, and in particular the use of discriminant analysis. The goal of discriminant analysis is to predict class membership from a previously identified set of features. In the case of linear discriminant analysis (LDA), the linear combination of features is identified that maximizes the separation among groups by some criteria. Features can be identified for LDA using an eigengene based approach with different thresholds (ELDA) or a stepping algorithm based on a multivariate analysis of variance (MANOVA). Forward, backward, and stepwise algorithms can be performed that minimize the probability of no separation based on the Hotelling-Lawley statistic.

Eigengene-based Linear Discriminant Analysis (ELDA) is a feature selection technique developed by Shen et al. (2006). The formula selects features (e.g. biomarkers) in a multivariate framework using a modified eigen analysis to identify features associated with the most important eigenvectors. “Important” is defined as those eigenvectors that explain the most variance in the differences among samples that are trying to be classified relative to some threshold.

A support vector machine (SVM) is a classification formula that attempts to find a hyperplane that separates two classes. This hyperplane contains support vectors, data points that are exactly the margin distance away from the hyperplane. In the likely event that no separating hyperplane exists in the current dimensions of the data, the dimensionality is expanded greatly by projecting the data into larger dimensions by taking non-linear functions of the original variables (Venables and Ripley, 2002). Although not required, filtering of features for SVM often improves prediction. Features (e.g., biomarkers) can be identified for a support vector machine using a non-parametric Kruskal-Wallis (KW) test to select the best univariate features. A random forest (RF, Breiman, 2001) or recursive partitioning (RPART, Breiman et al., 1984) can also be used separately or in combination to identify biomarker combinations that are most important. Both KW and RF require that a number of features be selected from the total. RPART creates a single classification tree using a subset of available biomarkers.

Other formula may be used in order to pre-process the results of individual DNARMARKER measurement into more valuable forms of information, prior to their presentation to the predictive formula. Most notably, normalization of biomarker results, using either common mathematical transformations such as logarithmic or logistic functions, as normal or other distribution positions, in reference to a population's mean values, etc. are all well known to those skilled in the art. Of particular interest are a set of normalizations based on Clinical Parameters such as age, gender, race, or sex, where specific formula are used solely on subjects within a class or continuously combining a Clinical Parameter as an input. In other cases, analyte-based biomarkers can be combined into calculated variables which are subsequently presented to a formula.

In addition to the individual parameter values of one subject potentially being normalized, an overall predictive formula for all subjects, or any known class of subjects, may itself be recalibrated or otherwise adjusted based on adjustment for a population's expected prevalence and mean biomarker parameter values, according to the technique outlined in D'Agostino et al, (2001) JAMA 286:180-187, or other similar normalization and recalibration techniques. Such epidemiological adjustment statistics may be captured, confirmed, improved and updated continuously through a registry of past data presented to the model, which may be machine readable or otherwise, or occasionally through the retrospective query of stored samples or reference to historical studies of such parameters and statistics. Additional examples that may be the subject of formula recalibration or other adjustments include statistics used in studies by Pepe, M. S. et al, 2004 on the limitations of odds ratios; Cook, N. R., 2007 relating to ROC curves. Finally, the numeric result of a classifier formula itself may be transformed post-processing by its reference to an actual clinical population and study results and observed endpoints, in order to calibrate to absolute risk and provide confidence intervals for varying numeric results of the classifier or risk formula. An example of this is the presentation of absolute risk, and confidence intervals for that risk, derived using an actual clinical study, chosen with reference to the output of the recurrence score formula in the Oncotype Dx product of Genomic Health, Inc. (Redwood City, Calif.). A further modification is to adjust for smaller sub-populations of the study based on the output of the classifier or risk formula and defined and selected by their Clinical Parameters, such as age or sex.

### EXAMPLES

### Example 1

### DNA Repair Biomarker Evaluation as Discriminators of Clinical Endpoint Parameters in the International Adjuvant Lung Trial (IALT)

An immunohistochemical evaluation of DNA Repair Proteins was conducted on a large international patient specimen collection from the IALT trial (International Adjuvant Lung Trial). This clinical trial originally comprised approximately 1867 non-small cell lung cancer patient specimens (adenocarcinomas and squamous cell carcinoma) and was designed to test clinical parameters for patients receiving either cisplatin-based therapy or those considered observation. One problem with testing of biomarkers in general from clinical trials is that the patient population treatments are generally a mixture of several different treatments and combinations of such and so results may be confounded by interactions of various treatments. However, the IALT trial represents pristine treatment arms of cisplatin and observation and thus is relatively free of confounding affects due to treatment variation.

International Adjuvant Lung Cancer Trial (IALT) NSCLC FFPE patient specimens constructed on TMAs (13 total) were stained by IHC for DNA repair biomarkers: ATM, MSH2, ERCC1, p53, pMK2, PARP1, BRCA1, XPF. An average of 603 patients were analyzed for each biomarker. Tumor biomarker nuclear or cytoplasmic levels were determined using digital image user defined macros. Scores were generated based on weighted intensity and quantity of stained cells. In reference to accompanying diagrams in the Powerpoint files, please note that a wide dynamic range achieved during assay development and optimization in commercially available TMAs is translationally achieved when patients comprising this study population are stained, annotated for tumor region and scored by user-defined macros.

Previous studies have utilized standard pathologist-based scoring, thus we chose to optimize the IHC-based assessment of biomarker levels using digital pathology and weighted scoring. This endeavor is undertaken so that for a given population a continuum of scores (herein referred to as Q-scores) with a broad dynamic range may be generated by user defined macros. Here, scoring of individual tumor cells comprising each patient specimen is generated and then patients assembled into a population continuum of scores against which finer cut-points may tested and established rather than crude binning methods as employed in previous studies.

Q-score=10*(% of 10+cells)+9*(% of 9+cells)+8*(% of 8+cells)+7*(% of 7+cells) . . . 1*(% of 1+cells)

Cox PH models adjusted for relevant clinical and stratification variables were used in the univariate analyses of Disease-Free Survival (DFS) and Overall Survival (OS). Initial analyses were conducted using biomarker Q scores as continuous variables against clinical endpoint metrics. The results for initial analysis without adjustment for clinical variables indicated that p53 was prognostic and predictive for cisplatin treatment relative to clinical endpoint metrics.

The univariate biomarker analyses yielded significant prognostic and predictive values using disease-free survival (DFS) as the primary endpoint and overall survival (OS) as the secondary endpoint. This exploratory data support predictive modeling of DNA repair enzyme expression levels that cosegregate with SCC but not adenocarcinoma, and include MSH2 (predictive p=0.012, HR=1.218, 95% CI=[1.044-1.42]), p53 (p=0.005, HR=1.120, 95% CI=[1.035-1.212],) and ATM (p=0.010, HR=1.212, 95% CI=[1.046-1.405]).

Partition models for pMK2, p53, ERCC1, ATM, and PARP1 were statistically significant for prediction in SCC but not adenocarcinoma. XPF and BRCA1 were not predictive or prognostic in any of the models tested for patient outcome prognosis or cisplatin-predictive response.

These data suggest that expression of DNA repair enzymes represent a distinct molecular difference between SCC and adenocarcinoma and that application of biomarker modeling may be predictive for specific tumor histological subclasses. The results of this study are shown below.

| | | | | | | |
|---|---|---|---|---|---|---|
| ParameterN | Estimate | StdErr | HazardRatio | HRLowerCL | HRUpperCL | ProbChiSq |

Disease Free Survival
MSH2
ADENOCARCINOMA
Without variables

| | | | | | | |
|---|---|---|---|---|---|---|
| Treatment | 0.30287 | 0.252 | 1.354 | 0.826 | 2.219 | 0.230 |
| MSH2 | 0.02107 | 0.100 | 1.021 | 0.840 | 1.242 | 0.833 |
| MSH2_Treatment | −0.21738 | 0.148 | 0.805 | 0.602 | 1.076 | 0.143 |

MSH2 is not prognostic and not predictive.

With selected variables

| | | | | | | |
|---|---|---|---|---|---|---|
| Treatment | −0.08628 | 0.257 | 0.917 | 0.554 | 1.519 | 0.737 |
| MSH2 | −0.06292 | 0.101 | 0.939 | 0.770 | 1.145 | 0.533 |
| MSH2_Treatment | 0.00325 | 0.147 | 1.003 | 0.752 | 1.339 | 0.982 |
| T of TNM | 0.32050 | 0.147 | 1.378 | 1.033 | 1.837 | 0.029 |
| Performance Status | 0.28018 | 0.141 | 1.323 | 1.005 | 1.743 | 0.046 |
| Sex | −0.48242 | 0.185 | 0.617 | 0.430 | 0.886 | 0.009 |
| Age | 0.29878 | 0.108 | 1.348 | 1.092 | 1.665 | 0.005 |
| N of TNM | 0.86472 | 0.110 | 2.374 | 1.915 | 2.944 | 0.000 |
| Surgery | −0.42066 | 0.195 | 0.657 | 0.448 | 0.961 | 0.031 |

MSH2 is not prognostic and not predictive.

With adjustment variables

| | | | | | | |
|---|---|---|---|---|---|---|
| Treatment | −0.05376 | 0.260 | 0.948 | 0.569 | 1.578 | 0.836 |
| MSH2 | −0.09179 | 0.099 | 0.912 | 0.751 | 1.109 | 0.356 |
| MSH2_Treatment | −0.01774 | 0.148 | 0.982 | 0.735 | 1.314 | 0.905 |

MSH2 is not prognostic and not predictive.

SQUAMOUS CELLS
Without variables

| | | | | | | |
|---|---|---|---|---|---|---|
| Treatment | −0.52613 | 0.200 | 0.591 | 0.399 | 0.874 | 0.008 |
| MSH2 | −0.11878 | 0.056 | 0.888 | 0.795 | 0.992 | 0.035 |
| MSH2_Treatment | 0.20590 | 0.080 | 1.229 | 1.051 | 1.437 | 0.010 |

MSH2 is prognostic and predictive.

With selected variables

| | | | | | | |
|---|---|---|---|---|---|---|
| Treatment | −0.49703 | 0.198 | 0.608 | 0.412 | 0.897 | 0.012 |
| MSH2 | −0.10214 | 0.055 | 0.903 | 0.810 | 1.006 | 0.064 |
| MSH2_Treatment | 0.19682 | 0.079 | 1.218 | 1.044 | 1.420 | 0.012 |
| T of TNM | 0.36389 | 0.101 | 1.439 | 1.181 | 1.754 | 0.000 |
| N of TNM | 0.41423 | 0.084 | 1.513 | 1.283 | 1.785 | 0.000 |

MSH2 is not prognostic but predictive.

With adjustment variables

| | | | | | | |
|---|---|---|---|---|---|---|
| Treatment | −0.50254 | 0.200 | 0.605 | 0.409 | 0.894 | 0.012 |
| MSH2 | −0.10522 | 0.056 | 0.900 | 0.807 | 1.004 | 0.060 |
| MSH2_Treatment | 0.18636 | 0.079 | 1.205 | 1.031 | 1.408 | 0.019 |

MSH2 is not prognostic but predictive.

P53
ADENOCARCINOMA
Without variables

| | | | | | | |
|---|---|---|---|---|---|---|
| Treatment | 0.03269 | 0.194 | 1.033 | 0.707 | 1.510 | 0.866 |
| P53 | −0.03455 | 0.046 | 0.966 | 0.882 | 1.058 | 0.457 |
| P53_Treatment | −0.02939 | 0.074 | 0.971 | 0.841 | 1.122 | 0.689 |

P53 is not prognostic and not predictive.

With selected variables

| | | | | | | |
|---|---|---|---|---|---|---|
| Treatment | −0.22298 | 0.199 | 0.800 | 0.542 | 1.182 | 0.262 |
| P53 | −0.07911 | 0.048 | 0.924 | 0.842 | 1.014 | 0.097 |
| P53_Treatment | 0.06741 | 0.075 | 1.070 | 0.923 | 1.239 | 0.369 |
| T of TNM | 0.36654 | 0.144 | 1.443 | 1.088 | 1.913 | 0.011 |
| Sex | −0.42166 | 0.179 | 0.656 | 0.462 | 0.931 | 0.018 |
| Age | 0.33733 | 0.104 | 1.401 | 1.143 | 1.718 | 0.001 |
| N of TNM | 0.83949 | 0.108 | 2.315 | 1.872 | 2.863 | 0.000 |
| Surgery | −0.43242 | 0.194 | 0.649 | 0.443 | 0.950 | 0.026 |

P53 is not prognostic and not predictive.

With adjustment variables

| | | | | | | |
|---|---|---|---|---|---|---|
| Treatment | −0.17485 | 0.202 | 0.840 | 0.565 | 1.248 | 0.388 |
| P53 | −0.06774 | 0.048 | 0.935 | 0.851 | 1.026 | 0.154 |
| P53_Treatment | 0.05080 | 0.074 | 1.052 | 0.909 | 1.217 | 0.495 |

P53 is not prognostic and not predictive.

SQUAMOUS CELLS
Without variables

| | | | | | | |
|---|---|---|---|---|---|---|
| Treatment | −0.43341 | 0.170 | 0.648 | 0.465 | 0.904 | 0.011 |
| P53 | −0.07129 | 0.029 | 0.931 | 0.880 | 0.986 | 0.014 |
| P53_Treatment | 0.10739 | 0.040 | 1.113 | 1.029 | 1.205 | 0.008 |

P53 is prognostic and predictive.

With selected variables

| | | | | | | |
|---|---|---|---|---|---|---|
| Treatment | −0.45344 | 0.171 | 0.635 | 0.454 | 0.889 | 0.008 |
| P53 | −0.06651 | 0.029 | 0.936 | 0.883 | 0.991 | 0.023 |
| P53_Treatment | 0.11311 | 0.040 | 1.120 | 1.035 | 1.212 | 0.005 |
| T of TNM | 0.34207 | 0.100 | 1.408 | 1.157 | 1.714 | 0.001 |
| Performance Status | 0.21692 | 0.099 | 1.242 | 1.022 | 1.509 | 0.029 |
| N of TNM | 0.41484 | 0.085 | 1.514 | 1.283 | 1.787 | 0.000 |

P53 is prognostic and predictive.

With adjustment variables

| | | | | | | |
|---|---|---|---|---|---|---|
| Treatment | −0.45968 | 0.174 | 0.631 | 0.449 | 0.887 | 0.008 |
| P53 | −0.07145 | 0.030 | 0.931 | 0.878 | 0.987 | 0.016 |
| P53_Treatment | 0.10805 | 0.041 | 1.114 | 1.029 | 1.207 | 0.008 |

P53 is prognostic and predictive.

PARP1
ADENOCARCINOMA
Without variables

| | | | | | | |
|---|---|---|---|---|---|---|
| Treatment | −0.12330 | 0.345 | 0.884 | 0.449 | 1.739 | 0.721 |
| PARP1 | −0.03384 | 0.057 | 0.967 | 0.864 | 1.081 | 0.554 |
| PARP1_Treatment | 0.03238 | 0.076 | 1.033 | 0.890 | 1.199 | 0.670 |

PARP1 is not prognostic and not predictive.

With selected variables

| | | | | | | |
|---|---|---|---|---|---|---|
| Treatment | −0.11927 | 0.357 | 0.888 | 0.440 | 1.788 | 0.739 |
| PARP1 | −0.03652 | 0.060 | 0.964 | 0.857 | 1.085 | 0.545 |
| PARP1_Treatment | 0.01440 | 0.079 | 1.015 | 0.868 | 1.185 | 0.856 |
| T of TNM | 0.34359 | 0.144 | 1.410 | 1.063 | 1.870 | 0.017 |
| Performance Status | 0.28350 | 0.141 | 1.328 | 1.007 | 1.751 | 0.045 |
| Sex | −0.47388 | 0.185 | 0.623 | 0.433 | 0.894 | 0.010 |
| Age | 0.28797 | 0.107 | 1.334 | 1.082 | 1.644 | 0.007 |
| N of TNM | 0.86730 | 0.110 | 2.380 | 1.919 | 2.953 | 0.000 |
| Surgery | −0.41201 | 0.196 | 0.662 | 0.451 | 0.973 | 0.036 |

PARP1 is not prognostic and not predictive.

With adjustment variables

| | | | | | | |
|---|---|---|---|---|---|---|
| Treatment | −0.00601 | 0.366 | 0.994 | 0.485 | 2.036 | 0.987 |
| PARP1 | −0.04591 | 0.061 | 0.955 | 0.847 | 1.077 | 0.454 |
| PARP1_Treatment | −0.00769 | 0.080 | 0.992 | 0.848 | 1.162 | 0.924 |

PARP1 is not prognostic and not predictive.

SQUAMOUS CELLS
Without variables

| | | | | | | |
|---|---|---|---|---|---|---|
| Treatment | −0.56949 | 0.303 | 0.566 | 0.312 | 1.025 | 0.060 |
| PARP1 | −0.05073 | 0.045 | 0.951 | 0.871 | 1.038 | 0.258 |
| PARP1_Treatment | 0.10513 | 0.063 | 1.111 | 0.982 | 1.257 | 0.095 |

PARP1 is not prognostic and not predictive.

With selected variables

| | | | | | | |
|---|---|---|---|---|---|---|
| Treatment | −0.37983 | 0.300 | 0.684 | 0.380 | 1.232 | 0.206 |
| PARP1 | −0.03038 | 0.044 | 0.970 | 0.889 | 1.058 | 0.492 |
| PARP1_Treatment | 0.06347 | 0.062 | 1.066 | 0.943 | 1.204 | 0.308 |
| T of TNM | 0.35942 | 0.100 | 1.432 | 1.177 | 1.744 | 0.000 |
| N of TNM | 0.40662 | 0.084 | 1.502 | 1.273 | 1.771 | 0.000 |

PARP1 is not prognostic and not predictive.

With adjustment variables

| | | | | | | |
|---|---|---|---|---|---|---|
| Treatment | −0.47480 | 0.307 | 0.622 | 0.341 | 1.135 | 0.122 |
| PARP1 | −0.03205 | 0.045 | 0.968 | 0.887 | 1.057 | 0.475 |
| PARP1_Treatment | 0.07887 | 0.064 | 1.082 | 0.955 | 1.226 | 0.215 |

PARP1 is not prognostic and not predictive.

XPF
ADENOCARCINOMA
Without variables

| | | | | | | |
|---|---|---|---|---|---|---|
| Treatment | −0.04033 | 0.321 | 0.960 | 0.512 | 1.803 | 0.900 |
| XPF | 0.01682 | 0.095 | 1.017 | 0.844 | 1.225 | 0.859 |
| XPF_Treatment | 0.01678 | 0.124 | 1.017 | 0.797 | 1.297 | 0.892 |

XPF is not prognostic and not predictive.

With selected variables

| | | | | | | |
|---|---|---|---|---|---|---|
| Treatment | −0.21475 | 0.321 | 0.807 | 0.430 | 1.513 | 0.503 |
| XPF | 0.06198 | 0.092 | 1.064 | 0.889 | 1.273 | 0.498 |
| XPF_Treatment | 0.06225 | 0.122 | 1.064 | 0.839 | 1.350 | 0.608 |
| T of TNM | 0.38121 | 0.144 | 1.464 | 1.104 | 1.942 | 0.008 |
| Performance Status | 0.30422 | 0.141 | 1.356 | 1.029 | 1.785 | 0.030 |
| Sex | −0.37803 | 0.184 | 0.685 | 0.478 | 0.982 | 0.040 |
| Age | 0.27905 | 0.106 | 1.322 | 1.074 | 1.627 | 0.008 |
| N of TNM | 0.86896 | 0.108 | 2.384 | 1.930 | 2.946 | 0.000 |
| Surgery | −0.45729 | 0.195 | 0.633 | 0.432 | 0.928 | 0.019 |

XPF is not prognostic and not predictive.

With adjustment variables

| | | | | | | |
|---|---|---|---|---|---|---|
| Treatment | −0.17963 | 0.327 | 0.836 | 0.441 | 1.585 | 0.582 |
| XPF | 0.04147 | 0.092 | 1.042 | 0.870 | 1.248 | 0.652 |
| XPF_Treatment | 0.04980 | 0.124 | 1.051 | 0.824 | 1.341 | 0.688 |

XPF is not prognostic and not predictive.

SQUAMOUS CELLS
Without variables

| | | | | | | |
|---|---|---|---|---|---|---|
| Treatment | −0.34944 | 0.262 | 0.705 | 0.422 | 1.179 | 0.183 |
| XPF | −0.08513 | 0.064 | 0.918 | 0.810 | 1.041 | 0.184 |
| XPF_Treatment | 0.09180 | 0.092 | 1.096 | 0.916 | 1.312 | 0.317 |

XPF is not prognostic and not predictive.

With selected variables

| | | | | | | |
|---|---|---|---|---|---|---|
| Treatment | −0.28463 | 0.265 | 0.752 | 0.448 | 1.264 | 0.282 |
| XPF | −0.05223 | 0.064 | 0.949 | 0.837 | 1.077 | 0.416 |
| XPF_Treatment | 0.07132 | 0.092 | 1.074 | 0.896 | 1.287 | 0.441 |
| T of TNM | 0.36048 | 0.101 | 1.434 | 1.177 | 1.747 | 0.000 |
| N of TNM | 0.41357 | 0.084 | 1.512 | 1.283 | 1.783 | 0.000 |

XPF is not prognostic and not predictive.

With adjustment variables

| | | | | | | |
|---|---|---|---|---|---|---|
| Treatment | −0.28065 | 0.269 | 0.755 | 0.446 | 1.280 | 0.297 |
| XPF | −0.04062 | 0.067 | 0.960 | 0.843 | 1.094 | 0.542 |
| XPF_Treatment | 0.06002 | 0.094 | 1.062 | 0.883 | 1.277 | 0.524 |

XPF is not prognostic and not predictive.

ATM
ADENOCARCINOMA
Without variables

| | | | | | | |
|---|---|---|---|---|---|---|
| Treatment | 0.08163 | 0.238 | 1.085 | 0.681 | 1.730 | 0.732 |
| ATM | 0.01752 | 0.065 | 1.018 | 0.896 | 1.156 | 0.788 |
| ATM_Treatment | −0.03984 | 0.091 | 0.961 | 0.804 | 1.149 | 0.662 |

ATM is not prognostic and not predictive.

With selected variables

| | | | | | | |
|---|---|---|---|---|---|---|
| Treatment | −0.02696 | 0.247 | 0.973 | 0.600 | 1.580 | 0.913 |
| ATM | 0.03021 | 0.065 | 1.031 | 0.907 | 1.172 | 0.644 |
| ATM_Treatment | −0.02184 | 0.094 | 0.978 | 0.814 | 1.176 | 0.816 |
| T of TNM | 0.35830 | 0.145 | 1.431 | 1.077 | 1.901 | 0.014 |
| Performance Status | 0.27905 | 0.140 | 1.322 | 1.005 | 1.739 | 0.046 |
| Sex | −0.43521 | 0.181 | 0.647 | 0.453 | 0.924 | 0.016 |
| Age | 0.29283 | 0.107 | 1.340 | 1.087 | 1.653 | 0.006 |
| N of TNM | 0.85260 | 0.109 | 2.346 | 1.894 | 2.905 | 0.000 |
| Surgery | −0.43428 | 0.196 | 0.648 | 0.442 | 0.950 | 0.026 |

ATM is not prognostic and not predictive

With adjustment variables

| | | | | | | |
|---|---|---|---|---|---|---|
| Treatment | 0.01679 | 0.247 | 1.017 | 0.626 | 1.651 | 0.946 |
| ATM | 0.02246 | 0.065 | 1.023 | 0.900 | 1.163 | 0.731 |
| ATM_Treatment | −0.04021 | 0.094 | 0.961 | 0.799 | 1.155 | 0.669 |

ATM is not prognostic and not predictive.

SQUAMOUS CELLS
Without variables

| | | | | | | |
|---|---|---|---|---|---|---|
| Treatment | −0.46895 | 0.206 | 0.626 | 0.418 | 0.937 | 0.023 |
| ATM | −0.07824 | 0.053 | 0.925 | 0.834 | 1.025 | 0.137 |
| ATM_Treatment | 0.16296 | 0.075 | 1.177 | 1.016 | 1.363 | 0.030 |

ATM is not prognostic but predictive.

With selected variables

| | | | | | | |
|---|---|---|---|---|---|---|
| Treatment | −0.51276 | 0.206 | 0.599 | 0.400 | 0.897 | 0.013 |
| ATM | −0.06328 | 0.053 | 0.939 | 0.847 | 1.041 | 0.229 |
| ATM_Treatment | 0.19258 | 0.075 | 1.212 | 1.046 | 1.405 | 0.010 |
| T of TNM | 0.37340 | 0.101 | 1.453 | 1.192 | 1.771 | 0.000 |
| N of TNM | 0.43226 | 0.085 | 1.541 | 1.305 | 1.819 | 0.000 |

ATM is not prognostic but predictive.

With adjustment variables

| | | | | | | |
|---|---|---|---|---|---|---|
| Treatment | −0.51815 | 0.209 | 0.596 | 0.396 | 0.896 | 0.013 |
| ATM | −0.06627 | 0.054 | 0.936 | 0.841 | 1.041 | 0.222 |
| ATM_Treatment | 0.18222 | 0.076 | 1.200 | 1.033 | 1.394 | 0.017 |

ATM is not prognostic but predictive.

BRCA1
ADENOCARCINOMA
Without variables

| | | | | | | |
|---|---|---|---|---|---|---|
| Treatment | 0.30106 | 0.252 | 1.351 | 0.824 | 2.216 | 0.233 |
| BRCA1 | 0.05422 | 0.101 | 1.056 | 0.866 | 1.286 | 0.591 |
| BRCA1_Treatment | −0.19284 | 0.134 | 0.825 | 0.634 | 1.073 | 0.151 |

BRCA1 is not prognostic and not predictive.

With selected variables

| | | | | | | |
|---|---|---|---|---|---|---|
| Treatment | −0.06831 | 0.253 | 0.934 | 0.568 | 1.535 | 0.788 |
| BRCA1 | 0.03338 | 0.094 | 1.034 | 0.860 | 1.243 | 0.723 |
| BRCA1_Treatment | 0.00121 | 0.127 | 1.001 | 0.780 | 1.285 | 0.992 |
| T of TNM | 0.36759 | 0.147 | 1.444 | 1.083 | 1.926 | 0.012 |
| Performance Status | 0.27562 | 0.140 | 1.317 | 1.001 | 1.734 | 0.049 |
| Sex | −0.43211 | 0.181 | 0.649 | 0.455 | 0.926 | 0.017 |
| Age | 0.29345 | 0.107 | 1.341 | 1.087 | 1.654 | 0.006 |
| N of TNM | 0.86350 | 0.109 | 2.371 | 1.917 | 2.934 | 0.000 |
| Surgery | −0.42933 | 0.196 | 0.651 | 0.443 | 0.957 | 0.029 |

BRCA1 is not prognostic and not predictive.

With adjustment variables

| | | | | | | |
|---|---|---|---|---|---|---|
| Treatment | −0.02741 | 0.257 | 0.973 | 0.588 | 1.610 | 0.915 |
| BRCA1 | 0.02344 | 0.094 | 1.024 | 0.851 | 1.232 | 0.804 |
| BRCA1_Treatment | −0.02216 | 0.132 | 0.978 | 0.756 | 1.266 | 0.866 |

BRCA1 is not prognostic and not predictive.

SQUAMOUS CELLS
Without variables

| | | | | | | |
|---|---|---|---|---|---|---|
| Treatment | −0.22329 | 0.194 | 0.800 | 0.546 | 1.171 | 0.251 |
| BRCA1 | −0.01526 | 0.059 | 0.985 | 0.877 | 1.106 | 0.796 |
| BRCA1_Treatment | 0.06698 | 0.084 | 1.069 | 0.907 | 1.260 | 0.424 |

BRCA1 is not prognostic and not predictive.

With selected variables

| | | | | | | |
|---|---|---|---|---|---|---|
| Treatment | −0.23363 | 0.193 | 0.792 | 0.542 | 1.156 | 0.226 |
| BRCA1 | −0.00211 | 0.059 | 0.998 | 0.889 | 1.120 | 0.971 |
| BRCA1_Treatment | 0.07938 | 0.082 | 1.083 | 0.922 | 1.272 | 0.334 |
| T of TNM | 0.37128 | 0.100 | 1.450 | 1.191 | 1.765 | 0.000 |
| N of TNM | 0.41682 | 0.084 | 1.517 | 1.287 | 1.789 | 0.000 |

BRCA1 is not prognostic and not predictive.

With adjustment variables

| | | | | | | |
|---|---|---|---|---|---|---|
| Treatment | −0.24048 | 0.198 | 0.786 | 0.534 | 1.158 | 0.224 |
| BRCA1 | 0.00212 | 0.061 | 1.002 | 0.889 | 1.129 | 0.972 |
| BRCA1_Treatment | 0.06796 | 0.085 | 1.070 | 0.906 | 1.264 | 0.423 |

BRCA1 is not prognostic and not predictive.

ERCC1
ADENOCARCINOMA
Without variables

| | | | | | | |
|---|---|---|---|---|---|---|
| Treatment | 0.04211 | 0.252 | 1.043 | 0.637 | 1.708 | 0.867 |
| ERCC1 | −0.05532 | 0.082 | 0.946 | 0.805 | 1.112 | 0.502 |
| ERCC1_Treatment | −0.01502 | 0.106 | 0.985 | 0.800 | 1.213 | 0.888 |

ERCC1 is not prognostic and not predictive.

With selected variables

| | | | | | | |
|---|---|---|---|---|---|---|
| Treatment | −0.33870 | 0.257 | 0.713 | 0.431 | 1.179 | 0.187 |
| ERCC1 | −0.05265 | 0.079 | 0.949 | 0.813 | 1.107 | 0.502 |
| ERCC1_Treatment | 0.14261 | 0.103 | 1.153 | 0.943 | 1.411 | 0.165 |
| T of TNM | 0.36617 | 0.145 | 1.442 | 1.086 | 1.914 | 0.011 |
| Performance Status | 0.29522 | 0.140 | 1.343 | 1.020 | 1.769 | 0.035 |
| Sex | −0.45662 | 0.181 | 0.633 | 0.445 | 0.902 | 0.011 |
| Age | 0.29745 | 0.107 | 1.346 | 1.091 | 1.661 | 0.005 |
| N of TNM | 0.87889 | 0.109 | 2.408 | 1.945 | 2.982 | 0.000 |
| Surgery | −0.47903 | 0.199 | 0.619 | 0.420 | 0.914 | 0.016 |

ERCC1 is not prognostic and not predictive.

With adjustment variables

| | | | | | | |
|---|---|---|---|---|---|---|
| Treatment | −0.32825 | 0.259 | 0.720 | 0.433 | 1.197 | 0.205 |
| ERCC1 | 0.06000 | 0.079 | 0.942 | 0.807 | 1.099 | 0.445 |
| ERCC1_Treatment | 0.14215 | 0.105 | 1.153 | 0.938 | 1.416 | 0.176 |

ERCC1 is not prognostic and not predictive.

SQUAMOUS CELLS
Without variables

| | | | | | | |
|---|---|---|---|---|---|---|
| Treatment | −0.32173 | 0.203 | 0.725 | 0.487 | 1.080 | 0.114 |
| ERCC1 | −0.05422 | 0.046 | 0.947 | 0.865 | 1.037 | 0.243 |
| ERCC1_Treatment | 0.07863 | 0.061 | 1.082 | 0.961 | 1.218 | 0.195 |

ERCC1 is not prognostic and not predictive.

With selected variables

| | | | | | | |
|---|---|---|---|---|---|---|
| Treatment | −0.30958 | 0.204 | 0.734 | 0.492 | 1.093 | 0.128 |
| ERCC1 | 0.04144 | 0.047 | 0.959 | 0.876 | 1.051 | 0.375 |
| ERCC1_Treatment | 0.07759 | 0.061 | 1.081 | 0.960 | 1.217 | 0.200 |
| T of TNM | 0.36460 | 0.101 | 1.440 | 1.181 | 1.755 | 0.000 |
| N of TNM | 0.41489 | 0.084 | 1.514 | 1.284 | 1.786 | 0.000 |

ERCC1 is not prognostic and not predictive.

With adjustment variables

| | | | | | | |
|---|---|---|---|---|---|---|
| Treatment | −0.35826 | 0.208 | 0.699 | 0.465 | 1.050 | 0.085 |
| ERCC1 | −0.05001 | 0.048 | 0.951 | 0.867 | 1.044 | 0.293 |
| ERCC1_Treatment | 0.08540 | 0.062 | 1.089 | 0.964 | 1.230 | 0.170 |

ERCC1 is not prognostic and not predictive.

Overall Survival
MSH2
ADENOCARCINOMA
Without variables

| | | | | | | |
|---|---|---|---|---|---|---|
| Treatment | 0.33568 | 0.264 | 1.399 | 0.834 | 2.346 | 0.203 |
| MSH2 | 0.00238 | 0.107 | 1.002 | 0.812 | 1.237 | 0.982 |
| MSH2_Treatment | −0.19218 | 0.158 | 0.825 | 0.606 | 1.124 | 0.223 |

MSH2 is not prognostic and not predictive.

With selected variables

| | | | | | | |
|---|---|---|---|---|---|---|
| Treatment | −0.05501 | 0.274 | 0.946 | 0.554 | 1.618 | 0.841 |
| MSH2 | −0.05383 | 0.108 | 0.948 | 0.767 | 1.171 | 0.618 |
| MSH2_Treatment | −0.03710 | 0.160 | 0.964 | 0.704 | 1.319 | 0.817 |
| T of TNM | 0.30253 | 0.150 | 1.353 | 1.009 | 1.816 | 0.044 |
| Sex | −0.38324 | 0.191 | 0.682 | 0.469 | 0.991 | 0.045 |
| Age | 0.38231 | 0.108 | 1.466 | 1.186 | 1.812 | 0.000 |
| N of TNM | 0.70819 | 0.108 | 2.030 | 1.644 | 2.508 | 0.000 |
| Surgery | −0.41318 | 0.199 | 0.662 | 0.448 | 0.977 | 0.038 |

MSH2 is not prognostic and not predictive.

With adjustment variables

| | | | | | | |
|---|---|---|---|---|---|---|
| Treatment | −0.05262 | 0.278 | 0.949 | 0.550 | 1.637 | 0.850 |
| MSH2 | −0.09372 | 0.108 | 0.911 | 0.738 | 1.124 | 0.383 |
| MSH2_Treatment | −0.03580 | 0.163 | 0.965 | 0.701 | 1.329 | 0.826 |

MSH2 is not prognostic and not predictive.

SQUAMOUS CELL
Without variables

| | | | | | | |
|---|---|---|---|---|---|---|
| Treatment | −0.40888 | 0.201 | 0.664 | 0.448 | 0.984 | 0.042 |
| MSH2 | −0.10265 | 0.056 | 0.902 | 0.808 | 1.008 | 0.068 |
| MSH2_Treatment | 0.14361 | 0.081 | 1.154 | 0.985 | 1.352 | 0.075 |

MSH2 is not prognostic and not predictive.

With selected variables

| | | | | | | |
|---|---|---|---|---|---|---|
| Treatment | −0.39595 | 0.199 | 0.673 | 0.455 | 0.995 | 0.047 |
| MSH2 | −0.09121 | 0.055 | 0.913 | 0.819 | 1.018 | 0.100 |
| MSH2_Treatment | 0.14075 | 0.080 | 1.151 | 0.985 | 1.346 | 0.078 |
| T of TNM | 0.38788 | 0.102 | 1.474 | 1.207 | 1.800 | 0.000 |
| N of TNM | 0.42650 | 0.085 | 1.532 | 1.298 | 1.808 | 0.000 |

MSH2 is not prognostic and not predictive.

With adjustment variables

| | | | | | | |
|---|---|---|---|---|---|---|
| Treatment | −0.40510 | 0.200 | 0.667 | 0.451 | 0.987 | 0.043 |
| MSH2 | −0.09538 | 0.056 | 0.909 | 0.815 | 1.014 | 0.088 |
| MSH2_Treatment | 0.13417 | 0.080 | 1.144 | 0.978 | 1.337 | 0.093 |

MSH2 is not prognostic and not predictive.

P53
ADENOCARCINOMA
Without variables

| | | | | | | |
|---|---|---|---|---|---|---|
| Treatment | 0.12082 | 0.205 | 1.128 | 0.756 | 1.685 | 0.555 |
| P53 | 0.00396 | 0.048 | 1.004 | 0.914 | 1.103 | 0.934 |
| P53_Treatment | −0.03050 | 0.074 | 0.970 | 0.839 | 1.122 | 0.681 |

P53 is not prognostic and not predictive.

With selected variables

| | | | | | | |
|---|---|---|---|---|---|---|
| Treatment | −0.09484 | 0.208 | 0.910 | 0.605 | 1.366 | 0.648 |
| P53 | −0.03598 | 0.049 | 0.965 | 0.877 | 1.061 | 0.459 |
| P53_Treatment | 0.08261 | 0.075 | 1.086 | 0.938 | 1.258 | 0.270 |
| T of TNM | 0.43793 | 0.141 | 1.550 | 1.176 | 2.042 | 0.002 |
| Age | 0.34813 | 0.106 | 1.416 | 1.150 | 1.744 | 0.001 |
| N of TNM | 0.75156 | 0.105 | 2.120 | 1.725 | 2.606 | 0.000 |

P53 is not prognostic and not predictive.

With adjustment variables

| | | | | | | |
|---|---|---|---|---|---|---|
| Treatment | −0.18061 | 0.220 | 0.835 | 0.543 | 1.284 | 0.411 |
| P53 | −0.02777 | 0.049 | 0.973 | 0.883 | 1.071 | 0.571 |
| P53_Treatment | 0.05621 | 0.075 | 1.058 | 0.913 | 1.226 | 0.455 |

P53 is not prognostic and not predictive.

SQUAMOUS CELLS
Without variables

| | | | | | | |
|---|---|---|---|---|---|---|
| Treatment | −0.42089 | 0.171 | 0.656 | 0.469 | 0.919 | 0.014 |
| P53 | −0.07351 | 0.029 | 0.929 | 0.877 | 0.984 | 0.013 |
| P53_Treatment | 0.10115 | 0.041 | 1.106 | 1.021 | 1.199 | 0.014 |

P53 is prognostic and predictive.

With selected variables

| | | | | | | |
|---|---|---|---|---|---|---|
| Treatment | −0.44091 | 0.173 | 0.643 | 0.459 | 0.903 | 0.011 |
| P53 | −0.06783 | 0.030 | 0.934 | 0.882 | 0.990 | 0.022 |
| P53_Treatment | 0.10569 | 0.041 | 1.111 | 1.026 | 1.205 | 0.010 |
| T of TNM | 0.37429 | 0.101 | 1.454 | 1.192 | 1.774 | 0.000 |
| Performance Status | 0.20312 | 0.100 | 1.225 | 1.006 | 1.492 | 0.043 |
| N of TNM | 0.42546 | 0.085 | 1.530 | 1.295 | 1.808 | 0.000 |

P53 is prognostic and predictive.

With adjustment variables

| | | | | | | |
|---|---|---|---|---|---|---|
| Treatment | −0.42772 | 0.176 | 0.652 | 0.462 | 0.920 | 0.015 |
| P53 | −0.07032 | 0.030 | 0.932 | 0.879 | 0.989 | 0.019 |
| P53_Treatment | 0.09655 | 0.041 | 1.101 | 1.015 | 1.195 | 0.020 |

P53 is prognostic and predictive.

PARP1
ADENOCARCINOMA
Without variables

| | | | | | | |
|---|---|---|---|---|---|---|
| Treatment | 0.10579 | 0.363 | 1.112 | 0.546 | 2.263 | 0.770 |
| PARP1 | 0.00785 | 0.059 | 1.008 | 0.898 | 1.132 | 0.894 |
| PARP1_Treatment | −0.00796 | 0.079 | 0.992 | 0.850 | 1.158 | 0.920 |

PARP1 is not prognostic and not predictive.

With selected variables

| | | | | | | |
|---|---|---|---|---|---|---|
| Treatment | 0.03712 | 0.373 | 1.038 | 0.500 | 2.154 | 0.921 |
| PARP1 | 0.01405 | 0.060 | 1.014 | 0.902 | 1.140 | 0.814 |
| PARP1_Treatment | −0.01139 | 0.081 | 0.989 | 0.843 | 1.160 | 0.889 |
| T of TNM | 0.36196 | 0.144 | 1.436 | 1.082 | 1.906 | 0.012 |
| Age | 0.37896 | 0.107 | 1.461 | 1.183 | 1.803 | 0.000 |
| N of TNM | 0.68132 | 0.106 | 1.976 | 1.605 | 2.434 | 0.000 |
| Surgery | −0.39718 | 0.199 | 0.672 | 0.455 | 0.993 | 0.046 |

PARP1 is not prognostic and not predictive.

With adjustment variables

| | | | | | | |
|---|---|---|---|---|---|---|
| Treatment | 0.04162 | 0.388 | 1.042 | 0.488 | 2.229 | 0.915 |
| PARP1 | −0.02670 | 0.064 | 0.974 | 0.859 | 1.104 | 0.677 |
| PARP1_Treatment | −0.02685 | 0.085 | 0.974 | 0.824 | 1.150 | 0.752 |

PARP1 is not prognostic and not predictive.

SQUAMOUS CELLS
Without variables

| | | | | | | |
|---|---|---|---|---|---|---|
| Treatment | −0.55899 | 0.304 | 0.572 | 0.315 | 1.038 | 0.066 |
| PARP1 | −0.04630 | 0.045 | 0.955 | 0.875 | 1.042 | 0.300 |
| PARP1_Treatment | 0.10162 | 0.063 | 1.107 | 0.978 | 1.253 | 0.109 |

PARP1 is not prognostic and not predictive.

With selected variables

| | | | | | | |
|---|---|---|---|---|---|---|
| Treatment | −0.37551 | 0.302 | 0.687 | 0.380 | 1.242 | 0.214 |
| PARP1 | −0.02590 | 0.044 | 0.974 | 0.894 | 1.062 | 0.557 |
| PARP1_Treatment | 0.06058 | 0.063 | 1.062 | 0.940 | 1.201 | 0.334 |
| T of TNM | 0.38609 | 0.102 | 1.471 | 1.206 | 1.795 | 0.000 |
| N of TNM | 0.41640 | 0.085 | 1.516 | 1.285 | 1.790 | 0.000 |

PARP1 is not prognostic and not predictive.

With adjustment variables

| | | | | | | |
|---|---|---|---|---|---|---|
| Treatment | −0.47677 | 0.309 | 0.621 | 0.339 | 1.137 | 0.122 |
| PARP1 | −0.03153 | 0.045 | 0.969 | 0.888 | 1.057 | 0.479 |
| PARP1_Treatment | 0.07841 | 0.064 | 1.082 | 0.954 | 1.226 | 0.220 |

PARP1 is not prognostic and not predictive.

XPF
ADENOCARCINOMA
Without variables

| | | | | | | |
|---|---|---|---|---|---|---|
| Treatment | −0.10572 | 0.341 | 0.900 | 0.462 | 1.754 | 0.756 |
| XPF | −0.00898 | 0.103 | 0.991 | 0.809 | 1.214 | 0.931 |
| XPF_Treatment | 0.07701 | 0.132 | 1.080 | 0.834 | 1.398 | 0.559 |

XPF is not prognostic and not predictive.

With selected variables

| | | | | | | |
|---|---|---|---|---|---|---|
| Treatment | −0.37312 | 0.342 | 0.689 | 0.352 | 1.346 | 0.275 |
| XPF | 0.04537 | 0.101 | 1.046 | 0.859 | 1.274 | 0.652 |
| XPF_Treatment | 0.15328 | 0.129 | 1.166 | 0.904 | 1.502 | 0.237 |
| T of TNM | 0.42031 | 0.148 | 1.522 | 1.140 | 2.033 | 0.004 |
| Age | 0.36549 | 0.108 | 1.441 | 1.167 | 1.779 | 0.001 |
| N of TNM | 0.71641 | 0.107 | 2.047 | 1.660 | 2.524 | 0.000 |
| Surgery | −0.44124 | 0.199 | 0.643 | 0.435 | 0.950 | 0.027 |

XPF is not prognostic and not predictive.

With adjustment variables

| | | | | | | |
|---|---|---|---|---|---|---|
| Treatment | −0.46446 | 0.356 | 0.628 | 0.313 | 1.262 | 0.192 |
| XPF | −0.01388 | 0.102 | 0.986 | 0.808 | 1.204 | 0.891 |
| XPF_Treatment | 0.16478 | 0.133 | 1.179 | 0.909 | 1.530 | 0.215 |

XPF is not prognostic and not predictive.

SQUAMOUS CELLS
Without variables

| | | | | | | |
|---|---|---|---|---|---|---|
| Treatment | −0.27634 | 0.265 | 0.759 | 0.451 | 1.276 | 0.298 |
| XPF | −0.07255 | 0.065 | 0.930 | 0.819 | 1.056 | 0.263 |
| XPF_Treatment | 0.06087 | 0.093 | 1.063 | 0.885 | 1.276 | 0.513 |

XPF is not prognostic and not predictive.

With selected variables

| | | | | | | |
|---|---|---|---|---|---|---|
| Treatment | −0.19890 | 0.268 | 0.820 | 0.484 | 1.387 | 0.458 |
| XPF | −0.03602 | 0.065 | 0.965 | 0.849 | 1.096 | 0.580 |
| XPF_Treatment | 0.03432 | 0.094 | 1.035 | 0.860 | 1.245 | 0.716 |
| T of TNM | 0.38564 | 0.102 | 1.471 | 1.205 | 1.795 | 0.000 |
| N of TNM | 0.42360 | 0.084 | 1.527 | 1.294 | 1.802 | 0.000 |

XPF is not prognostic and not predictive.

With adjustment variables

| | | | | | | |
|---|---|---|---|---|---|---|
| Treatment | −0.20690 | 0.273 | 0.813 | 0.476 | 1.389 | 0.449 |
| XPF | −0.02620 | 0.067 | 0.974 | 0.854 | 1.112 | 0.698 |
| XPF_Treatment | 0.02946 | 0.096 | 1.030 | 0.853 | 1.243 | 0.759 |

XPF is not prognostic and not predictive.

ATM
ADENOCARCINOMA
Without variables

| | | | | | | |
|---|---|---|---|---|---|---|
| Treatment | 0.13287 | 0.247 | 1.142 | 0.704 | 1.852 | 0.590 |
| ATM | −0.00783 | 0.072 | 0.992 | 0.862 | 1.142 | 0.913 |
| ATM_Treatment | −0.02799 | 0.097 | 0.972 | 0.804 | 1.176 | 0.773 |

ATM is not prognostic and not predictive.

With selected variables

| | | | | | | |
|---|---|---|---|---|---|---|
| Treatment | 0.00405 | 0.252 | 1.004 | 0.612 | 1.647 | 0.987 |
| ATM | 0.02465 | 0.072 | 1.025 | 0.891 | 1.179 | 0.730 |
| ATM_Treatment | −0.00717 | 0.098 | 0.993 | 0.819 | 1.204 | 0.942 |
| T of TNM | 0.37351 | 0.147 | 1.453 | 1.090 | 1.937 | 0.011 |
| Age | 0.38771 | 0.109 | 1.474 | 1.190 | 1.825 | 0.000 |
| N of TNM | 0.67617 | 0.106 | 1.966 | 1.596 | 2.422 | 0.000 |
| Surgery | −0.40349 | 0.200 | 0.668 | 0.452 | 0.988 | 0.043 |

ATM is not prognostic and not predictive.

With adjustment variables

| | | | | | | |
|---|---|---|---|---|---|---|
| Treatment | 0.03272 | 0.258 | 1.033 | 0.623 | 1.713 | 0.899 |
| ATM | 0.01663 | 0.072 | 1.017 | 0.884 | 1.170 | 0.816 |
| ATM_Treatment | −0.06045 | 0.101 | 0.941 | 0.773 | 1.146 | 0.548 |

ATM is not prognostic and not predictive.

SQUAMOUS CELLS
Without variables

| | | | | | | |
|---|---|---|---|---|---|---|
| Treatment | −0.41773 | 0.207 | 0.659 | 0.439 | 0.989 | 0.044 |
| ATM | −0.06045 | 0.052 | 0.941 | 0.850 | 1.043 | 0.247 |
| ATM_Treatment | 0.13845 | 0.076 | 1.148 | 0.990 | 1.332 | 0.067 |

ATM is not prognostic and not predictive.

With selected variables

| | | | | | | |
|---|---|---|---|---|---|---|
| Treatment | −0.44557 | 0.207 | 0.640 | 0.427 | 0.961 | 0.032 |
| ATM | −0.04200 | 0.052 | 0.959 | 0.866 | 1.061 | 0.418 |
| ATM_Treatment | 0.15897 | 0.075 | 1.172 | 1.011 | 1.359 | 0.035 |
| T of TNM | 0.39569 | 0.102 | 1.485 | 1.216 | 1.814 | 0.000 |
| N of TNM | 0.43663 | 0.085 | 1.547 | 1.310 | 1.827 | 0.000 |

ATM is not prognostic but predictive.

With adjustment variables

| | | | | | | |
|---|---|---|---|---|---|---|
| Treatment | −0.46940 | 0.210 | 0.625 | 0.414 | 0.944 | 0.025 |
| ATM | −0.05105 | 0.054 | 0.950 | 0.855 | 1.056 | 0.344 |
| ATM_Treatment | 0.15871 | 0.077 | 1.172 | 1.008 | 1.362 | 0.039 |

ATM is not prognostic but predictive.

BRCA1
ADENOCARCINOMA
Without variables

| | | | | | | |
|---|---|---|---|---|---|---|
| Treatment | 0.33010 | 0.265 | 1.391 | 0.828 | 2.337 | 0.213 |
| BRCA1 | 0.09317 | 0.106 | 1.098 | 0.891 | 1.352 | 0.381 |
| BRCA1_Treatment | −0.16953 | 0.138 | 0.844 | 0.644 | 1.106 | 0.218 |

BRCA1 is not prognostic and not predictive.

With selected variables

| | | | | | | |
|---|---|---|---|---|---|---|
| Treatment | −0.04711 | 0.260 | 0.954 | 0.573 | 1.589 | 0.856 |
| BRCA1 | 0.11078 | 0.097 | 1.117 | 0.924 | 1.350 | 0.252 |
| BRCA1_Treatment | 0.02661 | 0.128 | 1.027 | 0.799 | 1.321 | 0.836 |
| T of TNM | 0.43188 | 0.151 | 1.540 | 1.146 | 2.069 | 0.004 |
| Age | 0.39963 | 0.107 | 1.491 | 1.208 | 1.841 | 0.000 |
| N of TNM | 0.70787 | 0.107 | 2.030 | 1.645 | 2.505 | 0.000 |
| Surgery | −0.40098 | 0.200 | 0.670 | 0.452 | 0.992 | 0.045 |

BRCA1 is not prognostic and not predictive.

With adjustment variables

| | | | | | | |
|---|---|---|---|---|---|---|
| Treatment | −0.11136 | 0.274 | 0.895 | 0.523 | 1.532 | 0.685 |
| BRCA1 | 0.07215 | 0.099 | 1.075 | 0.885 | 1.305 | 0.466 |
| BRCA1_Treatment | 0.02203 | 0.134 | 1.022 | 0.787 | 1.328 | 0.869 |

BRCA1 is not prognostic and not predictive.

SQUAMOUS CELLS
Without variables

| | | | | | | |
|---|---|---|---|---|---|---|
| Treatment | −0.15971 | 0.197 | 0.852 | 0.579 | 1.255 | 0.418 |
| BRCA1 | −0.00031 | 0.059 | 1.000 | 0.890 | 1.123 | 0.996 |
| BRCA1_Treatment | 0.02767 | 0.086 | 1.028 | 0.869 | 1.216 | 0.747 |

BRCA1 is not prognostic and not predictive.

With selected variables

| | | | | | | |
|---|---|---|---|---|---|---|
| Treatment | −0.18480 | 0.197 | 0.831 | 0.566 | 1.222 | 0.347 |
| BRCA1 | 0.00810 | 0.059 | 1.008 | 0.897 | 1.133 | 0.891 |
| BRCA1_Treatment | 0.04657 | 0.085 | 1.048 | 0.888 | 1.236 | 0.582 |
| T of TNM | 0.39412 | 0.102 | 1.483 | 1.215 | 1.810 | 0.000 |
| N of TNM | 0.42593 | 0.084 | 1.531 | 1.298 | 1.806 | 0.000 |

BRCA1 is not prognostic and not predictive.

With adjustment variables

| | | | | | | |
|---|---|---|---|---|---|---|
| Treatment | −0.20006 | 0.201 | 0.819 | 0.552 | 1.214 | 0.319 |
| BRCA1 | 0.00660 | 0.061 | 1.007 | 0.893 | 1.135 | 0.914 |
| BRCA1_Treatment | 0.04214 | 0.087 | 1.043 | 0.880 | 1.236 | 0.627 |

BRCA1 is not prognostic and not predictive.

ERCC1
ADENOCARCINOMA
Without variables

| | | | | | | |
|---|---|---|---|---|---|---|
| Treatment | 0.03902 | 0.266 | 1.040 | 0.617 | 1.753 | 0.884 |
| ERCC1 | −0.07424 | 0.093 | 0.928 | 0.774 | 1.114 | 0.424 |
| ERCC1_Treatment | 0.02386 | 0.116 | 1.024 | 0.817 | 1.284 | 0.836 |

ERCC1 is not prognostic and not predictive.

With selected variables

| | | | | | | |
|---|---|---|---|---|---|---|
| Treatment | −0.26774 | 0.269 | 0.765 | 0.452 | 1.296 | 0.319 |
| ERCC1 | −0.04389 | 0.088 | 0.957 | 0.805 | 1.138 | 0.620 |
| ERCC1_Treatment | 0.14028 | 0.113 | 1.151 | 0.922 | 1.435 | 0.214 |
| T of TNM | 0.38025 | 0.146 | 1.463 | 1.099 | 1.946 | 0.009 |
| Age | 0.39092 | 0.108 | 1.478 | 1.197 | 1.826 | 0.000 |
| N of TNM | 0.69600 | 0.106 | 2.006 | 1.629 | 2.470 | 0.000 |
| Surgery | −0.44523 | 0.203 | 0.641 | 0.431 | 0.953 | 0.028 |

ERCC1 is not prognostic and not predictive.

With adjustment variables

| | | | | | | |
|---|---|---|---|---|---|---|
| Treatment | −0.37284 | 0.278 | 0.689 | 0.399 | 1.188 | 0.180 |
| ERCC1 | −0.06336 | 0.088 | 0.939 | 0.789 | 1.116 | 0.474 |
| ERCC1_Treatment | 0.15726 | 0.116 | 1.170 | 0.932 | 1.469 | 0.175 |

ERCC1 is not prognostic and not predictive.

SQUAMOUS CELLS
Without variables

| | | | | | | |
|---|---|---|---|---|---|---|
| Treatment | −0.25756 | 0.206 | 0.773 | 0.517 | 1.157 | 0.210 |
| ERCC1 | −0.04055 | 0.046 | 0.960 | 0.877 | 1.051 | 0.380 |
| ERCC1_Treatment | 0.05270 | 0.061 | 1.054 | 0.935 | 1.189 | 0.391 |

ERCC1 is not prognostic and not predictive.

With selected variables

| | | | | | | |
|---|---|---|---|---|---|---|
| Treatment | −0.24663 | 0.206 | 0.781 | 0.522 | 1.171 | 0.232 |
| ERCC1 | −0.02723 | 0.047 | 0.973 | 0.888 | 1.066 | 0.560 |
| ERCC1_Treatment | 0.05107 | 0.061 | 1.052 | 0.933 | 1.187 | 0.406 |
| T of TNM | 0.38988 | 0.102 | 1.477 | 1.209 | 1.804 | 0.000 |
| N of TNM | 0.42411 | 0.084 | 1.528 | 1.295 | 1.803 | 0.000 |

ERCC1 is not prognostic and not predictive.

With adjustment variables

| | | | | | | |
|---|---|---|---|---|---|---|
| Treatment | −0.31398 | 0.211 | 0.731 | 0.483 | 1.104 | 0.136 |
| ERCC1 | −0.04129 | 0.048 | 0.960 | 0.874 | 1.054 | 0.388 |
| ERCC1_Treatment | 0.06740 | 0.063 | 1.070 | 0.945 | 1.211 | 0.286 |

ERCC1 is not prognostic and not predictive.

pMK2
Disease Free Survival
MEAN Q SCORE PMK2 CYTOPLASM
Adenocarcinoma
Without variables

| | | | | | | |
|---|---|---|---|---|---|---|
| Treatment | 0.15915 | 0.190 | 1.173 | 0.808 | 1.702 | 0.402 |
| PMK2MC | 0.17810 | 0.216 | 1.195 | 0.782 | 1.825 | 0.410 |
| PMK2MC_Treatment | −0.43566 | 0.302 | 0.647 | 0.358 | 1.169 | 0.149 |

Not Prognostic. Not predictive.

With selected variables

| | | | | | | |
|---|---|---|---|---|---|---|
| Treatment | −0.13434 | 0.202 | 0.874 | 0.589 | 1.299 | 0.506 |
| PMK2MC | −0.24886 | 0.210 | 0.780 | 0.517 | 1.176 | 0.235 |
| PMK2MC_Treatment | 0.09205 | 0.330 | 1.096 | 0.575 | 2.092 | 0.780 |
| Disease Stage | 0.01811 | 0.210 | 1.018 | 0.675 | 1.536 | 0.931 |
| T of TNM | 0.33623 | 0.168 | 1.400 | 1.006 | 1.947 | 0.046 |
| Performance Status | 0.26314 | 0.141 | 1.301 | 0.988 | 1.714 | 0.061 |
| Sex | −0.45435 | 0.180 | 0.635 | 0.446 | 0.904 | 0.012 |
| Age | 0.30636 | 0.109 | 1.358 | 1.097 | 1.682 | 0.005 |
| N of TNM | 0.87508 | 0.201 | 2.399 | 1.617 | 3.560 | 0.000 |
| Surgery | −0.43175 | 0.197 | 0.649 | 0.441 | 0.956 | 0.029 |

Not Prognostic. Not predictive.

With adjustment variables

| | | | | | | |
|---|---|---|---|---|---|---|
| Treatment | −0.14855 | 0.203 | 0.862 | 0.579 | 1.283 | 0.464 |
| PMK2MC | −0.27985 | 0.210 | 0.756 | 0.500 | 1.142 | 0.183 |
| PMK2MC_Treatment | 0.15099 | 0.331 | 1.163 | 0.608 | 2.224 | 0.648 |

Not Prognostic. Not predictive.

Squamous Cells
Without variables

| | | | | | | |
|---|---|---|---|---|---|---|
| Treatment | −0.16681 | 0.139 | 0.846 | 0.644 | 1.112 | 0.231 |
| PMK2MC | −0.13114 | 0.121 | 0.877 | 0.693 | 1.111 | 0.277 |
| PMK2MC_Treatment | 0.12591 | 0.139 | 1.134 | 0.864 | 1.490 | 0.365 |

Not Prognostic. Not predictive.

With selected variables

| | | | | | | |
|---|---|---|---|---|---|---|
| Treatment | −0.18989 | 0.141 | 0.827 | 0.628 | 1.090 | 0.177 |
| PMK2MC | −0.19704 | 0.120 | 0.821 | 0.649 | 1.038 | 0.100 |
| PMK2MC_Treatment | 0.18456 | 0.140 | 1.203 | 0.914 | 1.583 | 0.188 |
| Disease Stage | −0.04425 | 0.139 | 0.957 | 0.729 | 1.255 | 0.749 |
| T of TNM | 0.37129 | 0.150 | 1.450 | 1.079 | 1.947 | 0.014 |
| Performance Status | 0.16598 | 0.099 | 1.181 | 0.972 | 1.434 | 0.094 |
| Sex | −0.26164 | 0.214 | 0.770 | 0.506 | 1.172 | 0.222 |
| N of TNM | 0.47035 | 0.117 | 1.601 | 1.271 | 2.015 | 0.000 |

Not Prognostic. Not predictive.

With adjustment variables

| | | | | | | |
|---|---|---|---|---|---|---|
| Treatment | −0.21831 | 0.141 | 0.804 | 0.610 | 1.059 | 0.121 |
| PMK2MC | −0.21741 | 0.119 | 0.805 | 0.638 | 1.015 | 0.067 |
| PMK2MC_Treatment | 0.18706 | 0.139 | 1.206 | 0.919 | 1.582 | 0.177 |

Not Prognostic. Not predictive.

MEAN Q SCORE OF PMK2 CYTOPLASM DIVIDED BY NUCLEAR
Adenocarcinoma
Without variables

| | | | | | | |
|---|---|---|---|---|---|---|
| Treatment | −0.00297 | 0.310 | 0.997 | 0.543 | 1.830 | 0.992 |
| PMK2MCNp | 27.38317 | 102.0 | 78E10 | 0.000 | 55E97 | 0.788 |
| PMK2MCNp_Treatment | 5.57441 | 145.0 | 263.6 | 0.000 | 6E125 | 0.969 |

Not Prognostic. Not predictive.

With selected variables

| | | | | | | |
|---|---|---|---|---|---|---|
| Treatment | −0.18985 | 0.334 | 0.827 | 0.430 | 1.591 | 0.570 |
| PMK2MCNp | −105.397 | 109.3 | 0.000 | 0.000 | 18E46 | 0.335 |
| PMK2MCNp_Treatment | 54.10423 | 160.6 | 31E22 | 0.000 | 2E160 | 0.736 |
| Disease Stage | 0.00451 | 0.210 | 1.005 | 0.665 | 1.517 | 0.983 |
| T of TNM | 0.35090 | 0.169 | 1.420 | 1.019 | 1.980 | 0.038 |
| Performance Status | 0.26831 | 0.140 | 1.308 | 0.993 | 1.721 | 0.056 |
| Sex | −0.44879 | 0.180 | 0.638 | 0.448 | 0.909 | 0.013 |
| Age | 0.29999 | 0.108 | 1.350 | 1.093 | 1.668 | 0.005 |
| N of TNM | 0.88055 | 0.203 | 2.412 | 1.622 | 3.588 | 0.000 |
| Surgery | −0.42810 | 0.196 | 0.652 | 0.444 | 0.957 | 0.029 |

Not Prognostic. Not predictive.

With adjustment variables

| | | | | | | |
|---|---|---|---|---|---|---|
| Treatment | −0.23527 | 0.337 | 0.790 | 0.408 | 1.530 | 0.485 |
| PMK2MCNp | −115.898 | 110.0 | 0.000 | 0.000 | 2E43 | 0.292 |
| PMK2MCNp_Treatment | 83.41330 | 162.1 | 17E35 | 0.000 | 2E174 | 0.607 |

Not Prognostic. Not predictive.

Squamous Cells
Without variables

| | | | | | | |
|---|---|---|---|---|---|---|
| Treatment | −0.28055 | 0.238 | 0.755 | 0.474 | 1.203 | 0.238 |
| PMK2MCNp | −38.9861 | 64.22 | 0.000 | 0.000 | 54E36 | 0.544 |
| PMK2MCNp_Treatment | 66.58625 | 78.73 | 83E27 | 0.000 | 86E94 | 0.398 |

Not Prognostic. Not predictive.

With selected variables

| | | | | | | |
|---|---|---|---|---|---|---|
| Treatment | −0.38374 | 0.240 | 0.681 | 0.425 | 1.092 | 0.111 |
| PMK2MCNp | −76.0522 | 64.32 | 0.000 | 0.000 | 53E20 | 0.237 |
| PMK2MCNp_Treatment | 110.1388 | 80.18 | 68E46 | 0.000 | 1E116 | 0.170 |
| Disease Stage | −0.05555 | 0.138 | 0.946 | 0.721 | 1.241 | 0.688 |
| T of TNM | 0.37049 | 0.150 | 1.448 | 1.079 | 1.944 | 0.014 |
| Performance Status | 0.16797 | 0.098 | 1.183 | 0.975 | 1.435 | 0.088 |
| Sex | −0.25287 | 0.214 | 0.777 | 0.510 | 1.182 | 0.238 |
| N of TNM | 0.47471 | 0.118 | 1.608 | 1.276 | 2.025 | 0.000 |

Not Prognostic. Not predictive.

With adjustment variables

| | | | | | | |
|---|---|---|---|---|---|---|
| Treatment | −0.44175 | 0.240 | 0.643 | 0.402 | 1.029 | 0.065 |
| PMK2MCNp | −80.7374 | 63.42 | 0.000 | 0.000 | 83E17 | 0.203 |
| PMK2MCNp_Treatment | 121.1232 | 79.68 | 4E52 | 0.000 | 3E120 | 0.128 |

Not Prognostic. Not predictive.

MEAN Q SCORE PMK2 NUCLEAR
Adenocarcinoma
Without variables

| | | | | | | |
|---|---|---|---|---|---|---|
| Treatment | 0.13547 | 0.230 | 1.145 | 0.729 | 1.798 | 0.556 |
| PMK2MN | 0.01015 | 0.097 | 1.010 | 0.835 | 1.222 | 0.917 |
| PMK2MN_Treatment | −0.09340 | 0.121 | 0.911 | 0.719 | 1.154 | 0.440 |

Not Prognostic. Not predictive.

With selected variables

| | | | | | | |
|---|---|---|---|---|---|---|
| Treatment | −0.20308 | 0.239 | 0.816 | 0.511 | 1.304 | 0.396 |
| PMK2MN | −0.09994 | 0.088 | 0.905 | 0.762 | 1.075 | 0.256 |
| PMK2MN_Treatment | 0.08321 | 0.122 | 1.087 | 0.855 | 1.381 | 0.497 |
| Disease Stage | 0.01515 | 0.211 | 1.015 | 0.671 | 1.537 | 0.943 |
| T of TNM | 0.33159 | 0.170 | 1.393 | 0.999 | 1.942 | 0.051 |
| Performance Status | 0.27937 | 0.140 | 1.322 | 1.004 | 1.741 | 0.047 |
| Sex | −0.45259 | 0.180 | 0.636 | 0.447 | 0.905 | 0.012 |
| Age | 0.30009 | 0.109 | 1.350 | 1.090 | 1.672 | 0.006 |
| N of TNM | 0.86679 | 0.202 | 2.379 | 1.601 | 3.536 | 0.000 |
| Surgery | −0.43449 | 0.199 | 0.648 | 0.438 | 0.957 | 0.029 |

Not Prognostic. Not predictive.

With adjustment variables

| | | | | | | |
|---|---|---|---|---|---|---|
| Treatment | −0.20509 | 0.239 | 0.815 | 0.510 | 1.302 | 0.391 |
| PMK2MN | −0.11285 | 0.087 | 0.893 | 0.753 | 1.060 | 0.197 |
| PMK2MN_Treatment | 0.09009 | 0.123 | 1.094 | 0.859 | 1.394 | 0.465 |

Not Prognostic. Not predictive.

Squamous Cells
Without variables

| | | | | | | |
|---|---|---|---|---|---|---|
| Treatment | −0.23708 | 0.160 | 0.789 | 0.577 | 1.079 | 0.138 |
| PMK2MN | −0.09386 | 0.064 | 0.910 | 0.803 | 1.033 | 0.145 |
| PMK2MN_Treatment | 0.10203 | 0.081 | 1.107 | 0.945 | 1.298 | 0.208 |

Not Prognostic. Not predictive.

With selected variables

| | | | | | | |
|---|---|---|---|---|---|---|
| Treatment | −0.25539 | 0.161 | 0.775 | 0.565 | 1.063 | 0.113 |
| PMK2MN | −0.12011 | 0.063 | 0.887 | 0.783 | 1.004 | 0.058 |
| PMK2MN_Treatment | 0.12159 | 0.081 | 1.129 | 0.964 | 1.324 | 0.133 |
| Disease Stage | −0.04812 | 0.138 | 0.953 | 0.727 | 1.250 | 0.728 |
| T of TNM | 0.37258 | 0.150 | 1.451 | 1.081 | 1.949 | 0.013 |
| Performance Status | 0.16771 | 0.099 | 1.183 | 0.973 | 1.437 | 0.092 |
| Sex | −0.26832 | 0.215 | 0.765 | 0.502 | 1.164 | 0.211 |
| N of TNM | 0.46844 | 0.117 | 1.598 | 1.270 | 2.010 | 0.000 |

Not Prognostic. Not predictive.

With adjustment variables

| | | | | | | |
|---|---|---|---|---|---|---|
| Treatment | −0.27680 | 0.162 | 0.758 | 0.552 | 1.041 | 0.087 |
| PMK2MN | −0.12883 | 0.064 | 0.879 | 0.776 | 0.996 | 0.044 |
| PMK2MN_Treatment | 0.11845 | 0.081 | 1.126 | 0.960 | 1.320 | 0.145 |

Prognostic. Not predictive.

MEAN Q SCORE SUM OF PMK2 CYTOPLASM & NUCLEAR
Adenocarcinoma
Without variables

| | | | | | | |
|---|---|---|---|---|---|---|
| Treatment | 0.16053 | 0.222 | 1.174 | 0.760 | 1.814 | 0.470 |
| PMK2MCNs | 0.02293 | 0.071 | 1.023 | 0.891 | 1.175 | 0.745 |
| PMK2MCNs_Treatment | −0.08879 | 0.089 | 0.915 | 0.768 | 1.090 | 0.321 |

Not Prognostic. Not predictive.

With selected variables

| | | | | | | |
|---|---|---|---|---|---|---|
| Treatment | −0.18738 | 0.231 | 0.829 | 0.527 | 1.303 | 0.417 |
| PMK2MCNs | −0.07522 | 0.063 | 0.928 | 0.819 | 1.050 | 0.236 |
| PMK2MCNs_Treatment | 0.05472 | 0.091 | 1.056 | 0.884 | 1.263 | 0.548 |
| Disease Stage | 0.01894 | 0.211 | 1.019 | 0.674 | 1.541 | 0.928 |
| T of TNM | 0.33054 | 0.169 | 1.392 | 0.999 | 1.938 | 0.050 |
| Performance Status | 0.27439 | 0.140 | 1.316 | 0.999 | 1.732 | 0.051 |
| Sex | −0.45346 | 0.180 | 0.635 | 0.446 | 0.905 | 0.012 |
| Age | 0.30193 | 0.109 | 1.352 | 1.092 | 1.675 | 0.006 |
| N of TNM | 0.86808 | 0.202 | 2.382 | 1.604 | 3.539 | 0.000 |
| Surgery | −0.43252 | 0.199 | 0.649 | 0.440 | 0.958 | 0.029 |

Not Prognostic. Not predictive.

With adjustment variables

| | | | | | | |
|---|---|---|---|---|---|---|
| Treatment | −0.19373 | 0.231 | 0.824 | 0.524 | 1.296 | 0.402 |
| PMK2MCNs | −0.08450 | 0.063 | 0.919 | 0.812 | 1.040 | 0.181 |
| PMK2MCNs_Treatment | 0.06236 | 0.092 | 1.064 | 0.889 | 1.274 | 0.497 |

Not Prognostic. Not predictive.

Squamous Cells
Without variables

| | | | | | | |
|---|---|---|---|---|---|---|
| Treatment | −0.21304 | 0.153 | 0.808 | 0.599 | 1.090 | 0.163 |
| PMK2MCNs | −0.05859 | 0.043 | 0.943 | 0.867 | 1.026 | 0.175 |
| PMK2MCNs_Treatment | 0.06052 | 0.052 | 1.062 | 0.959 | 1.177 | 0.246 |

Not Prognostic. Not predictive.

With selected variables

| | | | | | | |
|---|---|---|---|---|---|---|
| Treatment | −0.23493 | 0.154 | 0.791 | 0.585 | 1.069 | 0.127 |
| PMK2MCNs | −0.07815 | 0.042 | 0.925 | 0.851 | 1.005 | 0.065 |
| PMK2MCNs_Treatment | 0.07642 | 0.052 | 1.079 | 0.975 | 1.196 | 0.143 |
| Disease Stage | −0.04638 | 0.138 | 0.955 | 0.728 | 1.252 | 0.738 |
| T of TNM | 0.37181 | 0.150 | 1.450 | 1.080 | 1.948 | 0.013 |
| Performance Status | 0.16698 | 0.099 | 1.182 | 0.973 | 1.436 | 0.093 |
| Sex | −0.26736 | 0.214 | 0.765 | 0.503 | 1.165 | 0.213 |
| N of TNM | 0.46975 | 0.117 | 1.600 | 1.271 | 2.013 | 0.000 |

Not Prognostic. Not predictive.

With adjustment variables

| | | | | | | |
|---|---|---|---|---|---|---|
| Treatment | −0.25901 | 0.154 | 0.772 | 0.571 | 1.044 | 0.093 |
| PMK2MCNs | −0.08462 | 0.042 | 0.919 | 0.846 | 0.998 | 0.046 |
| PMK2MCNs_Treatment | 0.07540 | 0.052 | 1.078 | 0.974 | 1.194 | 0.147 |

Prognostic. Not predictive.

Overall Survival
MEAN Q SCORE PMK2 CYTOPLASM
Adenocarcinoma
Without variables

| | | | | | | |
|---|---|---|---|---|---|---|
| Treatment | 0.23402 | 0.201 | 1.264 | 0.853 | 1.872 | 0.243 |
| PMK2MC | 0.17100 | 0.236 | 1.186 | 0.747 | 1.885 | 0.469 |
| PMK2MC_Treatment | −0.44818 | 0.324 | 0.639 | 0.339 | 1.205 | 0.167 |

Not Prognostic. Not predictive.

With selected variables

| | | | | | | |
|---|---|---|---|---|---|---|
| Treatment | −0.11562 | 0.216 | 0.891 | 0.583 | 1.361 | 0.593 |
| PMK2MC | −0.24435 | 0.230 | 0.783 | 0.499 | 1.228 | 0.287 |
| PMK2MC_Treatment | −0.02811 | 0.375 | 0.972 | 0.466 | 2.027 | 0.940 |
| Disease Stage | 0.04721 | 0.214 | 1.048 | 0.689 | 1.596 | 0.826 |
| T of TNM | 0.30423 | 0.175 | 1.356 | 0.962 | 1.911 | 0.082 |
| Performance Status | 0.17503 | 0.140 | 1.191 | 0.905 | 1.568 | 0.212 |
| Sex | −0.38096 | 0.189 | 0.683 | 0.471 | 0.990 | 0.044 |
| Age | 0.36019 | 0.112 | 1.434 | 1.151 | 1.785 | 0.001 |
| N of TNM | 0.72340 | 0.204 | 2.061 | 1.383 | 3.072 | 0.000 |
| Surgery | −0.40813 | 0.201 | 0.665 | 0.448 | 0.986 | 0.042 |

Not Prognostic. Not predictive.

With adjustment variables

| | | | | | | |
|---|---|---|---|---|---|---|
| Treatment | −0.12497 | 0.217 | 0.883 | 0.577 | 1.349 | 0.564 |
| PMK2MC | −0.27110 | 0.230 | 0.763 | 0.486 | 1.198 | 0.239 |
| PMK2MC_Treatrnent | 0.00078 | 0.375 | 1.001 | 0.480 | 2.087 | 0.998 |

Not Prognostic. Not predictive.

Squamous Cells
Without variables

| | | | | | | |
|---|---|---|---|---|---|---|
| Treatment | −0.18020 | 0.141 | 0.835 | 0.633 | 1.101 | 0.202 |
| PMK2MC | −0.13683 | 0.123 | 0.872 | 0.686 | 1.109 | 0.265 |
| PMK2MC_Treatment | 0.13745 | 0.141 | 1.147 | 0.870 | 1.513 | 0.330 |

Not Prognostic. Not predictive.

With selected variables

| | | | | | | |
|---|---|---|---|---|---|---|
| Treatment | −0.21913 | 0.143 | 0.803 | 0.607 | 1.063 | 0.126 |
| PMK2MC | −0.22618 | 0.123 | 0.798 | 0.627 | 1.015 | 0.066 |
| PMK2MC_Treatment | 0.21511 | 0.143 | 1.240 | 0.936 | 1.642 | 0.133 |
| Disease Stage | 0.01457 | 0.139 | 1.015 | 0.773 | 1.332 | 0.916 |
| T of TNM | 0.35593 | 0.151 | 1.428 | 1.063 | 1.917 | 0.018 |
| Performance Status | 0.15844 | 0.100 | 1.172 | 0.962 | 1.427 | 0.115 |
| Sex | −0.31518 | 0.218 | 0.730 | 0.476 | 1.119 | 0.149 |
| N of TNM | 0.45392 | 0.117 | 1.574 | 1.253 | 1.978 | 0.000 |

Not Prognostic. Not predictive.

With adjustment variables

| | | | | | | |
|---|---|---|---|---|---|---|
| Treatment | −0.24015 | 0.143 | 0.787 | 0.594 | 1.041 | 0.093 |
| PMK2MC | −0.24385 | 0.121 | 0.784 | 0.618 | 0.994 | 0.045 |
| PMK2MC_Treatment | 0.21795 | 0.141 | 1.244 | 0.943 | 1.640 | 0.123 |

Not Prognostic. Not predictive.

MEAN Q SCORE OF PMK2 CYTOPLASM DIVIDED BY NUCLEAR
Adenocarcinoma
Without variables

| | | | | | | |
|---|---|---|---|---|---|---|
| Treatment | 0.16273 | 0.325 | 1.177 | 0.622 | 2.225 | 0.617 |
| PMK2MCNp | 63.68060 | 106.8 | 45E26 | 0.000 | 4E118 | 0.551 |
| PMK2MCNp_Treatment | −45.0797 | 151.2 | 0.000 | 0.000 | 1E109 | 0.766 |

Not Prognostic. Not predictive.

With selected variables

| | | | | | | |
|---|---|---|---|---|---|---|
| Treatment | −0.00839 | 0.351 | 0.992 | 0.498 | 1.975 | 0.981 |
| PMK2MCNp | −56.0753 | 114.1 | 0.000 | 0.000 | 61E71 | 0.623 |
| PMK2MCNp_Treatment | −54.6537 | 169.5 | 0.000 | 0.000 | 3E120 | 0.747 |
| Disease Stage | 0.01393 | 0.216 | 1.014 | 0.665 | 1.547 | 0.948 |
| T of TNM | 0.33161 | 0.178 | 1.393 | 0.983 | 1.974 | 0.062 |
| Performance Status | 0.18621 | 0.140 | 1.205 | 0.916 | 1.585 | 0.184 |
| Sex | −0.37648 | 0.188 | 0.686 | 0.474 | 0.993 | 0.046 |
| Age | 0.35451 | 0.111 | 1.425 | 1.147 | 1.772 | 0.001 |
| N of TNM | 0.73652 | 0.206 | 2.089 | 1.395 | 3.126 | 0.000 |
| Surgery | −0.42922 | 0.200 | 0.651 | 0.440 | 0.964 | 0.032 |

Not Prognostic. Not predictive.

With adjustment variables

| | | | | | | |
|---|---|---|---|---|---|---|
| Treatment | −0.04730 | 0.355 | 0.954 | 0.476 | 1.913 | 0.894 |
| PMK2MCNp | −62.7248 | 114.6 | 0.000 | 0.000 | 22E69 | 0.584 |
| PMK2MCNp_Treatment | −30.4744 | 171.1 | 0.000 | 0.000 | 3E132 | 0.859 |

Not Prognostic. Not predictive.

Squamous Cells
Without variables

| | | | | | | |
|---|---|---|---|---|---|---|
| Treatment | −0.32072 | 0.241 | 0.726 | 0.452 | 1.164 | 0.183 |
| PMK2MCNp | −46.6804 | 65.50 | 0.000 | 0.000 | 3E35 | 0.476 |
| PMK2MCNp_Treatment | 79.31045 | 79.97 | 28E33 | 0.000 | 3E102 | 0.321 |

Not Prognostic. Not predictive.

With selected variables

| | | | | | | |
|---|---|---|---|---|---|---|
| Treatment | −0.43829 | 0.244 | 0.645 | 0.400 | 1.040 | 0.072 |
| PMK2MCNp | −87.8784 | 65.48 | 0.000 | 0.000 | 38E16 | 0.180 |
| PMK2MCNp_Treatment | 126.2227 | 81.23 | 66E53 | 0.000 | 9E123 | 0.120 |
| Disease Stage | 0.00320 | 0.139 | 1.003 | 0.765 | 1.316 | 0.982 |
| T of TNM | 0.35093 | 0.150 | 1.420 | 1.058 | 1.907 | 0.019 |
| Performance Status | 0.16148 | 0.100 | 1.175 | 0.967 | 1.429 | 0.105 |
| Sex | −0.30254 | 0.218 | 0.739 | 0.482 | 1.133 | 0.166 |
| N of TNM | 0.45566 | 0.117 | 1.577 | 1.255 | 1.982 | 0.000 |

Not Prognostic. Not predictive.

With adjustment variables

| | | | | | | |
|---|---|---|---|---|---|---|
| Treatment | −0.49178 | 0.242 | 0.612 | 0.380 | 0.984 | 0.043 |
| PMK2MCNp | −91.2009 | 64.22 | 0.000 | 0.000 | 11E14 | 0.156 |
| PMK2MCNp_Treatment | 138.6074 | 80.48 | 16E59 | 0.000 | 5E128 | 0.085 |

Not Prognostic. Not predictive.

MEAN Q SCORE PMK2 NUCLEAR
Adenocarcinoma
Without variables

| | | | | | | |
|---|---|---|---|---|---|---|
| Treatment | 0.13652 | 0.243 | 1.146 | 0.712 | 1.845 | 0.574 |
| PMK2MN | −0.04799 | 0.109 | 0.953 | 0.770 | 1.179 | 0.658 |
| PMK2MN_Treatment | −0.04482 | 0.132 | 0.956 | 0.738 | 1.238 | 0.734 |

Not Prognostic. Not predictive.

With selected variables

| | | | | | | |
|---|---|---|---|---|---|---|
| Treatment | −0.22029 | 0.252 | 0.802 | 0.489 | 1.316 | 0.383 |
| PMK2MN | −0.13846 | 0.099 | 0.871 | 0.717 | 1.057 | 0.161 |
| PMK2MN_Treatment | 0.08028 | 0.134 | 1.084 | 0.833 | 1.410 | 0.550 |
| Disease Stage | 0.06036 | 0.216 | 1.062 | 0.696 | 1.622 | 0.780 |
| T of TNM | 0.28273 | 0.176 | 1.327 | 0.940 | 1.872 | 0.107 |
| Performance Status | 0.19209 | 0.140 | 1.212 | 0.921 | 1.595 | 0.171 |
| Sex | −0.37635 | 0.189 | 0.686 | 0.474 | 0.994 | 0.046 |
| Age | 0.35226 | 0.113 | 1.422 | 1.141 | 1.773 | 0.002 |
| N of TNM | 0.70877 | 0.204 | 2.031 | 1.363 | 3.028 | 0.000 |
| Surgery | −0.40194 | 0.204 | 0.669 | 0.449 | 0.998 | 0.049 |

Not Prognostic. Not predictive.

With adjustment variables

| | | | | | | |
|---|---|---|---|---|---|---|
| Treatment | −0.20972 | 0.251 | 0.811 | 0.496 | 1.326 | 0.403 |
| PMK2MN | −0.14974 | 0.099 | 0.861 | 0.710 | 1.044 | 0.129 |
| PMK2MN_Treatment | 0.07402 | 0.135 | 1.077 | 0.827 | 1.402 | 0.582 |

Not Prognostic. Not predictive.

Squamous Cells
Without variables

| | | | | | | |
|---|---|---|---|---|---|---|
| Treatment | −0.24844 | 0.162 | 0.780 | 0.568 | 1.072 | 0.125 |
| PMK2MN | −0.09502 | 0.065 | 0.909 | 0.801 | 1.033 | 0.143 |
| PMK2MN_Treatment | 0.10515 | 0.082 | 1.111 | 0.946 | 1.304 | 0.199 |

Not Prognostic. Not predictive.

With selected variables

| | | | | | | |
|---|---|---|---|---|---|---|
| Treatment | −0.28745 | 0.164 | 0.750 | 0.544 | 1.034 | 0.079 |
| PMK2MN | −0.13253 | 0.064 | 0.876 | 0.773 | 0.993 | 0.038 |
| PMK2MN_Treatment | 0.13528 | 0.082 | 1.145 | 0.975 | 1.344 | 0.098 |
| Disease Stage | 0.01108 | 0.139 | 1.011 | 0.771 | 1.327 | 0.936 |
| T of TNM | 0.35772 | 0.151 | 1.430 | 1.065 | 1.921 | 0.018 |
| Performance Status | 0.15896 | 0.101 | 1.172 | 0.962 | 1.428 | 0.114 |
| Sex | −0.32080 | 0.219 | 0.726 | 0.473 | 1.114 | 0.142 |
| N of TNM | 0.45180 | 0.116 | 1.571 | 1.251 | 1.973 | 0.000 |

Prognostic. Not predictive.

With adjustment variables

| | | | | | | |
|---|---|---|---|---|---|---|
| Treatment | −0.30558 | 0.164 | 0.737 | 0.534 | 1.016 | 0.063 |
| PMK2MN | −0.14216 | 0.064 | 0.867 | 0.765 | 0.984 | 0.027 |
| PMK2MN_Treatment | 0.13534 | 0.082 | 1.145 | 0.975 | 1.344 | 0.098 |

Prognostic. Not predictive.

MEAN Q SCORE SUM OF PMK2 CYTOPLASM & NUCLEAR
Adenocarcinoma
Without variables

| | | | | | | |
|---|---|---|---|---|---|---|
| Treatment | 0.18459 | 0.235 | 1.203 | 0.759 | 1.905 | 0.432 |
| PMK2MCNs | −0.00911 | 0.079 | 0.991 | 0.849 | 1.157 | 0.908 |
| PMK2MCNs_Treatment | −0.06329 | 0.098 | 0.939 | 0.775 | 1.136 | 0.516 |

Not Prognostic. Not predictive.

With selected variables

| | | | | | | |
|---|---|---|---|---|---|---|
| Treatment | −0.18926 | 0.245 | 0.828 | 0.512 | 1.337 | 0.439 |
| PMK2MCNs | −0.09485 | 0.071 | 0.910 | 0.791 | 1.046 | 0.183 |
| PMK2MCNs_Treatment | 0.04280 | 0.101 | 1.044 | 0.857 | 1.271 | 0.671 |
| Disease Stage | 0.06053 | 0.215 | 1.062 | 0.696 | 1.621 | 0.779 |
| T of TNM | 0.28626 | 0.175 | 1.331 | 0.944 | 1.877 | 0.102 |
| Performance Status | 0.18650 | 0.140 | 1.205 | 0.916 | 1.586 | 0.183 |
| Sex | −0.37802 | 0.189 | 0.685 | 0.473 | 0.993 | 0.046 |
| Age | 0.35435 | 0.112 | 1.425 | 1.144 | 1.776 | 0.002 |
| N of TNM | 0.71218 | 0.203 | 2.038 | 1.368 | 3.037 | 0.000 |
| Surgery | −0.40112 | 0.203 | 0.670 | 0.450 | 0.997 | 0.048 |

Not Prognostic. Not predictive.

With adjustment variables

| | | | | | | |
|---|---|---|---|---|---|---|
| Treatment | −0.18386 | 0.244 | 0.832 | 0.516 | 1.342 | 0.451 |
| PMK2MCNs | −0.10295 | 0.071 | 0.902 | 0.785 | 1.037 | 0.147 |
| PMK2MCNs_Treatment | 0.04016 | 0.101 | 1.041 | 0.854 | 1.269 | 0.691 |

Not Prognostic. Not predictive.

Squamous Cells
Without variables

| | | | | | | |
|---|---|---|---|---|---|---|
| Treatment | −0.22594 | 0.155 | 0.798 | 0.589 | 1.080 | 0.144 |
| PMK2MCNs | −0.05988 | 0.044 | 0.942 | 0.865 | 1.026 | 0.170 |
| PMK2MCNs_Treatment | 0.06355 | 0.053 | 1.066 | 0.961 | 1.182 | 0.228 |

Not Prognostic. Not predictive.

With selected variables

| | | | | | | |
|---|---|---|---|---|---|---|
| Treatment | −0.26677 | 0.156 | 0.766 | 0.564 | 1.041 | 0.088 |
| PMK2MCNs | −0.08727 | 0.043 | 0.916 | 0.843 | 0.997 | 0.042 |
| PMK2MCNs_Treatment | 0.08621 | 0.053 | 1.090 | 0.983 | 1.209 | 0.103 |
| Disease Stage | 0.01272 | 0.139 | 1.013 | 0.772 | 1.329 | 0.927 |
| T of TNM | 0.35688 | 0.151 | 1.429 | 1.064 | 1.919 | 0.018 |
| Performance Status | 0.15862 | 0.101 | 1.172 | 0.962 | 1.427 | 0.115 |
| Sex | −0.32036 | 0.218 | 0.726 | 0.473 | 1.114 | 0.143 |
| N of TNM | 0.45330 | 0.116 | 1.573 | 1.253 | 1.977 | 0.000 |

Prognostic. Not predictive.

With adjustment variables

| | | | | | | |
|---|---|---|---|---|---|---|
| Treatment | −0.28609 | 0.156 | 0.751 | 0.553 | 1.021 | 0.067 |
| PMK2MCNs | −0.09386 | 0.043 | 0.910 | 0.837 | 0.990 | 0.029 |
| PMK2MCNs_Treatment | 0.08669 | 0.053 | 1.091 | 0.984 | 1.209 | 0.099 |

Prognostic. Not predictive.

TABLE 1
DNA Repair and DNA Damage Response Markers

| | | DNA |
|---|---|---|
| | | repair and DNA |
| Name | DNARMARKER No. | Damage Pathway |
| hOGG1 | 1. | BER |
| hNTH1 | 2. | BER |
| hNEIL1 | 3. | BER |
| hNEIL2 | 4. | BER |
| hNEIL3 | 5. | BER |
| AAG | 6. | BER |
| UNG1 | 7. | BER |
| TDG | 8. | BER |
| MUTY | 9. | BER |
| MTH1 | 10. | BER |
| MBD4 | 11. | BER |
| APE1 | 12. | BER |
| XPG | 13. | BER |
| DNAPOLβ | 14. | BER |
| XRCC1 | 15. | BER |
| PARP1 | 16. | BER |
| DNAPOLδ1 | 17. | BER |
| DNAPOLδ2 | 18. | BER |
| DNAPOLδ3 | 19. | BER |
| DNAPOLδ4 | 20. | BER |
| DNAPOLδ5 | 21. | BER |
| DNAPOLε1 | 22. | BER |
| DNAPOLε2 | 23. | BER |
| DNAPOLε3 | 24. | BER |
| DNAPOLε4 | 25. | BER |
| DNAPOLε5 | 26. | BER |
| DNALigaseI | 27. | BER |
| PCNA | 28. | BER |
| UBC13 | 29. | BER |
| MMS2 | 30. | BER |
| FEN1 | 31. | BER |
| RFC1 | 32. | BER |
| RFC2 | 33. | BER |
| RFC3 | 34. | BER |
| RFC4 | 35. | BER |
| RFC5 | 36. | BER |
| DNALigase1 | 37. | BER |
| DNAligase3 | 38. | BER |
| Aprataxin (Aptx) | 39. | BER |
| XRCC1 | 40. | HR |
| PARP1 | 41. | HR |
| FEN1 | 42. | HR |
| DNA ligase1 | 43. | HR |
| SNM1 | 44. | HR |
| H2A | 45. | HR |
| RPA1 | 46. | HR |
| RPA2 | 47. | HR |
| RPA3 | 48. | HR |
| RAD51 | 49. | HR |
| XRCC2 | 50. | HR |
| XRCC3 | 51. | HR |
| RAD51L1 | 52. | HR |
| RAD51L2 | 53. | HR |
| RAD51L3 | 54. | HR |
| DMC1 | 55. | HR |
| RAD52 | 56. | HR |
| RAD54 | 57. | HR |
| MUS81 | 58. | HR |
| MMS4 | 59. | HR |
| EMSY | 60. | HR |
| BRCA1 | 61. | HR |
| BARD1 | 62. | HR |
| BLM | 63. | HR |
| BLAP75 | 64. | HR |
| SRS2 | 65. | HR |
| SAE2 | 66. | HR |
| ERCC1 | 67. | HR |
| TRF2 | 68. | HR/FA |
| BRCA2/FANCD1 | 69. | HR/FA |
| FANCA | 70. | HR/FA |
| FANCB | 71. | HR/FA |
| FANCC | 72. | HR/FA |
| FANCD1 | 73. | HR/FA |
| FANCD2 | 74. | HR/FA |
| FANCE | 75. | HR/FA |
| FANCF | 76. | HR/FA |
| FANCG | 77. | HR/FA |
| FANCJ | 78. | HR/FA |
| FANCL | 79. | HR/FA |
| FANCM | 80. | HR/FA |
| hHef1 | 81. | HR/FA |
| FANCI | 82. | HR/FA |
| USP1 | 83. | HR/FA |
| PALB2/FANCN | 84. | HR/FA |
| DNMT1 | 85. | MMR |
| hMLH1 | 86. | MMR |
| hPMS2 | 87. | MMR |
| hPMS1 | 88. | MMR |
| GTBP (hMSH6) | 89. | MMR |
| hMSH2 | 90. | MMR |
| hMSH3 | 91. | MMR |
| HMGB1 | 92. | MMR |
| MSH4 | 93. | MMR |
| MSH5 | 94. | MMR |
| EXO1 | 95. | MMR |
| DNAPOLδ1 | 96. | MMR |
| DNAPOLδ2 | 97. | MMR |
| DNAPOLδ3 | 98. | MMR |
| DNAPOLδ4 | 99. | MMR |
| DNAPOLδ5 | 100. | MMR |
| DNAPOLε1 | 101. | MMR |
| DNAPOLε2 | 102. | MMR |
| DNAPOLε3 | 103. | MMR |
| DNAPOLε4 | 104. | MMR |
| DNAPOLε5 | 105. | MMR |
| DNA Ligase I | 106. | MMR |
| PCNA | 107. | MMR |
| RPA1 | 108. | MMR |
| RPA2 | 109. | MMR |
| RPA3 | 110. | MMR |
| MUTY | 111. | MMR |
| MRE11 | 112. | DDR |
| RAD50 | 113. | DDR |
| NBS1 | 114. | DDR |
| H2A | 115. | DDR |
| ATM | 116. | DDR |
| P53 | 117. | DDR |
| SMC1 | 118. | DDR |
| ATF2 | 119. | DDR |
| CHK1 | 120. | DDR |
| CHK2 | 121. | DDR |
| MAPKAP Kinase2 | 122. | DDR |
| RPA1 | 123. | DDR |
| RPA2 | 124. | DDR |
| RPA3 | 125. | DDR |
| RAD17 | 126. | DDR |
| RFC1 | 127. | DDR |
| RFC2 | 128. | DDR |
| RFC3 | 129. | DDR |
| RFC4 | 130. | DDR |
| RFC5 | 131. | DDR |
| RAD9 | 132. | DDR |
| RAD1 | 133. | DDR |
| HUS1 | 134. | DDR |
| ATRIP | 135. | DDR |
| ATR | 136. | DDR |
| MDC1 | 137. | DDR |
| CLASPIN | 138. | DDR |
| TOPB1 | 139. | DDR |
| BRCC36 | 140. | DDR |
| BLM | 141. | DDR |
| SRS2 | 142. | DDR |
| SAE2 | 143. | DDR |
| P53BP1 | 144. | DDR |
| ING1 | 145. | DDR |
| ING2 | 146. | DDR |
| SMC1 | 147. | DDR |
| BLAP75 | 148. | DDR |
| BACH1 | 149. | DDR |
| BRCA1 | 150. | DDR |
| BRCA2 | 151. | DDR |
| BARD1 | 152. | DDR |
| RAP80 | 153. | DDR |
| Abraxas | 154. | DDR |
| CDT1 | 155. | DDR |
| RPB8 | 156. | DDR |
| PPM1D | 157. | DDR |
| GADD45 | 158. | DDR |
| DTL/CDT2 | 159. | DDR |
| HCLK2 | 160. | DDR |
| CTIP | 161. | DDR |
| BAAT1 | 162. | DDR |
| HDM2/MDM2 | 163. | DDR |
| APLF (aprataxin- and PNK- | 164. | DDR |
| like factor) | | |
| 14-3-3 σ | 165. | DDR |
| Cdc25A | 166. | DDR |
| Cdc25B | 167. | DDR |
| Cdc25C | 168. | DDR |
| PBIP1 | 169. | DDR |
| H2A | 170. | NER |
| XPC | 171. | NER |
| HR23A | 172. | NER |
| HR23B | 173. | NER |
| DDB1 | 174. | NER |
| DDB2 | 175. | NER |
| XPD | 176. | NER |
| XPB | 177. | NER |
| XPG | 178. | NER |
| CSA | 179. | NER |
| CSB | 180. | NER |
| XPA | 181. | NER |
| XPF | 182. | NER |
| ERCC1 | 183. | NER |
| RNAPolymerase2 | 184. | NER |
| GTF2H1 | 185. | NER |
| GTF2H2 | 186. | NER |
| GTF2H3 | 187. | NER |
| GTF2H4 | 188. | NER |
| GTF2H5 | 189. | NER |
| MNAT1 | 190. | NER |
| MAT1 | 191. | NER |
| CDK7 | 192. | NER |
| CyclinH | 193. | NER |
| PCNA | 194. | NER |
| RFC1 | 195. | NER |
| RFC2 | 196. | NER |
| RFC3 | 197. | NER |
| RFC4 | 198. | NER |
| RFC5 | 199. | NER |
| DNAPOLδ1 | 200. | NER |
| DNAPOLδ2 | 201. | NER |
| DNAPOLδ3 | 202. | NER |
| DNAPOLδ4 | 203. | NER |
| DNAPOLδ5 | 204. | NER |
| DNAPOLε1 | 205. | NER |
| DNAPOLε2 | 206. | NER |
| DNAPOLε3 | 207. | NER |
| DNAPOLε4 | 208. | NER |
| DNAPOLε5 | 209. | NER |
| DNALigaseI | 210. | NER |
| DNAPOLη | 211. | TLS |
| DNAPOLι | 212. | TLS |
| DNAPOLκ | 213. | TLS |
| REV1 | 214. | TLS |
| DNAPOLζ | 215. | TLS |
| DNAPOLθ | 216. | TLS |
| PCNA | 217. | TLS |
| UBC13 | 218. | TLS |
| MMS2 | 219. | TLS |
| RAD5 | 220. | TLS |
| hRAD6A | 221. | TLS |
| hRAD6B | 222. | TLS |
| RAD18 | 223. | TLS |
| WRN | 224. | TLS |
| USP1 | 225. | TLS |
| SIRT6 | 226. | NHEJ |
| H2A | 227. | NHEJ |
| ARP4 | 228. | NHEJ |
| ARP8 | 229. | NHEJ |
| Ino80 | 230. | NHEJ |
| SWR1 | 231. | NHEJ |
| KU70 | 232. | NHEJ |
| KU80 | 233. | NHEJ |
| DNAPKcs | 234. | NHEJ |
| Artemis | 235. | NHEJ |
| PSO2 | 236. | NHEJ |
| XRCC4 | 237. | NHEJ |
| DNA LIGASE4 | 238. | NHEJ |
| XLF | 239. | NHEJ |
| DNAPOLλ | 240. | NHEJ |
| PNK | 241. | NHEJ |
| METNASE | 242. | NHEJ |
| TRF2 | 243. | NHEJ |
| MGMT | 244. | Non-classified |
| TDP1 | 245. | Non-classified |
| DNAPOLμ | 246. | Non-classified |
| hABH1 | 247. | Non-classified |
| hABH2 | 248. | Non-classified |
| hABH3 | 249. | Non-classified |
| hABH4 | 250. | Non-classified |
| hABH5 | 251. | Non-classified |
| hABH6 | 252. | Non-classified |
| hABH7 | 253. | Non-classified |
| hABH8 | 254. | Non-classified |
| TOPO1 | 255. | Non-classified |
| TOPOII | 256. | Non-classified |
| UBC9 | 257. | Non-classified |
| UBL1 | 258. | Non-classified |
| MMS21 | 259. | Non-classified |

TABLE 2
Non-small Cell Lung Cancer DNA Repair and DNA Damage Response
Markers

| | | DNA repair and DNA |
|---|---|---|
| Name | DNARMARKER No. | Damage Pathway |
| APE1 | 12 | BER |
| PARP1 | 16 | BER |
| PAR [poly ADP ribose | 260 | BER |
| modification] | | |
| RAD51 | 49 | HR |
| BRCA1 | 61 | HR |
| FANCD2 | 74 | HR/FA |
| hMLH1 | 86 | MMR |
| hMSH2 | 90 | MMR |
| H2AX [phosphorylation] | 115 | DDR |
| ATM | 116 | DDR |
| P53 | 117 | DDR |
| MAPKAP Kinase2 | 122 | DDR |
| [phosphorylation] | | |
| XPF | 182 | NER |
| ERCC1 | 183 | NER |
| POLε | 211 | TLS |
| HSP27 [phosphorylation] | 261 | DDR |
| Ki67 | 262 | CP |
| EGFRvIII | 263 | Onc |
| NQO1 | 264 | DT |
| c-met | 265 | Onc |

TABLE 3
Non-small Cell Lung Cancer Biomarkers for Use in Combined
Algorithms with DNA Repair and DNA Damage Response Markers

| | BioMARKER | |
|---|---|---|
| Name | No. | Function |
| RRM1 | 266 | DNA Base Synthesis |
| RRM2 | 267 | DNA Base Synthesis |
| CD133 | 268 | Membrane glycoprotein-cell adhesion |
| P21/waf1 | 269 | Cylin-dependent kinase inhibitor |
| BCRP | 270 | |
| CD55 | 271 | Immune regulation (complement) |
| CD59 | 272 | Immune regulation (complement) |
| HER2 | 273 | Oncogenic receptor tyrosine kinase |
| P21/K-ras | 274 | Signal transduction |
| FHIT | 275 | Purine metabolism |
| MRP-7 | 276 | |
| CD34MVD | 277 | Membrane glycoprotein-cell adhesion |
| EMA | 278 | Epithelial membrane antigen |
| TIMP-1 | 279 | Inhibition of cell adhesion proteinases |
| TIMP-2 | 280 | Inhibition of cell adhesion proteinases |
| BCL2 | 281 | Early role in apoptosis cascade |
| Caspase 9 | 282 | Apoptosis gatekeeper-cleavage of |
| | | PARP |
| Cytokeratin | 283 | Cytosketal architecture |
| Cyclooxygenase 2 | 284 | Prostaglandin biosynthesis |
| MMP-2 | 285 | Cell adhesion molecule proteinases - |
| | | metastasis |
| MMP-9 | 286 | Cell adhesion molecule proteinases - |
| | | metastasis |
| P16/ink4a | 287 | Cylin-dependent kinase inhibitor |
| P27/kip1 | 288 | Cylin-dependent kinase inhibitor |
| VEGF | 289 | Soluble growth factor |
| β-catenin | 290 | Signal Transducer |
| β-tubulin 2 | 291 | Microtubule structure and regulation |

TABLE 4
Non-small Cell Lung Cancer Biomarkers (Gene Amplification/Deletion
via FISH) for Use in Combined Algorithms with DNA Repair and DNA
Damage Response Markers

| Name | BioMARKER No. | |
|---|---|---|
| MDM2 | 163 | Tumor supperssor |
| EGFR | 263 | Growth Factor Receptor |
| | | Kinase |
| c-met | 265 | Tyrosine Kinase, Signaling |
| ALK | 292 | Tyrosine Kinase, Signaling |

### REFERENCES

Ceppi, P. et al. (2009) Polymerase H mRNA Expression Predicts Survival of Non-Small Cell Lung Cancer Patients Treated with Platinum-Based Chemotherapy. Clinical Cancer Research 15; 1039-1045.
Gazdar A. F. (2007) DNA Repair and Survival in Lung Cancer—The Two Faces of Janus. NEJM 356, 771-773.
Holm B, et al. (2009) Different Impact of Excision Repair Cross-Complementation Group 1 on Survival in Male and Female Patients With Inoperable Non-Small-Cell Lung Cancer Treated With Carboplatin and Gemcitabine. J Clin Oncol. [Epub ahead of print]
Ko, J.-C. et al. (2008) Role of repair protein Rad51 in regulating the response to gefitinib in human non-small cell lung cancer cells. Molecular Cancer Therapeutics 7, 3632-3641.
Kang et al., 2009 The prognostic significance of ERCC1, BRCA1, XRCC1, and betaIII-tubulin expression in patients with non-small cell lung cancer treated by platinum- and taxane-based neoadjuvant chemotherapy and surgical resection; Lung Cancer 2009 Aug. 14
Lee, K.-H., et al. (2008) ERCC1 expression by immunohistochemistry and EGFR mutations in resected non-small cell lung cancer. Lung Cancer 60, 401-407.
Olaussen, K. A. et al. (2006) DNA Repair by ERCC1 in Non-Small-Cell Lung Cancer and Cisplatin-Based Adjuvant Chemotherapy. NEJM 355, 983-991.
Scartozzi, M. et al. (2006) Mismatch repair system (MMR) status correlates with response and survival in non-small cell lung cancer (NSCLC) patients. Lung Cancer 53, 103-109.
Singh N and Aggarwal A N. (2009) ERCC1 and RRM1 Expression in nonsmall cell lung cancer—the good, the bad and the unknown. J Thorac Oncol. 4:1042-3.
Takenaka, T. et al. (2007) Combined evaluation of Rad51 and ERCC1 expressions for sensitivity to platinum agents in non-small cell lung cancer. Int. J. Cancer: 121, 895-900.
Taron, M. et al. (2004) BRCA1 mRNA expression levels as an indicator of chemoresistance in lung cancer. Human Molecular Genetics, 13, 2443-2449.
Wang, D. et al. (2009) APE1 overexpression is associated with cisplatin resistance in non-small cell lung cancer and targeted inhibition of APE1 enhances the activity of cisplatin in A549 cells. Lung Cancer, in press
Wang, et al., 2009; Med Oncol. 2009 Jun. 2. Positive expression of ERCC1 predicts a poorer platinum-based treatment outcome in Chinese patients with advanced non-small-cell lung cancer.
Zheng, Z. et al. (2007) DNA Synthesis and Repair Genes RRM1 and ERCC1 in Lung Cancer. NEJM 356, 800-808.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for predicting the response to chemotherapy and/or survivability of a subject having a non-small cell lung cancer comprising
a) measuring the level of an effective amount of one or more DNARMARKERS in a sample from the subject; and
b) comparing the level of the effective amount of the one or more DNARMARKERS to a reference value.

2. A method of accessing the effectiveness of a chemotherapeutic agent treatment of a subject having a non-small cell lung cancer comprising
a) measuring the level of an effective amount of one or more DNARMARKERS in a sample from the subject; and
b) comparing the level of the effective amount of the one or more DNARMARKERS to a reference value.

3. A method of monitoring the a chemotherapeutic agent treatment of a subject with non-small cell lung cancer comprising
a) detecting the level of an effective amount of one or more DNARMARKERS in a first sample from the subject at a first period of time;
b) detecting the level of an effective amount of one or more DNARMARKERS in a second sample from the subject at a second period of time; and
c) comparing the level of the effective amount of one or more DNARMARKERS detected in step (a) to the amount detected in step (b), or to a reference value.

4. The method of claim 1, wherein the survivability is disease free survival or overall survival.

5. The method of claim 1, wherein the chemotherapy is cisplatin.

6. The method of claim 1, wherein the lung cancer is squamous cell lung cancer.

7. An algorithm that is derived from any combination of biomarkers comprising the list of biomarkers in Table 1, Table 2, Table 3 or Table 4 which specifies how the biomarkers are associated in relation to the other biomarkers in the panel, such that the biomarker algorithm indicates a predictive or prognostic value in treatment response of head and neck cancer.

8. The method of claim 1, wherein said DNARMARKER is selected from Table 2.

9. The method of claim 1, further comprising detecting one or more additional marker from Table 3 or 4.

10. The method of claim 1, wherein said DNARMARKER is XPF, FANCD2, pMK2, PAR, p53, ERCC1, ATM, MLH1, PARP1, pH2AX, pHSP27, BRCA1, BRCA2, RAD51, NQO1, or MSH2.

11. The method of claim 1, wherein the DNARMARKER is MSH2, p53, pMK2(n), pMK2(c=n), or ATM.

12. The method of claim 1, wherein the DNARMARKER is MSH2, p53, or ATM.

13. The method of claim 1, wherein the DNARMARKER is p53, pMK2 ERCC1, PARP1 or ATM.

14. The method of claim 1, further comprising measuring at least one clinical parameter.

15. The method of claim 1, wherein said DNARMARKER is detected by quantifiable gene copy number variation.

16. The method of claim 1, wherein said DNARMARKER is detectable by fluorescence in situ hybridization and/or colorimetric in situ hybridization.

17. The method of claim 1, wherein said DNARMARKER is detectable by immunofluorescence and/or immunohistochemistry.

18. The method of claim 1, wherein said DNARMARKER is detectable by Protein Nucleic Acid (PNA) hybridization.

19. The method of claim 1, wherein said DNARMARKER is detectable by high-throughput next generation sequencing.

20. The method of claim 1, wherein said DNARMARKER algorithms are invented from combinations of any DNARMARKER listed herein.

21. The method of claim 1, wherein said chemotherapeutic agent is carboplatin, or one of the related class of platinum drugs, or taxane, or one of the class of taxanes, or both.

22. The method of claim 1, wherein said chemotherapeutic agent is chemoradiotherapy.
